(19) United States
(12) Patent Application Publication
(10) Pub. No.: US 2012/0095005 A1
(43) Pub. Date: Apr. 19, 2012

(54) Fused Bicyclic Pyrazole Derivatives As Kinase Inhibitors

(75) Inventors: Daniel Rees Allen, Essex (GB); Julien Alistair Brown, Berkshire (GB); Roland Burli, Essex (GB); Alan Findlay Haughan, Essex (GB); Barry John Langham, Berkshire (GB); Mizio Matteucci, Essex (GB); Andrew Pate Owens, Essex (GB); Gilles Raphy, Essex (GB); Andrew Sharpe, Essex (GB)

(73) Assignee: UCB PHARMA S.A., Brussels (BE)

(21) Appl. No.: 13/321,305

(22) PCT Filed: May 19, 2010
(86) PCT No.: PCT/GB10/01000

§ 371 Date: Jan. 4, 2012

(30) Foreign Application Priority Data
May 22, 2009 (GB) 0908957.4

(51) Int. Cl. A61K 31/53; A61K 31/519; A61P 35/00; A61P 29/00; A61P 37/06; A61P 9/00; A61P 25/28; A61P 3/00; A61P 27/02; A61P 11/06; A61P 1/00; A61P 25/00; A61P 17/06; A61P 7/02; A61P 9/12; A61P 9/04; A61P 25/16; A61P 3/04; A61P 3/10; A61P 35/02; C07D 487/04

(52) U.S. Cl. 514/245; 544/281; 544/212; 514/255.05; 514/252.16; 514/259.3; 514/252.11

(57) ABSTRACT

A series of substituted pyrazolo[1,5-α][1,3,5]triazine and pyrazolo[1,5-αj-pyrimidine derivatives, being selective inhibitors of PI3 kinase enzymes, are accordingly of benefit in medicine, for example in the treatment of inflammatory, autoimmune, cardiovascular, neurodegenerative, metabolic, oncological, nociceptive or ophthalmic conditions. Formula (I).

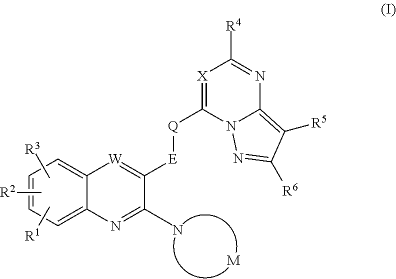

The present invention relates to a class of fused bicyclic pyrazole derivatives, and to their use in therapy. More particularly, the compounds in accordance with the present invention are substituted pyrazolo[1,5-a][1,3,5]triazine and pyrazolo[1,5-a]pyrimidine derivatives. These compounds are selective inhibitors of phosphoinositide 3-kinase (PI3K) enzymes, and are accordingly of benefit as pharmaceutical agents, especially in the treatment of adverse inflammatory, autoimmune, cardiovascular, neurodegenerative, metabolic, oncological, nociceptive and ophthalmic conditions.

The PI3K pathway is implicated in a variety of physiological and pathological functions that are believed to be operative in a range of human diseases. Thus, PI3Ks provide a critical signal for cell proliferation, cell survival, membrane trafficking, glucose transport, neurite outgrowth, membrane ruffling, superoxide production, actin reorganization and chemotaxis (cf. S. Ward et al., Chemistry & Biology, 2003, 10, 207-213; and S. G. Ward & P. Finan, Current Opinion in Pharmacology, 2003, 3, 426-434); and are known to be involved in the pathology of cancer, and metabolic, inflammatory and cardiovascular diseases (cf. M. P. Wymann et al., Trends in Pharmacol. Sci., 2003, 24, 366-376). Aberrant upregulation of the PI3K pathway is implicated in a wide variety of human cancers (cf. S. Brader & S. A. Eccles, Tumori, 2004, 90, 2-8).

The compounds in accordance with the present invention, being potent and selective PI3K inhibitors, are therefore beneficial in the treatment and/or prevention of various human ailments. These include autoimmune and inflammatory disorders such as rheumatoid arthritis, multiple sclerosis, asthma, inflammatory bowel disease, psoriasis and transplant rejection; cardiovascular disorders including thrombosis, cardiac hypertrophy, hypertension, and irregular contractility of the heart (e.g. during heart failure); neurodegenerative disorders such as Alzheimer's disease, Parkinson's disease, Huntington's disease, stroke, amyotrophic lateral sclerosis, spinal cord injury, head trauma and seizures; metabolic disorders such as obesity and type 2 diabetes; oncological conditions including leukaemia, glioblastoma, lymphoma, melanoma, and human cancers of the liver, bone, skin, brain, pancreas, lung, breast, stomach, colon, rectum, prostate, ovary and cervix; pain and nociceptive disorders; and ophthalmic disorders including age-related macular degeneration (ARMD).

In addition, the compounds in accordance with the present invention may be beneficial as pharmacological standards for use in the development of new biological tests and in the search for new pharmacological agents. Thus, the compounds of this invention may be useful as radioligands in assays for detecting compounds capable of binding to human PI3K enzymes.

WO 2008/118454, WO 2008/118455 and WO 2008/118468 describe various series of quinoline and quinoxaline derivatives that are structurally related to each other and are stated to be useful to inhibit the biological activity of human PI3Kδ and to be of use in treating PI3K-mediated conditions or disorders.

Copending international patent application PCT/GB2008/004171, published on 2 Jul. 2009 as WO 2009/081105, copending international patent application PCT/GB2009/002504, published on 29 Apr. 2010 as WO 2010/046639 (claiming priority from United Kingdom patent application 0819593.5), copending international patent application PCT/GB2010/000243 (claiming priority from United Kingdom patent applications 0902450.6 and 0914533.5) and copending international patent application PCT/GB2010/000361 (claiming priority from United Kingdom patent applications 0903949.6 and 0915586.2) describe separate classes of fused bicyclic heteroaryl derivatives as selective inhibitors of PI3K enzymes that are of benefit in the treatment of adverse inflammatory, autoimmune, cardiovascular, neurodegenerative, metabolic, oncological, nociceptive and ophthalmic conditions.

Copending international patent application PCT/US2009/005380, published on 1 Apr. 2010 as WO 2010/036380, describes a family of quinoline and quinoxaline derivatives that are stated to bind specifically to a PI3 kinase and to be of use in a method of treating a medical condition mediated by a type I PI3 kinase.

None of the prior art available to date, however, discloses or suggests the precise structural class of fused bicyclic pyrazole derivatives as provided by the present invention.

The compounds of the present invention are potent and selective PI3K inhibitors having a binding affinity (IC₅₀) for the human PI3Kα and/or PI3Kβ and/or PI3Kγ and/or PI3Kδ isoform of 50 μM or less, generally of 20 μM or less, usually of 5 μM or less, typically of 1 μM or less, suitably of 500 nM or less, ideally of 100 nM or less, and preferably of 20 nM or less (the skilled person will appreciate that a lower IC₅₀ figure denotes a more active compound). The compounds of the invention may possess at least a 10-fold selective affinity, typically at least a 20-fold selective affinity, suitably at least a 50-fold selective affinity, and ideally at least a 100-fold selective affinity, for the human PI3Kα and/or PI3Kβ and/or PI3Kγ and/or PI3Kδ isoform relative to other human kinases.

The present invention provides a compound of formula (I) or an N-oxide thereof, or a pharmaceutically acceptable salt or solvate thereof:

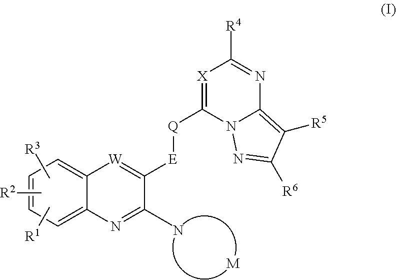

wherein

X represents N or C—R⁷;

E represents an optionally substituted straight or branched C₁₋₄ alkylene chain;

Q represents oxygen, sulfur, N—R⁸ or a covalent bond;

M represents the residue of an optionally substituted saturated five-, six- or seven-membered monocyclic ring containing one nitrogen atom and 0, 1, 2 or 3 additional heteroatoms independently selected from N, O and S, but containing no more than one O or S atom, which ring may be optionally fused to an optionally substituted heteroaromatic ring;

W represents C—R⁹ or N;

R¹, R² and R³ independently represent hydrogen, halogen, cyano, nitro, C₁₋₆ alkyl, trifluoromethyl, aryl(C₁₋₆)alkyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkyl-amino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl;

R⁴, R⁵, R⁶ and R⁷ independently represent C₁₋₆ alkyl, aryl, aryl(C₁₋₆)alkyl, heteroaryl or heteroaryl(C₁₋₆)alkyl, any of which groups may be optionally substituted by one or more substituents; or hydrogen, halogen, trifluoromethyl, —OR^{a}, —SR^{a}, —SOR^{a}, —SO₂R^{a}, —NR^{b}R^{c}, —NR^{c}COR^{d}, —NR^{c}CO₂R^{d}, —NR^{c}SO₂R^{e}, —COR^{d}, —CO₂R^{d}, —CONR^{b}R^{c} or —SO₂NR^{b}R^{c};

R⁸ represents hydrogen or C₁₋₆ alkyl;

R⁹ represents hydrogen, halogen, C₁₋₆ alkyl or C₁₋₆ alkoxy;

R^{a} represents C₁₋₆ alkyl, difluoromethyl or trifluoromethyl;

R^{b} represents hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl or heteroaryl(C₁₋₆)alkyl, any of which groups may be optionally substituted by one or more substituents;

R^{c} represents hydrogen, C₁₋₆ alkyl or C₃₋₇ cycloalkyl;

R^{d} represents hydrogen or C₁₋₆ alkyl; and

R^{e} represents C₁₋₆ alkyl.

The present invention also provides a compound of formula (I) as depicted above or an N-oxide thereof, or a pharmaceutically acceptable salt or solvate thereof, wherein

M represents the residue of an optionally substituted saturated five-, six- or seven-membered monocyclic ring containing one nitrogen atom and 0, 1, 2 or 3 additional heteroatoms independently selected from N, O and S, but containing no more than one O or S atom; and

X, E, Q, W, R¹, R², R³, R⁴, R⁵ and R⁶ are as defined above.

Where any of the groups in the compounds of formula (I) above is stated to be optionally substituted, this group may be unsubstituted, or substituted by one or more substituents. Typically, such groups will be unsubstituted, or substituted by one or two substituents.

For use in medicine, the salts of the compounds of formula (I) will be pharmaceutically acceptable salts. Other salts may, however, be useful in the preparation of the compounds of the invention or of their pharmaceutically acceptable salts. Suitable pharmaceutically acceptable salts of the compounds of this invention include acid addition salts which may, for example, be formed by mixing a solution of the compound of the invention with a solution of a pharmaceutically acceptable acid such as hydrochloric acid, sulphuric acid, methanesulphonic acid, fumaric acid, maleic acid, succinic acid, acetic acid, benzoic acid, citric acid, tartaric acid or phosphoric acid. Furthermore, where the compounds of the invention carry an acidic moiety, e.g. carboxy, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The present invention includes within its scope solvates of the compounds of formula (I) above. Such solvates may be formed with common organic solvents, e.g. hydrocarbon solvents such as benzene or toluene; chlorinated solvents such as chloroform or dichloromethane; alcoholic solvents such as methanol, ethanol or isopropanol; ethereal solvents such as diethyl ether or tetrahydrofuran; or ester solvents such as ethyl acetate. Alternatively, the solvates of the compounds of formula (I) may be formed with water, in which case they will be hydrates.

Suitable alkyl groups which may be present on the compounds of the invention include straight-chained and branched C₁₋₆ alkyl groups, for example C₁₋₄ alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulphonyl” and “C₁₋₆ alkylamino” are to be construed accordingly.

The expression “C₁₋₃ alkylene chain” refers to a divalent straight or branched alkylene chain containing 1 to 3 carbon atoms. Typical examples include methylene, ethylene, methylmethylene, ethylmethylene and dimethylmethylene.

Specific C₃₋₇ cycloalkyl groups are cyclopropyl, cyclobutyl, cyclopentyl, cyclohexyl and cycloheptyl.

Suitable aryl groups include phenyl and naphthyl, preferably phenyl.

Suitable aryl(C₁₋₆)alkyl groups include benzyl, phenylethyl, phenylpropyl and naphthylmethyl.

Suitable heterocycloalkyl groups, which may comprise benzo-fused analogues thereof, include azetidinyl, tetrahydrofuranyl, dihydrobenzofuranyl, pyrrolidinyl, indolinyl, oxazolidinyl, thiazolidinyl, imidazolidinyl, tetrahydropyranyl, chromanyl, piperidinyl, 1,2,3,4-tetrahydroquinolinyl, 1,2,3,4-tetrahydroisoquinolinyl, piperazinyl, 1,2,3,4-tetrahydroquinoxalinyl, homopiperazinyl, morpholinyl, benzoxazinyl and thiomorpholinyl.

Suitable heteroaryl groups include furyl, benzofuryl, dibenzofuryl, thienyl, benzothienyl, dibenzothienyl, pyrrolyl, indolyl, pyrrolo[2,3-b]pyridinyl, pyrrolo[3,2-c]pyridinyl, pyrazolyl, pyrazolo[1,5-a]pyridinyl, pyrazolo[3,4-d]pyrimidinyl, indazolyl, oxazolyl, benzoxazolyl, isoxazolyl, thiazolyl, benzothiazolyl, isothiazolyl, imidazolyl, benzimidazolyl, imidazo[1,2-a]pyridinyl, imidazo[4,5-b]pyridinyl, purinyl, imidazo[1,2-a]pyrimidinyl, imidazo[1,2-a]pyrazinyl, oxadiazolyl, thiadiazolyl, triazolyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halogen” as used herein is intended to include fluorine, chlorine, bromine and iodine atoms, typically fluorine, chlorine or bromine.

Where the compounds of formula (I) have one or more asymmetric centres, they may accordingly exist as enantiomers. Where the compounds of the invention possess two or more asymmetric centres, they may additionally exist as diastereomers. The invention is to be understood to extend to all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)⇄enol (CH═CHOH) tautomers or amide (NHC═O)⇄hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In one embodiment, X represents N. In another embodiment, X represents C—R⁷.

In one embodiment, W represents C—R⁹. In another embodiment, W represents N.

Specific sub-classes of compounds in accordance with the present invention are represented by the compounds of formula (IA), (IB), (IC) and (ID), preferably (IA) or (IC), especially (IA):

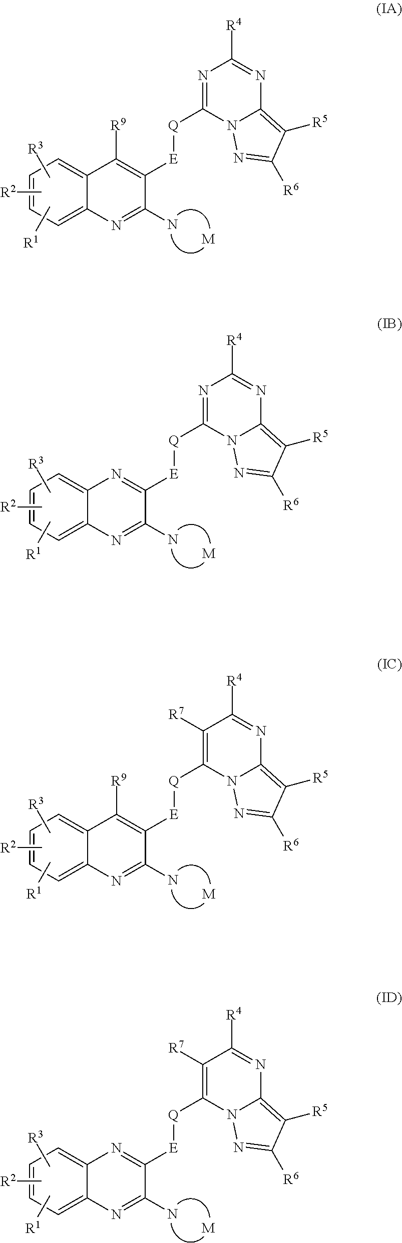

wherein E, Q, M, R¹, R², R³, R⁴, R⁵, R⁶, R⁷ and R⁹ are as defined above.

Typical values of E include methylene (—CH₂—), (methyl)methylene, ethylene (—CH₂CH₂—), (ethyl)methylene, (dimethyl)methylene, (methyl)ethylene, (propyl)methylene and (dimethyl)ethylene, any of which chains may be optionally substituted by one or more substituents. Suitably, such chains are unsubstituted, monosubstituted or disubstituted. Preferably, such chains are unsubstituted or monosubstituted. In one embodiment, such chains are unsubstituted. In another embodiment, such chains are monosubstituted.

Examples of suitable substituents on the alkylene chain represented by E include trifluoromethyl, C₃₋₇ heterocycloalkyl, aryl, oxo, hydroxy, C₁₋₆ alkoxy, C₂₋₆ alkoxy-carbonyl(C₁₋₆)alkoxy, aminocarbonyl(C₁₋₆)alkoxy, trifluoromethoxy, amino, C₁₋₆ alkylamino, di(C₁₋₆)alkylamino, aminocarbonyl, C₁₋₆ alkylaminocarbonyl and di(C₁₋₆)alkylaminocarbonyl.

Examples of particular substituents on the alkylene chain represented by E include trifluoromethyl, pyrrolidinyl, piperidinyl, piperazinyl, morpholinyl, phenyl, oxo, hydroxy, ethoxy, ethoxycarbonylmethoxy, aminocarbonylmethoxy, trifluoromethoxy, amino, methylamino, dimethylamino, aminocarbonyl, methylaminocarbonyl and dimethylamino-carbonyl.

Suitably, E represents methylene or (methyl)methylene.

A particular value of E is (methyl)methylene, i.e. —CH(CH₃)—, in the (R) or (S) stereochemical configuration, preferably (S).

Another value of E is methylene, i.e. —CH₂—.

In one embodiment, Q represents oxygen. In another embodiment, Q represents sulfur. In a further embodiment, Q represents N—R⁸. In a still further embodiment, Q represents a covalent bond.

Suitably, M represents the residue of an optionally substituted saturated five-, six- or seven-membered monocyclic ring containing one nitrogen atom and 0, 1, 2 or 3 additional heteroatoms independently selected from N, O and S, but containing no more than one O or S atom.

In one embodiment, M represents the residue of an optionally substituted saturated five-membered monocyclic ring. In another embodiment, M represents the residue of an optionally substituted saturated six-membered monocyclic ring. In a further embodiment, M represents the residue of an optionally substituted saturated seven-membered monocyclic ring.

In one embodiment, the monocyclic ring of which M is the residue contains one nitrogen atom and no additional heteroatoms (i.e. it is an optionally substituted pyrrolidin-1-yl, piperidin-1-yl or hexahydroazepin-1-yl ring). In another embodiment, the monocyclic ring of which M is the residue contains one nitrogen atom and one additional heteroatom selected from N, O and S. In a further embodiment, the monocyclic ring of which M is the residue contains one nitrogen atom and two additional heteroatoms selected from N, O and S, of which not more than one is O or S. In a still further embodiment, the monocyclic ring of which M is the residue contains one nitrogen atom and three additional heteroatoms selected from N, O and S, of which not more than one is O or S.

Illustrative values of the monocyclic ring of which M is the residue include pyrrolidin-1-yl, oxazolidin-3-yl, piperidin-1-yl, piperazin-1-yl and morpholin-4-yl, any of which rings may be optionally substituted by one or more substituents.

Typical values of the monocyclic ring of which M is the residue include pyrrolidin-1-yl, morpholin-4-yl and piperazin-1-yl, any of which rings may be optionally substituted by one or more substituents.

Definitive values of the monocyclic ring of which M is the residue include pyrrolidin-1-yl, oxazolidin-3-yl, piperidin-1-yl and piperazin-1-yl, any of which rings may be optionally substituted by one or more substituents.

A suitable value of the monocyclic ring of which M is the residue is optionally substituted piperazin-1-yl.

In one embodiment, the monocyclic ring of which M is the residue is unsubstituted. In another embodiment, the monocyclic ring of which M is the residue is substituted by one or more substituents. In one subset of that embodiment, the monocyclic ring of which M is the residue is monosubstituted. In another subset of that embodiment, the monocyclic ring of which M is the residue is disubstituted.

Illustrative examples of suitable substituents on the monocyclic ring of which M is the residue include halogen, C₁₋₆ alkyl, heteroaryl, C₁₋₆ alkoxy, difluoromethoxy, trifluoromethoxy, C₁₋₆ alkoxy(C₁₋₆)alkyl, C₁₋₆ alkylthio, C₁₋₆ alkylsulphonyl, hydroxy, hydroxy(C₁₋₆)alkyl, amino(C₁₋₆)alkyl, cyano, trifluoromethyl, oxo, C₂₋₆ alkylcarbonyl, hydroxy(C₁₋₆)alkyloarbonyl, di(C₁₋₆)alkylamino(C₁₋₆)alkylcarbonyl, (C₃₋₇)cycloalkyl-carbonyl, heteroarylcarbonyl, carboxy, carboxy(C₁₋₆)alkyl, C₂₋₆ alkoxycarbonyl, C₂₋₆ alkoxycarbonyl(C₁₋₆)alkyl, amino, C₁₋₆ alkylamino, di(C₁₋₆)alkylamino, phenylamino, pyridinylamino, C₂₋₆ alkylcarbonylamino, hydroxy(C₁₋₆)alkylcarbonylamino, (C₁₋₆)alkyl-carbonylamino(C₁₋₆)alkyl, (C₃₋₇)cycloalkylcarbonylamino, C₂₋₆ alkoxycarbonylamino, aminocarbonyl, (C₁₋₆)alkylaminocarbonyl and di(C₁₋₆)alkylaminocarbonyl(C₁₋₆)alkyl.

Typical examples of suitable substituents on the monocyclic ring of which M is the residue include halogen, C₁₋₆ alkyl, C₁₋₆ alkoxy, difluoromethoxy, trifluoromethoxy, C₁₋₆ alkoxy(C₁₋₆)alkyl, C₁₋₆ alkylthio, C₁₋₆ alkylsulphonyl, hydroxy, hydroxy(C₁₋₆)alkyl, amino(C₁₋₆)alkyl, cyano, trifluoromethyl, oxo, C₂₋₆ alkylcarbonyl, carboxy, C₂₋₆ alkoxycarbonyl, amino, C₁₋₆ alkylamino, di(C₁₋₆)alkylamino, phenylamino, pyridinylamino, C₂₋₆ alkylcarbonylamino, C₂₋₆ alkoxycarbonylamino and aminocarbonyl.

Definitive examples of suitable substituents on the monocyclic ring of which M is the residue include heteroaryl, hydroxy(C₁₋₆)alkyl, oxo, C₂₋₆ alkylcarbonyl, hydroxy(C₁₋₆)-alkylcarbonyl, di(C₁₋₆)alkylamino(C₁₋₆)alkylcarbonyl, (C₃₋₇)cycloalkylcarbonyl, heteroarylcarbonyl, carboxy, carboxy(C₁₋₆)alkyl, C₂₋₆ alkoxycarbonyl, C₂₋₆ alkoxy-carbonyl(C₁₋₆)alkyl, hydroxy(C₁₋₆)alkylcarbonylamino, (C₁₋₆)alkylcarbonylamino(C₁₋₆)-alkyl, (C₃₋₇)cycloalkylcarbonylamino, aminocarbonyl, (C₁₋₆)alkylaminocarbonyl and di-(C₁₋₆)alkylaminocarbonyl(C₁₋₆)alkyl.

Illustrative examples of specific substituents on the monocyclic ring of which M is the residue include fluoro, chloro, bromo, methyl, ethyl, isopropyl, thiazolyl, pyridinyl, pyrazinyl, methoxy, isopropoxy, difluoromethoxy, trifluoromethoxy, methoxymethyl, methylthio, ethylthio, methylsulphonyl, hydroxy, hydroxymethyl, hydroxyethyl, aminomethyl, cyano, trifluoromethyl, oxo, acetyl, propionyl, tert-butylcarbonyl, hydroxyacetyl, (dimethylamino)acetyl, cyclopropylcarbonyl, thienylcarbonyl, pyridinylcarbonyl, carboxy, carboxymethyl, methoxycarbonyl, ethoxycarbonyl, tert-butoxy carbonyl, ethoxycarbonylmethyl, amino, methylamino, ethylamino, dimethylamino, phenylamino, pyridinylamino, acetylamino, hydroxyacetylamino, acetylaminomethyl, cyclopropylcarbonylamino, tert-butoxycarbonylamino, aminocarbonyl, methylaminocarbonyl and dimethylaminocarbonylmethyl.

Typical examples of specific substituents on the monocyclic ring of which M is the residue include fluoro, chloro, bromo, methyl, ethyl, isopropyl, methoxy, isopropoxy, difluoromethoxy, trifluoromethoxy, methoxymethyl, methylthio, ethylthio, methylsulphonyl, hydroxy, hydroxymethyl, hydroxyethyl, aminomethyl, cyano, trifluoromethyl, oxo, acetyl, carboxy, methoxycarbonyl, ethoxycarbonyl, tert-butoxy carbonyl, amino, methylamino, ethylamino, dimethylamino, phenylamino, pyridinylamino, acetylamino, tert-butoxycarbonylamino and aminocarbonyl.

Definitive examples of specific substituents on the monocyclic ring of which M is the residue include thiazolyl, pyridinyl, pyrazinyl, hydroxyethyl, oxo, acetyl, propionyl, tert-butylcarbonyl, hydroxyacetyl, (dimethylamino)acetyl, cyclopropylcarbonyl, thienylcarbonyl, pyridinylcarbonyl, carboxy, carboxymethyl, methoxycarbonyl, ethoxycarbonylmethyl, hydroxyacetylamino, acetylaminomethyl, cyclopropylcarbonyl-amino, aminocarbonyl, methylaminocarbonyl and dimethylaminocarbonylmethyl.

A particular substituent on the monocyclic ring of which M is the residue is oxo.

Selected values of the monocyclic ring of which M is the residue include pyrrolidin-1-yl, oxopyrrolidin-1-yl, hydroxyacetylaminopyrrolidin-1-yl, acetylamino-methylpyrrolidin-1-yl, cyclopropylcarbonylaminopyrrolidin-1-yl, oxooxazolidin-3-yl, carboxypiperidin-1-yl, aminocarbonylpiperidin-1-yl, methylaminocarbonylpiperidin-1-yl, thiazolylpiperazin-1-yl, pyridinylpiperazin-1-yl, pyrazinylpiperazin-1-yl, hydroxyethyl-piperazin-1-yl, oxopiperazin-1-yl, acetylpiperazin-1-yl, propionylpiperazin-1-yl, tert-butylcarbonylpiperazin-1-yl, hydroxyacetylpiperazin-1-yl, (dimethylamino)acetyl-piperazin-1-yl, cyclopropylcarbonylpiperazin-1-yl, thienylcarbonylpiperazin-1-yl, pyridinylcarbonylpiperazin-1-yl, carboxymethylpiperazin-1-yl, methoxycarbonyl-piperazin-1-yl, ethoxycarbonylmethylpiperazin-1-yl, aminocarbonylpiperazin-1-yl, methylaminocarbonylpiperazin-1-yl and dimethylaminocarbonylmethylpiperazin-1-yl.

Particular values of the monocyclic ring of which M is the residue include pyrrolidin-1-yl, morpholin-4-yl and 3-oxopiperazin-1-yl.

One specific value of the monocyclic ring of which M is the residue is 3-oxopiperazin-1-yl.

Alternatively, the ring of which M is the residue may be optionally fused to an optionally substituted heteroaromatic ring. Suitably, this will be a five-membered or six-membered heteroaromatic ring, either of which may be optionally substituted by one or more substituents. Suitable five-membered heteroaromatic rings include furyl, thienyl, pyrrolyl, pyrazolyl, oxazolyl, isoxazolyl, thiazolyl, isothiazolyl, imidazolyl, oxadiazolyl, thiadiazolyl, triazolyl and tetrazolyl. Suitable six-membered heteroaromatic rings include pyridinyl, pyridazinyl, pyrimidinyl, pyrazinyl and triazinyl.

Suitably, the ring of which M is the residue may be optionally fused to an optionally substituted triazolyl ring, especially [1,2,4]triazolyl.

The heteroaromatic ring may be unsubstituted, or substituted, where possible, by one or more substituents, typically by one or two substituents. In one embodiment, the heteroaromatic ring is unsubstituted. In another embodiment, the heteroaromatic ring is monosubstituted. In a further embodiment, the heteroaromatic ring is disubstituted. Examples of suitable substituents on the heteroaromatic ring include C₁₋₆ alkyl, C₃₋₇ cycloalkyl, aryl, aryl(C₁₋₆)alkyl, C₃₋₇ heterocycloalkyl, heteroaryl, heteroaryl(C₁₋₆)alkyl, C₁₋₆ alkoxy, C₁₋₆ alkylthio, amino, C₁₋₆ alkylamino, di(C₁₋₆)alkylamino, halogen, cyano and trifluoromethyl.

A particular substituent on the heteroaromatic ring is C₁₋₆ alkyl, especially methyl.

Favourably, the ring of which M is the residue may be optionally fused to a triazolyl or methyltriazolyl ring, especially [1,2,4]triazolyl or 3-methyl[1,2,4]triazolyl.

In this context, the moiety of which M is the residue is suitably 5,6-dihydro-8H-[1,2,4]triazolo[4,3-a]pyrazin-7-yl or 3-methyl-5,6-dihydro-8H-[1,2,4]triazolo[4,3-a]-pyrazin-7-yl.

Typical values of R¹, R² and/or R³ include hydrogen, halogen, C₁₋₆ alkyl, aryl(C₁₋₆)alkyl and C₁₋₆ alkoxy.

Selected values of R¹, R² and/or R³ include hydrogen, halogen and C₁₋₆ alkyl.

Suitably, R¹, R² and R³ independently represent hydrogen, fluoro, chloro, bromo, cyano, nitro, methyl, ethyl, trifluoromethyl, benzyl, hydroxy, methoxy, difluoromethoxy, trifluoromethoxy, methylthio, methylsulfinyl, methylsulfonyl, amino, methylamino, dimethylamino, acetylamino, methoxycarbonylamino, methylsulfonylamino, formyl, acetyl, carboxy, methoxycarbonyl, aminocarbonyl, methylaminocarbonyl, dimethylaminocarbonyl, aminosulfonyl, methylaminosulfonyl or dimethylaminosulfonyl.

Typically, R¹ represents hydrogen, halogen, C₁₋₆ alkyl, aryl(C₁₋₆)alkyl or C₁₋₆ alkoxy.

Selected values of R¹ include hydrogen, halogen and C₁₋₆ alkyl.

Suitably, R¹ represents hydrogen or C₁₋₆ alkyl, typically methyl.

In one embodiment, R¹ represents hydrogen. In another embodiment, R¹ represents halogen, particularly fluoro or chloro. In one aspect of that embodiment, R¹ represents fluoro. In another aspect of that embodiment, R¹ represents chloro. In a further embodiment, R¹ represents C₁₋₆ alkyl, particularly methyl or ethyl. In one aspect of that embodiment, R¹ represents methyl. In another aspect of that embodiment, R¹ represents ethyl. In a still further embodiment, R¹ represents aryl(C₁₋₆)alkyl, especially benzyl. In an additional embodiment, R¹ represents C₁₋₆ alkoxy, especially methoxy.

Typically, R² represents hydrogen or halogen.

In one embodiment, R² represents hydrogen. In another embodiment, R² represents halogen, particularly fluoro or chloro. In one aspect of that embodiment, R² represents fluoro. In another aspect of that embodiment, R² represents chloro.

Typically, R³ represents hydrogen.

In a particular embodiment, R² and R³ both represent hydrogen.

Typical examples of suitable substituents on R⁴ and/or R⁵ and/or R⁶ and/or R⁷ include halogen, C₁₋₆ alkyl, C₁₋₆ alkoxy, difluoromethoxy, trifluoromethoxy, C₁₋₆ alkoxy(C₁₋₆)alkyl, C₁₋₆ alkylthio, C₁₋₆ alkylsulphonyl, hydroxy, hydroxy(C₁₋₆)alkyl, amino(C₁₋₆)alkyl, cyano, trifluoromethyl, oxo, C₂₋₆ alkylcarbonyl, carboxy, C₂₋₆ alkoxycarbonyl, amino, C₁₋₆ alkylamino, di(C₁₋₆)alkylamino, phenylamino, pyridinylamino, C₂₋₆ alkylcarbonylamino, C₂₋₆ alkoxycarbonylamino and aminocarbonyl.

Typical examples of specific substituents on R⁴ and/or R⁵ and/or R⁶ and/or R⁷ include fluoro, chloro, bromo, methyl, ethyl, isopropyl, methoxy, isopropoxy, difluoromethoxy, trifluoromethoxy, methoxymethyl, methylthio, ethylthio, methylsulphonyl, hydroxy, hydroxymethyl, hydroxyethyl, aminomethyl, cyano, trifluoromethyl, oxo, acetyl, carboxy, methoxycarbonyl, ethoxycarbonyl, tert-butoxycarbonyl, amino, methylamino, ethylamino, dimethylamino, phenylamino, pyridinylamino, acetylamino, tert-butoxycarbonylamino and aminocarbonyl.

Typical values of R⁴ include hydrogen, C₁₋₆ alkyl, —SR^{a}, —SO₂R^{a} and —NR^{b}R^{c}.

Selected values of R⁴ include hydrogen, —OR^{a}, —SR^{a}, —SOR^{a}, —SO₂R^{a} and —NR^{b}R^{c}.

Suitable values of R⁴ include hydrogen, —SR^{a} and —SO₂R^{a}.

In one embodiment, R⁴ represents hydrogen. In another embodiment, R⁴ represents C₁₋₆ alkyl, especially methyl. In another embodiment, R⁴ represents —OR^{a}. In a further embodiment, R⁴ represents —SR^{a}. In a further embodiment, R⁴ represents —SOR^{a}. In a further embodiment, R⁴ represents —SO₂R^{a}. In an additional embodiment, R⁴ represents —NR^{b}R^{c}.

Suitable values of R⁵ include hydrogen, C₁₋₆ alkyl and —NR^{b}R^{c}.

In one embodiment, R⁵ represents hydrogen. In another embodiment, R⁵ represents C₁₋₆ alkyl, especially methyl. In a further embodiment, R⁵ represents —NR^{b}R^{c}.

Suitable values of R⁶ include hydrogen, C₁₋₆ alkyl and —NR^{b}R^{c}.

In one embodiment, R⁶ represents hydrogen. In another embodiment, R⁶ represents C₁₋₆ alkyl, especially methyl. In a further embodiment, R⁶ represents —NR^{b}R^{c}.

Suitable values of R⁷ include hydrogen and C₁₋₆ alkyl.

In one embodiment, R⁷ represents hydrogen. In another embodiment, R⁷ represents C₁₋₆ alkyl, especially methyl.

In one embodiment, R⁸ represents hydrogen. In another embodiment, R⁸ represents C₁₋₆ alkyl, especially methyl.

Suitable values of the group R⁸ include hydrogen and methyl.

Typically, R⁹ represents hydrogen or C₁₋₆ alkyl.

In one embodiment, R⁹ represents hydrogen. In another embodiment, R⁹ represents halogen, particularly fluoro or chloro. In one aspect of that embodiment, R⁹ represents fluoro. In another aspect of that embodiment, R⁹ represents chloro. In a further embodiment, R⁹ represents C₁₋₆ alkyl, especially methyl. In an additional embodiment, R⁹ represents C₁₋₆ alkoxy, especially methoxy.

Suitable values of the group R⁹ include hydrogen, fluoro, chloro, bromo, methyl and methoxy. Suitably, R⁹ represents hydrogen or methyl. Typically, R⁹ represents hydrogen.

In one embodiment, R^{a} represents C₁₋₆ alkyl, especially methyl. In another embodiment, R^{a} represents difluoromethyl. In a further embodiment, R^{a} represents trifluoromethyl.

Typical values of R^{b} include hydrogen and C₁₋₆ alkyl.

Illustratively, R^{b} represents hydrogen or trifluoromethyl; or methyl, ethyl, n-propyl, isopropyl, n-butyl, 2-methylpropyl, tert-butyl, pentyl, hexyl, cyclopropyl, cyclobutyl, cyclopentyl, cyclohexyl, cyclopropylmethyl, cyclobutylmethyl, cyclopentylmethyl, cyclohexylmethyl, phenyl, benzyl, phenylethyl, azetidinyl, tetrahydrofuryl, tetrahydrothienyl, pyrrolidinyl, piperidinyl, homopiperidinyl, morpholinyl, azetidinylmethyl, tetrahydrofurylmethyl, pyrrolidinylmethyl, pyrrolidinylethyl, pyrrolidinylpropyl, thiazolidinylmethyl, imidazolidinylethyl, piperidinylmethyl, piperidinylethyl, tetrahydroquinolinylmethyl, piperazinylpropyl, morpholinylmethyl, morpholinylethyl, morpholinylpropyl, pyridinyl, indolylmethyl, pyrazolylmethyl, pyrazolylethyl, imidazolylmethyl, imidazolylethyl, benzimidazolylmethyl, triazolylmethyl, pyridinylmethyl or pyridinylethyl, any of which groups may be optionally substituted by one or more substituents.

Typical examples of suitable substituents on R^{b} include halogen, C₁₋₆ alkyl, C₁₋₆ alkoxy, difluoromethoxy, trifluoromethoxy, C₁₋₆ alkoxy(C₁₋₆)alkyl, C₁₋₆ alkylthio, C₁₋₆ alkylsulphonyl, hydroxy, hydroxy(C₁₋₆)alkyl, amino(C₁₋₆)alkyl, cyano, trifluoromethyl, oxo, C₂₋₆ alkylcarbonyl, carboxy, C₂₋₆ alkoxycarbonyl, amino, C₁₋₆ alkylamino, di(C₁₋₆)-alkylamino, phenylamino, pyridinylamino, C₂₋₆ alkylcarbonylamino, C₂₋₆ alkoxycarbonyl-amino and aminocarbonyl.

Typical examples of specific substituents on R^{b} include fluoro, chloro, bromo, methyl, ethyl, isopropyl, methoxy, isopropoxy, difluoromethoxy, trifluoromethoxy, methoxymethyl, methylthio, ethylthio, methylsulphonyl, hydroxy, hydroxymethyl, hydroxyethyl, aminomethyl, cyano, trifluoromethyl, oxo, acetyl, carboxy, methoxycarbonyl, ethoxycarbonyl, tert-butoxycarbonyl, amino, methylamino, ethylamino, dimethylamino, phenylamino, pyridinylamino, acetylamino, tert-butoxycarbonylamino and aminocarbonyl.

In one embodiment, R^{b} represents hydrogen. In another embodiment, R^{b} represents C₁₋₆ alkyl, especially methyl.

Suitably, R^{c} represents hydrogen or C₁₋₆ alkyl. In one embodiment, R^{c} is hydrogen.

In another embodiment, R^{c} represents C₁₋₆ alkyl, especially methyl or ethyl, particularly methyl. In a further embodiment, R^{c} represents C₃₋₇ cycloalkyl, e.g. cyclopropyl, cyclobutyl, cyclopentyl or cyclohexyl.

In one embodiment, R^{d} represents hydrogen. In another embodiment, R^{d} represents C₁₋₆ alkyl, especially methyl.

Suitably, R^{e} represents methyl.

One sub-class of compounds according to the invention is represented by the compounds of formula (IIA) and N-oxides thereof, and pharmaceutically acceptable salts and solvates thereof:

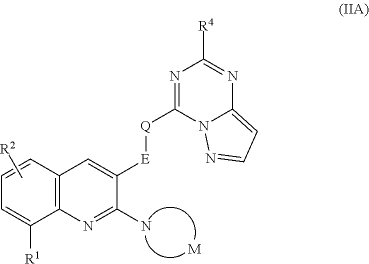

wherein E, Q, M, R¹, R² and R⁴ are as defined above.

Another sub-class of compounds according to the invention is represented by the compounds of formula (IIB) and N-oxides thereof, and pharmaceutically acceptable salts and solvates thereof:

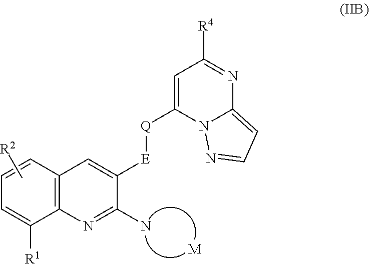

wherein E, Q, M, R¹, R² and R⁴ are as defined above.

Specific novel compounds in accordance with the present invention include each of the compounds whose preparation is described in the accompanying Examples, and pharmaceutically acceptable salts and solvates thereof.

The present invention also provides a pharmaceutical composition which comprises a compound in accordance with the invention as described above, or a pharmaceutically acceptable salt or solvate thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds of formula (I) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (I) may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds of use in the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds of use in the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds of use in the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compounds of use in the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound of use in the invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

The compounds of formula (I) above wherein Q represents oxygen, sulfur or N—R⁸ may be prepared by a process which comprises reacting a compound of formula (III) with a compound of formula (IV):

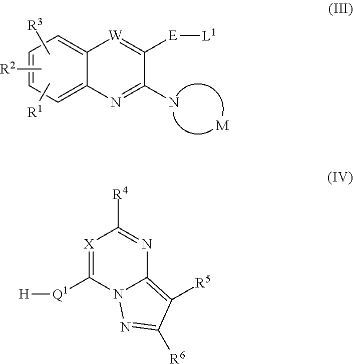

wherein L¹ represents a suitable leaving group, Q¹ represents oxygen, sulfur or N—R⁸, and X, E, M, W, R¹, R², R³, R⁴, R⁵, R⁶ and R⁸ are as defined above.

The leaving group L¹ is typically a halogen atom, e.g. bromo or iodo.

The reaction is conveniently effected at ambient or elevated temperature in a suitable solvent, e.g. N,N-dimethylformamide or acetonitrile. The reaction may be performed in the presence of a suitable base, e.g. an inorganic base such as potassium carbonate, cesium carbonate, sodium hydride or aqueous sodium hydroxide.

The intermediates of formula (III) above wherein L¹ is bromo or iodo may be prepared from a compound of formula (V):

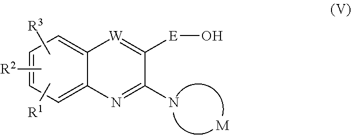

wherein E, M, W, R¹, R² and R³ are as defined above; by bromination or iodination.

The bromination reaction is conveniently effected by stirring compound (V) with an appropriate brominating agent, e.g. phosphorus tribromide, in a suitable solvent, e.g. a halogenated hydrocarbon such as dichloromethane.

The iodination reaction is conveniently effected by stirring compound (V) with an appropriate iodinating agent, e.g. elemental iodine, in a suitable solvent, e.g. a halogenated hydrocarbon such as dichloromethane, typically in the presence of triphenylphosphine and imidazole.

Alternatively, the intermediates of formula (III) above wherein E represents methylene and L¹ is bromo may be prepared from a compound of formula (VI):

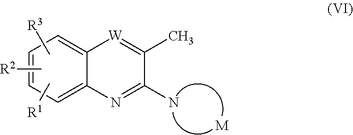

wherein M, W, R¹, R² and R³ are as defined above; by bromination.

The reaction is conveniently effected at an elevated temperature in a suitable solvent, e.g. a halogenated solvent such as carbon tetrachloride, in the presence of a suitable brominating agent, e.g. N-bromosuccinimide, typically in the presence of a catalyst such as benzoyl peroxide.

In another procedure, the compounds of formula (I) wherein Q represents oxygen may be prepared by a process which comprises reacting a compound of formula (V) as defined above with a compound of formula (VII):

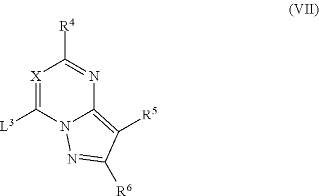

wherein X, R⁴, R⁵ and R⁶ are as defined above, and L² represents a suitable leaving group.

The leaving group L² is typically a halogen atom, e.g. chloro.

The reaction is conveniently effected by stirring compounds (V) and (VII) in a suitable solvent, e.g. N,N-dimethylformamide, typically under basic conditions, e.g. in the presence of an inorganic base such as sodium hydride.

In another procedure, the compounds of formula (I) wherein Q represents sulfur may be prepared by a process which comprises reacting a compound of formula (VII) as defined above with a compound of formula (VIII):

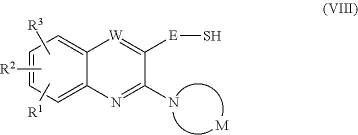

wherein E, M, W, R¹, R² and R³ are as defined above.

The reaction is conveniently effected by stirring compounds (VII) and (VIII) in a suitable solvent, e.g. a lower alkanol such as methanol, typically under basic conditions, e.g. in the presence of an alkali metal alkoxide such as sodium methoxide.

The intermediates of formula (VIII) may typically be prepared by treating a suitable compound of formula (III) above with thiolacetic acid; followed by treatment of the resulting compound with a base, e.g. an alkali metal alkoxide such as sodium methoxide.

In another procedure, the compounds of formula (I) wherein Q represents N—R⁸ may be prepared by a process which comprises reacting a compound of formula (VII) as defined above with a compound of formula (IX):

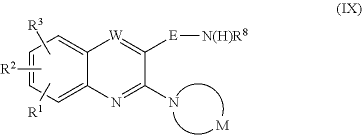

wherein E, M, W, R¹, R², R³ and R⁸ are as defined above.

The reaction is conveniently effected at ambient or elevated temperature in a suitable solvent, e.g. tetrahydrofuran, n-butanol, 1-methyl-2-pyrrolidinone (NMP) or dichloromethane. The reaction may be performed in the presence of a suitable base, e.g. an organic base such as N,N-diisopropylethylamine.

The intermediates of formula (IX) wherein R⁸ represents hydrogen may be prepared by treating a suitable compound of formula (III) above with potassium phthalimide; followed by treatment of the resulting compound with hydrazine. Alternatively, they may be prepared by treating a suitable compound of formula (III) above with sodium azide; followed by treatment of the resulting compound with triphenylphosphine.

In an additional procedure, the compounds of formula (I) wherein E represents methylene and Q represents N—R⁸ may be prepared by a process which comprises reacting a compound of formula (X) with a compound of formula (XI):

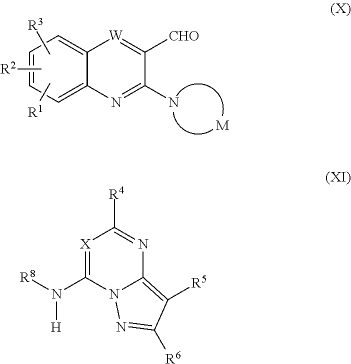

wherein X, M, W, R¹, R², R³, R⁴, R⁵, R⁶ and R⁸ are as defined above; under reducing conditions.

The reaction is conveniently effected by stirring compounds (X) and (XI) at an elevated temperature in a suitable solvent, e.g. a cyclic ether such as tetrahydrofuran, in the presence of a reducing agent. A suitable reducing agent comprises a mixture of di-n-butyltin dichloride and phenylsilane.

The intermediates of formula (IX) wherein E represents methylene and R⁸ represents C₁₋₆ alkyl, e.g. methyl, may be prepared by treating a suitable compound of formula (X) above with a C₁₋₆ alkylamine, e.g. methylamine, in the presence of titanium(IV) n-propoxide and a base, e.g. an organic base such as N,N-diisopropylamine; followed by treatment of the resulting compound with a reducing agent, e.g. sodium triacetoxyborohydride.

The intermediates of formula (IX) wherein E represents (methyl)methylene and R⁸ represents hydrogen may be prepared by a three-step procedure which comprises: (i) treating a suitable compound of formula (X) above with 2-methyl-2-propanesulfinamide in the presence of titanium(IV) isopropoxide; (ii) reaction of the resulting compound with a Grignard reagent, e.g. methylmagnesium bromide; and (iii) treatment of the resulting compound with a mineral acid, e.g. hydrochloric acid.

Similarly, the intermediates of formula (IX) wherein E represents methylene and R⁸ represents hydrogen may be prepared by a three-step procedure which comprises: (i) treating a suitable compound of formula (X) above with 2-methyl-2-propanesulfinamide in the presence of titanium(IV) isopropoxide; (ii) reaction of the resulting compound with a reducing reagent, e.g. sodium borohydride; and (iii) treatment of the resulting compound with a mineral acid, e.g. hydrochloric acid.

The intermediates of formula (V) wherein E represents methylene may be prepared from the corresponding compound of formula (X) by treatment with a reducing agent, e.g. sodium borohydride.

The intermediates of formula (V), (VIII) and (IX) may be prepared by reacting a compound of formula (XII) with a compound of formula (XIII):

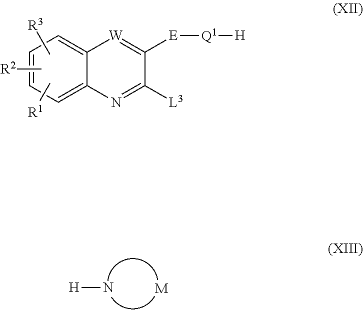

wherein E, Q¹, M, W, R¹, R² and R³ are as defined above, and L³ represents a suitable leaving group.

The leaving group L³ is typically a halogen atom, e.g. chloro.

The reaction is conveniently effected at an elevated temperature in a suitable solvent, e.g. tetrahydrofuran, n-butanol or 1-methyl-2-pyrrolidinone (NMP). The reaction may be performed in the presence of a suitable base, e.g. an organic base such as N,N-diisopropylethylamine

The intermediates of formula (XII) wherein E represents (methyl)methylene and Q¹ represents NH may be prepared by a three-step procedure which comprises: (i) treating a suitable compound of formula (XIV):

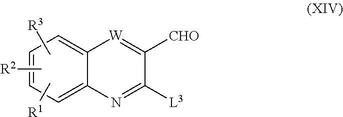

wherein W, R¹, R², R³ and L³ are as defined above; with 2-methyl-2-propanesulfinamide in the presence of titanium(IV) isopropoxide; (ii) reaction of the resulting compound with a Grignard reagent, e.g. methylmagnesium bromide; and (iii) treatment of the resulting compound with a mineral acid, e.g. hydrochloric acid.

Similarly, the intermediates of formula (XII) wherein E represents methylene and Q¹ represents NH may be prepared by a three-step procedure which comprises: (i) treating a suitable compound of formula (XIV) above with 2-methyl-2-propanesulfinamide in the presence of titanium(IV) isopropoxide; (ii) reaction of the resulting compound with a reducing reagent, e.g. sodium borohydride; and (iii) treatment of the resulting compound with a mineral acid, e.g. hydrochloric acid.

In a further procedure, the compounds of formula (I) may be prepared by a process which comprises reacting a compound of formula (XIII) as defined above with a compound of formula (XV):

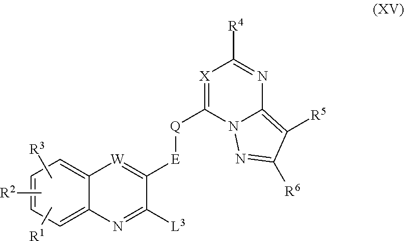

wherein X, E, Q, W, R¹, R², R³, R⁴, R⁵, R⁶ and L³ are as defined above.

The reaction is conveniently effected in the presence of a transition metal catalyst. Suitably, the transition metal catalyst may be a copper(I) salt, e.g. a copper(I) halide such as copper(I) iodide, in which case the reaction is conveniently performed in the presence of a suitable base, e.g. a phosphate salt such as potassium phosphate, and a catalytic quantity of N,N′-dimethylcyclohexane-1,2-diamine. Alternatively, the transition metal catalyst may be a palladium complex, e.g. tris(dibenzylideneacetone)dipalladium(0), in which case the reaction is conveniently performed in the presence of 1,2,3,4,5-pentaphenyl-1′-(di-tert-butylphosphino)ferrocene (Q-Phos) and a suitable base, e.g. a lower alkoxide salt such as potassium tert-butoxide.

Moreover, the reaction may be conveniently carried out at an elevated temperature in a suitable solvent, e.g. a hydrocarbon solvent such as toluene.

The intermediates of formula (XV) wherein Q represents oxygen, sulfur or N—R⁸ may be prepared by reacting a compound of formula (XII) as defined above with a compound of formula (VII) as defined above, under conditions analogous to those described above for the reaction of a compound of formula (V), (VIII) or (IX) with a compound of formula (VII).

Where they are not commercially available, the starting materials of formula (IV), (VI), (VII), (X), (XI), (XIII) and (XIV) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of illustration, a compound of formula (I) wherein R⁴ represents —SR^{a} may be converted into the corresponding compound wherein R⁴ represents —SOR^{a} or —SO₂R^{a} by treatment with an oxidising agent, e.g. 3-chloroperoxybenzoic acid. A compound of formula (I) wherein R⁴ represents —SO₂R^{a} may be converted into the corresponding compound wherein R⁴ represents hydrogen by treatment with a reducing agent, e.g. sodium borohydride. A compound of formula (I) wherein R⁴ represents —SO₂R^{a} may be converted into the corresponding compound wherein R⁴ represents —OR^{a} by treatment with an alcohol of formula R^{a}—OH. A compound of formula (I) wherein R⁴ represents —SO₂R^{a} may be converted into the corresponding compound wherein R⁴ represents —NR^{b}R^{c} by treatment with an amine of formula H—NR^{b}R^{c} or a salt thereof, e.g. ammonium hydroxide.

A compound of formula (I) substituted by an alkoxycarbonyl group, e.g. methoxycarbonyl or ethoxycarbonyl, may be converted into the corresponding compound substituted by carboxy by treatment with a base, suitably an inorganic base, e.g. an alkali metal hydroxide such as lithium hydroxide or sodium hydroxide.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} edition, 1999. The protecting groups may be removed at any convenient subsequent stage utilising methods known from the art.

The following Examples illustrate the preparation of compounds according to the invention.

The compounds in accordance with this invention potently inhibit the activity of human PI3Kα and/or PI3Kβ and/or PI3Kγ and/or PI3Kδ.

### Enzyme Inhibition Assays

Measurement of the ability of compounds to inhibit the lipid kinase activity of the four class 1 PI3 kinase isoforms (α, β, γ and δ) was performed using a commercially available homogeneous time-resolved fluorescence assay as described by Gray et al., Anal. Biochem., 2003, 313, 234-245, according to the manufacturer's instructions (Upstate). All assays were performed at 2 μM ATP and a concentration of purified class 1 PI3 kinase known to generate product within the linear range of the assay. Dilutions of inhibitor in DMSO were added to the assay and compared with assays run in the presence of 2% (v/v) DMSO alone (100% activity). The concentration of inhibitor required to inhibit the enzyme activity by 50% is quoted as the IC₅₀.

When tested in the above assay, the compounds of the accompanying Examples were all found to possess IC₅₀ values for inhibition of activity of human PI3Kα and/or PI3Kβ and/or PI3Kγ and/or PI3Kδ of 50 μM or better.

### EXAMPLES

### Abbreviations

| | |
|---|---|
| DCM: dichloromethane | DIPEA: N,N-diisopropylethylamine |
| DMSO: dimethylsulfoxide | Et₂O: diethyl ether |
| EtOAc: ethyl acetate | EtOH: ethanol |
| MeCN: acetonitrile | MeOH: methanol |
| NMP: 1-methyl-2-pyrrolidinone | THF: tetrahydrofuran |
| n-BuOH: n-butanol | TFA: trifluoroacetic acid |
| DMAP: 4-(dimethylamino)pyridine | |

Q-Phos: 1,2,3,4,5-pentaphenyl-1′-(di-tert-butylphosphino)ferrocene

| | |
|---|---|
| h: hour | r.t.: room temperature |
| RT: retention time | SiO₂: silica |
| br: broad | M: mass |
| HPLC: High Performance Liquid Chromatography | |
| LCMS: Liquid Chromatography Mass Spectrometry | |
| ES+: Electrospray Positive Ionisation | |

DCM: dichloromethane DIPEA: N,N-diisopropylethylamine
DMSO: dimethylsulfoxide Et₂O: diethyl ether
EtOAc: ethyl acetate EtOH: ethanol
MeCN: acetonitrile MeOH: methanol
NMP: 1-methyl-2-pyrrolidinone THF: tetrahydrofuran
n-BuOH: n-butanol TFA: trifluoroacetic acid
DMAP: 4-(dimethylamino)pyridine
Q-Phos: 1,2,3,4,5-pentaphenyl-1′-(di-tert-butylphosphino)ferrocene
h: hour r.t.: room temperature
RT: retention time SiO₂: silica
br: broad M: mass

### HPLC: High Performance Liquid Chromatography

### LCMS: Liquid Chromatography Mass Spectrometry

### ES+: Electrospray Positive Ionisation

### Analytical Conditions

All NMRs were obtained at 400 MHz.

Compounds were named with the aid of the Cambridgesoft Chemistry Cartridge (v. 9.0.0.182) software.

All reactions involving air- or moisture-sensitive reagents were performed under a nitrogen atmosphere using dried solvents and glassware. Degassing was performed by bubbling nitrogen through the reaction mixture.

Analytical methods used for LCMS were Methods 1 and 2 below.

| Method | Description | | |
|---|---|---|---|
| 1 | Solvents: | Acetonitrile (far UV grade) | |
| | | Water (high purity via PureLab Option unit) | |
| | | with 10 mM ammonium hydrogencarbonate | |
| | Column: | Waters Xterra MS 5 μm C18, 100 × 4.6 mm | |
| | | (Plus guard cartridge) | |
| | Flow Rate: | 2 mL/min | |
| | Gradient: | A: Water/Bicarb | B: MeCN |
| | Time | A % | B % |
| | 0.00 | 95 | 5 |
| | 0.50 | 95 | 5 |
| | 4.00 | 5 | 95 |
| | 5.50 | 5 | 95 |
| | 5.60 | 95 | 5 |
| | 6.50 | 95 | 5 |
| 2 | Solvents: | Acetonitrile (far UV grade) with 0.1% (v/v) | |
| | | formic acid | |
| | | Water (high purity via PureLab Option unit) | |
| | | with 0.1% formic acid | |
| | Column: | Phenomenex Luna 5 μm C18(2), 100 × | |
| | | 4.6 mm (Plus guard cartridge) | |
| | Flow Rate: | 2 mL/min | |
| | Gradient: | A: Water/ | B: MeCN/ |
| | | formic acid | formic acid |
| | Time | A % | B % |
| | 0.00 | 95 | 5 |
| | 3.50 | 5 | 95 |
| | 5.50 | 5 | 95 |
| | 5.60 | 95 | 5 |
| | 6.50 | 95 | 5 |

### Intermediate 1

### N-[(2,8-Dichloroquinolin-3-yl)methylene]-2-methylpropane-2(R)-sulfinamide

Titanium isopropoxide (26.5 mL, 88.5 mmol) was added in a single portion to a stirred solution of 2,8-dichloroquinoline-3-carboxaldehyde (10 g, 44.25 mmol) in anhydrous THF (100 mL) and the mixture was stirred at r.t. for 15 minutes. (R)-(+)-2-Methyl-2-propanesulfinamide (5.36 g, 44.25 mmol) was added in a single portion and the mixture was stirred at r.t. for 17 h. Water (250 mL) was added and a precipitate was obtained. This was filtered and washed with DCM (4×200 mL). The organic layer was separated, dried (MgSO₄) and the solvent removed in vacuo to afford the title compound (13.0 g, 89%) as a pale yellow solid. δ_{H} (CDCl₃) 9.12 (s, 1H), 8.83 (s, 1H), 7.93 (dd, J 7.5, 1.3 Hz, 1H), 7.88 (dd, J 8.2, 1.3 Hz, 1H), 7.50-7.59 (m, 1H), 1.33 (s, 9H).

### Intermediate 2

### N—[(S)-1-(2,8-Dichloroquinolin-3-yl)ethyl]-2-methylpropane-2(R)-sulfinamide

Methylmagnesium bromide (26.4 mL, 79 mmol; 3M in Et₂O) was added dropwise over 10 minutes to a stirred solution of Intermediate 1 (13 g, 39.5 mmol) in DCM (300 mL) at −70° C. After complete addition, the reaction mixture was gradually allowed to reach r.t. whilst stirring overnight. Saturated aqueous NH₄Cl (200 mL) was added and the aqueous layer was extracted with DCM (200 mL). The combined organics were dried (MgSO₄), filtered and the solvent was removed in vacuo. The resulting gum was triturated with Et₂O to afford the title compound (5.83 g, 42%) as a colourless solid. δ_{H} (CDCl₃) 8.25 (s, 1H), 7.83 (d, J 7.5 Hz, 1H), 7.74 (d, J 8.1 Hz, 1H), 7.49 (t, J 7.8 Hz, 1H), 5.05-5.20 (m, 1H), 3.46 (d, J 4.9 Hz, 1H), 1.71 (d, J 6.7 Hz, 3H), 1.25 (s, 9H).

### Intermediate 3

### (S)-1-(2,8-Dichloroquinolin-3-yl)ethanamine, hydrochloric acid salt

HCl (7 mL, 28 mmol; 4N in 1,4-dioxane) was added to a stirred solution of Intermediate 2 (4.53 g, 13.96 mmol) in MeOH (30 mL) and the reaction mixture was stirred at r.t. for 2 h. The mixture was concentrated under reduced pressure to give the title compound (4.3 g) as a pale yellow gum, which was used in the next step without further purification. δ_{H} (DMSO-d₆) 8.80-9.00 (br m, 4H), 8.10 (dd, J 7.5, 1.3 Hz, 1H), 8.05 (dd, J 8.2, 1.3 Hz, 1H), 7.70-7.80 (m, 1H), 4.86 (m, 1H), 1.70 (d, J 6.7 Hz, 3H).

### Intermediate 4

### (S)-tert-Butyl 1-(2,8-dichloroquinolin-3-yl)ethylcarbamate

Di-tert-butyl dicarbonate (6.09 g, 27.9 mmol) was added to a stirred solution of Intermediate 3 (4.3 g) and DIPEA (12 mL, 69.4 mmol) in DCM (200 mL). The resulting mixture was allowed to stand at r.t. for 66 h. The mixture was washed with saturated aqueous NaHCO₃ (50 mL) and the organic layer was separated, dried (MgSO₄), and the solvent was removed in vacuo. The residue was triturated with 40-60° petroleum ether and the resulting solid was dried under vacuum to afford the title compound (4.03 g, 90% from Intermediate 2) as a pale pink solid. δ_{H} (CDCl₃) 8.13 (s, 1H), 7.81 (d, J 7.5 Hz, 1H), 7.73 (dd, J 8.2, 1.3 Hz, 1H), 7.47 (t, J 7.8, 1H), 4.70-5.23 (m, 2H), 1.10-1.65 (m, 12H).

### Intermediate 5

### (S)-tert-Butyl 1-[8-chloro-2-(3-oxopiperazin-1-yl)quinolin-3-yl]ethylcarbamate

To a solution of Intermediate 4 (1.0 g, 3.1 mmol) in NMP (5 mL) and DIPEA (2 mL, 11.0 mmol) was added 2-oxopiperazine (1.56 g, 15.6 mmol). The resulting mixture was heated at 140° C. After 15 h, the solvents were removed in vacuo. The residue was redissolved in DCM (20 mL) and washed with water (2×10 mL). The organic layer was dried (MgSO₄), and the solvent was removed in vacuo. The residue was purified by column chromatography (SiO₂, 0-100% EtOAc in isohexane) to give the title compound (520 mg, 41%) as a solid. δ_{H} (CDCl₃) 8.03 (s, 1H), 7.72 (dd, J 7.5, 1.3 Hz, 1H), 7.63 (dd, J 8.07, 1.3 Hz, 1H), 7.23-7.36 (m, 1H), 6.32 (s, 1H), 4.93-5.15 (m, 2H), 4.36 (d, J 17.7 Hz, 1H), 3.84-4.13 (m, 2H), 3.68-3.77 (m, 1H), 3.30-3.59 (m, 2H), 1.41-1.47 (m, 12H).

### Intermediate 6

### (E)-N-[(2-Chloro-8-methylquinolin-3-yl)methylene]-(R)-2-methylpropane-2-sulfinamide

Following the procedure described for Intermediate 1,2-chloro-8-methyl-quinoline-3-carboxaldehyde (2.05 g, 10 mmol), titanium isopropoxide (5.68 g, 20 mmol) and (R)-(+)-2-methyl-2-propanesulfinamide (1.21 g, 10 mmol) afforded the title compound (2.4 g, 72%) as a pale yellow solid. δ_{H} (CDCl₃) 9.12 (s, 1H), 8.79 (s, 1H), 7.78 (d, J 8.4 Hz, 1H), 7.67 (d, J 6.8 Hz, 1H), 7.51 (t, J 7.8 Hz, 1H), 2.79 (s, 3H), 1.32 (s, 9H). LCMS (ES+) 309, 311 (M+H)⁺.

### Intermediate 7

### N—[(S)-1-(2-Chloro-8-methylquinolin-3-yl)ethyl]-(R)-2-methylpropane-2-sulfinamide

Following the procedure described for Intermediate 2, Intermediate 6 (1.9 g, 6.15 mmol) and methylmagnesium bromide (4.1 mL, 12.3 mmol, 3.0M in Et₂O) afforded, after crystallisation from 40-60 petroleum ether, the title compound (900 mg, 45%) as a pale yellow solid. δ_{H} (CDCl₃) 8.17 (s, 1H), 7.64 (d, J 8.0 Hz, 1H), 7.56 (d, J 7.2 Hz, 1H), 7.45 (t, J 7.6 Hz, 1H), 5.09-5.12 (m, 1H), 3.44 (d, J 4.8 Hz, 1H), 2.77 (s, 3H), 1.71 (d, J 6.8 Hz, 3H), 1.25 (s, 9H). LCMS (ES+) 325, 327 (M+H)⁺.

### Intermediate 8

### (S)-1-(2-Chloro-8-methylquinolin-3-yl)ethanamine

To a solution of Intermediate 7 (0.25 g, 0.77 mmol) in MeOH (2 mL) was added conc. HCl (1 mL) and the mixture stirred at r.t. for 2 h. The reaction was poured into DCM (100 mL) and washed with 2M NaOH solution (50 mL). The organic layer was separated, dried (MgSO₄), filtered and the solvent removed in vacuo to afford the title compound (0.16 g, 94%) as a white solid. δ_{H} (CDCl₃) 8.29 (s, 1H), 7.66 (d, J 8.4 Hz, 1H), 7.53 (d, J 6.8 Hz, 1H), 7.41-7.45 (m, 1H), 4.61-4.67 (m, 1H), 2.76 (s, 3H), 1.50 (d, J 4.0 Hz, 3H). LCMS (ES+) 221, 223 (M+H)⁺.

### Intermediate 9

### (S)-tert-Butyl 1-(2-chloro-8-methylquinolin-3-yl)ethylcarbamate

Following the procedure described for Intermediate 4, Intermediate 8 (1.83 g, 3.11 mmol), DIPEA (2.7 mL, 15.6 mL) and di-tert-butyl dicarbonate (2.95 g, 4.12 mmol) afforded, after column chromatography (SiO₂, 0-30% EtOAc in petrol 40-60), the title compound (897 mg, 34%) as a white solid. δ_{H} (CDCl₃) 8.07 (s, 1H), 7.64 (d, J 8.0 Hz, 1H), 7.54 (d, J 7.2 Hz, 1H), 7.43 (t, J 7.6 Hz, 1H), 5.12-5.22 (m, 1H), 5.00-5.10 (m, 1H), 2.76 (s, 3H), 1.50-1.57 (m, 3H), 1.30-1.50 (m, 9H). LCMS (ES+) 321, 323 (M+H)⁺.

### Intermediate 10

### (E)-N-[(2-Chloro-7-fluoroquinolin-3-yl)methylene]-(R)-2-methylpropane-2-sulfinamide

Following the procedure described for Intermediate 1,2-chloro-7-fluoroquinoline-3-carboxaldehyde (6.3 g, 30 mmol), titanium(IV) isopropoxide (17.0 g, 60 mmol) and (R)-(+)-2-methyl-2-propanesulfinamide (3.6 g, 30 mmol) afforded the title compound (7.2 g, 76%) as a pale yellow solid. δ_{H} (CDCl₃) 9.09 (1H, s), 8.83 (1H, s), 7.96 (1H, dd, J 6.0 Hz,), 7.69 (1H, d, J 7.2 Hz), 7.42 (1H, t, J 8.4 Hz), 1.32 (9H, s).

### Intermediate 11

### N—[(S)-1-(2-Chloro-7-fluoroquinolin-3-yl)ethyl]-(R)-2-methylpropane-2-sulfinamide

Following the procedure described for Intermediate 2, Intermediate 10 (7.2 g, 23.5 mmol) and methylmagnesium bromide (16.0 mL, 48 mmol; 3.0M in Et₂O) afforded, after crystallisation from 40-60 petroleum ether, the title compound (4.0 g, 53%) as a pale yellow solid. δ_{H} (CDCl₃) 8.23 (1H, s), 7.16 (1H, dd, J 6.0 Hz), 7.65 (1H, d, J 7.2 Hz), 7.46 (1H, t, J 8.4 Hz), 5.16 (1H, q, J 6.8 Hz), 3.45 (1H, br s), 1.71 (3H, d, J 6.8 Hz), 1.26 (9H, s).

### Intermediate 12

### (S)-tert-Butyl 1-(2-chloro-7-fluoroquinolin-3-yl)ethylcarbamate

To a solution of Intermediate 11 (4.0 g, 12.17 mmol) in MeOH (20 mL) was added conc. HCl (1 mL) and the mixture stirred at r.t. for 2 h. The reaction mixture was partitioned between DCM (100 mL) and 2M NaOH solution (50 mL). The organic layer was dried (MgSO₄) and filtered. To this filtrate was added DIPEA (3.0 mL, 15.0 mmol), followed by a solution of di-tert-butyl dicarbonate (3.0 g, 13.76 mmol) in DCM (10 mL) dropwise. The reaction mixture was stirred at r.t. for 3 h, then diluted with DCM (10 mL) and washed with saturated NaHCO₃ solution (15 mL) and brine (15 mL). The organic layer was dried (MgSO₄), concentrated in vacuo and purified by column chromatography (SiO₂, 0-30% EtOAc in 40-60 petroleum ether) to give the title compound (3.4 g, 86%) as a white solid. δ_{H} (CDCl₃) 8.23 (1H, s), 7.16 (1H, dd, J 6.0 Hz), 7.65 (1H, d, J 7.2 Hz), 7.46 (1H, t, J 8.4 Hz), 5.18 (1H, br q, J 6.8 Hz), 3.49 (1H, d, J 6.8 Hz), 1.54 (3H, d, J 6.8 Hz), 1.48 (9H, s).

### Intermediate 13

### (E)-N-[(2-Chloro-7-fluoro-8-methylquinolin-3-yl)methylene]-(R)-2-methylpropane-2-sulfinamide

Following the procedure described for Intermediate 1,2-chloro-7-fluoro-8-methylquinoline-3-carbaldehyde (6.6 g, 29.5 mmol), titanium(IV) isopropoxide (17 g, 60 mmol) and (R)-(+)-2-methyl-2-propanesulfinamide (3.6 g, 29.5 mmol) afforded the title compound (8.3 g, 86%) as a yellow solid. δ_{H} (CDCl₃) 9.12 (1H, s), 8.73 (1H, s), 7.71 (1H, dd, J 6.0 Hz), 7.40 (1H, t, J 8.2 Hz), 2.69 (3H, s), 1.32 (9H, s).

### Intermediate 14

N—[(S)-1-(2-Chloro-7-fluoro-8-methylquinolin-3-yl)ethyl]-(13)-2-methylpropane-2-sulfinamide

Following the procedure described for Intermediate 2, Intermediate 13 (8.3 g, 25.4 mmol) and methylmagnesium bromide (16.0 mL, 48 mmol; 3.0M in Et₂O) afforded, after crystallisation from 40-60 petroleum ether, the title compound (4.2 g, 48%) as a yellow solid. δ_{H} (CDCl₃) 8.17 (1H, s), 7.63 (1H, dd, J 6.0 Hz), 7.32 (1H, t, J 8.8 Hz), 5.16 (1H, q, J 6.8 Hz), 3.45 (1H, d, J 6.8 Hz), 2.66 (3H, s) 1.70 (3H, d, J 6.8 Hz), 1.26 (9H, s).

### Intermediate 15

### (S)-tert-Butyl 1-(2-chloro-7-fluoro-8-methylquinolin-3-yl)ethylcarbamate

Following the procedure described for Intermediate 12, Intermediate 14 (4.2 g, 12.2 mmol) and conc. HCl (1 mL), then di-tert-butyl dicarbonate (2.7 g, 12.2 mmol) and DIPEA (1.6 g, 12.2 mmol), gave the title compound (4.38 g, 90%) as a yellow solid. δ_{H} (CDCl₃) 8.07 (1H, s), 7.62 (1H, dd, J 6.0 Hz), 7.30 (1H, t, J 8.8 Hz), 5.17 (1H, m), 5.07 (1H, br s), 2.65 (3H, s) 1.54 (3H, d, J 6.4 Hz), 1.42 (9H, s).

### Intermediate 16

### (E)-N-[(2-Chloro-5,7-difluoroquinolin-3-yl)methylene]-(R)-2-methylpropane-2-sulfinamide

To a solution of 2-chloro-5,7-difluoroquinoline-3-carboxaldehyde (7.9 g, 34.7 mmol) in dry THF (200 mL) under nitrogen was added titanium isopropoxide (20 g, 70 mmol). The mixture was stirred at r.t. for 10 minutes and treated with (R)-(+)-2-methyl-2-propanesulfinamide (4.3 g, 34.7 mmol). Stirring at r.t. continued for 72 h. The mixture was treated with water (60 mL), and the resulting precipitate was filtered through Kieselguhr and washed extensively with DCM. The filtrate was extracted with DCM (2×150 mL). The combined organic layers were dried (MgSO₄) and concentrated, to afford the title compound (9.35 g, 82%) as a pale yellow solid. δ_{H} (CDCl₃) 9.09 (1H, s), 9.02 (1H, s), 7.55 (1H, d, J 9.6 Hz), 7.14 (1H, t, J 9.6 Hz), 1.32 (9H, s).

### Intermediate 17

### N—[(S)-1-(2-Chloro-5,7-difluoroquinolin-3-yl)ethyl]-(R)-2-methylpropane-2-sulfinamide

To a solution of Intermediate 16 (9.35 g, 28.3 mmol) in toluene (40 mL) at r.t. was added dropwise over 30 minutes a solution of methylmagnesium bromide (12.0 mL, 36 mmol; 3.0M in Et₂O). The reaction mixture was stirred for 18 h. Saturated aqueous NH₄Cl solution (50 mL) was added. The solids were filtered off through Kieselguhr and the filtrate was extracted with DCM (2×100 mL). The combined organic layers were dried (MgSO₄) and concentrated, to give a pale yellow solid. This was crystallised from 40-60 petroleum ether to afford the title compound (6.7 g, 68%) as a pale yellow solid. δ_{H} (CDCl₃) 8.35 (1H, s), 7.49 (1H, d, J 9.6 Hz), 7.08 (1H, t, J 9.6 Hz), 5.19 (1H, q, J 6.8 Hz), 5.05 (1H, d, J 6.8 Hz), 1.59 (9H, s,), 1.43 (3H, d, J 6.8 Hz).

### Intermediate 18

### (S)-tert-Butyl 1-(2-chloro-5,7-difluoroquinolin-3-yl)ethylcarbamate

To a solution of Intermediate 17 (6.7 g, 19.3 mmol) in MeOH (20 mL) was added conc. HCl (1 mL). The mixture was stirred at r.t. for 2 h, poured into DCM (100 mL) and washed with 2M NaOH solution (50 mL). The organic layer was separated, dried (MgSO₄) and concentrated in vacuo. The crude product was dissolved in dry DCM (40 mL) and treated with di-tert-butyl dicarbonate (4.3 g, 19.3 mmol) and DIPEA (3.0 mL, 15.0 mmol). The reaction mixture was stirred at r.t. for 16 h, diluted with DCM (10 mL) and washed with saturated aqueous NaHCO₃ solution (15 mL) and brine (15 mL). The organic layer was separated, dried (MgSO₄) and concentrated in vacuo. Purification of the crude residue by column chromatography (SiO₂, 0-30% EtOAc in 40-60 petroleum ether) afforded the title compound (5.2 g, 78%) as a white solid. δ_{H} (CDCl₃) 8.45 (1H, s), 7.48 (1H, d, J 9.6 Hz), 7.10 (1H, t, J 9.6 Hz), 5.05 (1H, q, J 6.4 Hz), 3.77 (1H, d, J 6.4 Hz), 1.68 (3H, d, J 6.4 Hz), 1.26 (9H, s).

### Intermediate 19

### (S)-tert-Butyl 1-[2-(4-acetylpiperazin-1-yl)-8-methylquinolin-3-yl]ethylcarbamate

Intermediate 9 (150 mg, 0.47 mmol), 1-acetylpiperazine (300 mg, 2.34 mmol), DIPEA (0.42 mL, 2.34 mmol) and NMP (3 mL) were combined in a sealed tube and heated to 140° C. for 48 h. After cooling, the reaction mixture was dissolved in a 1:1 mixture of EtOAc and Et₂O (100 mL) and washed with saturated brine (3×25 mL). The organic layer was dried (MgSO₄) and concentrated in vacuo. Purification by column chromatography (SiO₂, 0-100% EtOAc in isohexane) afforded the title compound (151 mg, 78%) as a white solid. LCMS (ES+) 413 (M+H)⁺.

### Intermediate 20

### (S)-1-[8-Methyl-2-(pyrrolidin-1-yl)quinolin-3-yl]ethanamine

Intermediate 7 (100 mg, 0.31 mmol), pyrrolidine (0.052 mL, 0.62 mmol), DIPEA (0.107 mL, 0.62 mmol) and NMP (2 mL) were combined in a sealed tube and heated under microwave irradiation to 150° C. for 75 minutes. After cooling, conc. HCl (2 mL) was slowly added and the solution was left to stand for 1 h. The reaction mixture was diluted with water (20 mL) and washed with DCM (3×20 mL). The aqueous layer was then basified to pH 10 with 15% NaOH solution and extracted with DCM (3×40 mL). The combined organic layers were dried (MgSO₄) and concentrated in vacuo to give the title compound (79.5 mg, 100%) as a pale yellow gum. LCMS (ES+) 256 (M+H)⁺.

### Intermediate 21

### (S)-tert-Butyl 1-[7-fluoro-8-methyl-2-(3-oxopiperazin-1-yl)quinolin-3-yl]ethylcarbamate

Following the procedure described for Intermediate 5, Intermediate 15 (280 mg, 0.83 mmol), 2-oxopiperazine (86 mg, 1.0 mmol) and DIPEA (0.5 mL, 3.8 mmol) in n-BuOH (2.0 mL) gave the title compound (270 mg, 81%) as a purple oil. δ_{H} (CDCl₃) 7.99 (1H, s), 7.54 (1H, dd, J 8.9, 6.0 Hz), 7.17 (1H, t, J 9.0 Hz), 6.73 (1H, br s), 5.10 (2H, br s), 4.28 (1H, d, J 17.5 Hz), 3.95 (1H, d, J 17.5 Hz), 3.89-3.77 (1H, m), 3.70-3.51 (1H, m), 3.44-3.35 (2H, m), 2.58 (3H, d, J 2.4 Hz), 1.50-1.30 (12H, m).

### Intermediate 22

### (S)-Ethyl 2-(4-{3-[1-(tert-butoxycarbonylamino)ethyl]-8-chloroquinolin-2-yl}piperazin-1-yl)acetate

Following the procedure described for Intermediate 5, Intermediate 4 (150 mg, 0.44 mmol), NMP (2 mL), DIPEA (0.38 mL, 2.2 mmol) and ethyl 2-(piperazin-1-yl)acetate (189 mg, 1.10 mmol) afforded the title compound (116 mg, 55%) as a yellow gum. δ_{H} (CDCl₃) 7.95 (1H, s), 7.68 (1H, dd, J 7.5, 1.3 Hz), 7.59 (1H, dd, J 8.1, 1.4 Hz), 7.27 (1H, t, J 7.84 Hz), 5.11 (1H, br s), 4.96 (1H, br s), 4.22 (2H, dd, J 14.3, 7.1 Hz), 3.67 (2H, d, J 11.3 Hz), 3.39-3.28 (2H, m), 3.32 (2H, s), 2.92-2.85 (2H, m), 2.83-2.74 (2H, m), 1.44 (9H, s), 1.43 (3H, s), 1.30 (3H, t, J 7.1 Hz). LCMS (ES+) 377 (M+H).

### Intermediate 23

### tert-Butyl[8-methyl-2-(3-oxopiperazin-1-yl)quinolin-3-yl]methylcarbamate

tert-Butyl(2-chloro-8-methylquinolin-3-yl)methylcarbamate (500 mg, 1.6 mmol), 2-oxopiperazine (820 mg, 8.2 mmol), NMP (8 mL) and DIPEA (1.4 mL, 8.2 mmol) were combined in a sealed tube and heated to 130° C. for 36 h. After cooling, Et₂O (100 mL) was added to the reaction mixture and the organic layer was washed with water (20 mL) and brine (20 mL). The organic layer was dried (MgSO₄) and filtered, and the solvent was removed in vacuo. Purification by column chromatography (SiO₂, EtOAc) gave the title compound (405 mg, 67%) as a yellow gum. δ_{H} (CDCl₃) 8.00 (1H, s), 7.57 (1H, d, J 8.1 Hz), 7.48 (1H, d, J 7.1 Hz), 7.35-7.24 (1H, m), 6.10 (1H, s), 5.06 (1H, s), 4.51 (2H, d, J 6.1 Hz), 4.16-4.05 (2H, m), 3.50 (4H, s), 2.69 (3H, s), 1.49 (9H, s). LCMS (ES+) 371 (M+H)⁺.

### Intermediate 24

### 4-[3-(Aminomethyl)-8-methylquinolin-2-yl]piperazin-2-one

To a solution of Intermediate 23 (390 mg, 1.1 mmol) in DCM (3 mL) was added TFA (3 mL) and the mixture was stirred for 1 h. The solvents were removed in vacuo and the residue was redissolved in MeOH and passed through a SCX cartridge, eluting with 0-0.1M MeOH/NH₃ in MeOH. The solvent was removed in vacuo to give the title compound (280 mg, 98%). δ_{H} (CDCl₃) 8.09 (1H, s), 7.58 (1H, d, J 8.1 Hz), 7.47 (1H, d, J 7.1 Hz), 7.31 (1H, t, J 7.6 Hz), 5.96 (1H, s), 4.13 (2H, s), 4.05 (2H, s), 3.62-3.49 (4H, m), 2.70 (3H, s), 1.56 (2H, s). LCMS (ES+) 270 (M+H)⁺.

### Intermediate 25

### (S)-1-{4-[3-(1-Aminoethyl)-7-fluoro-8-methylquinolin-2-yl]piperazin-1-yl}-2,2-dimethylpropan-1-one hydrochloride

To a solution of Intermediate 15 (500 mg, 1.48 mmol) in NMP (6 mL) were added 2,2-dimethyl-1-(piperazin-1-yl)propan-1-one (500 mg, 3.00 mmol) and DIPEA (1.3 mL) and the resulting solution was heated at 140° C. for 16 h. The reaction mixture was taken up in EtOAc (150 mL) and washed with water (2×50 mL) and brine (50 mL) The organic phase was separated, dried (phase separation cartridge), and the solvent removed in vacuo. Purification by column chromatography (SiO₂, 10-20% EtOAc in isohexane) gave a beige solid (460 mg, 66%). To a solution of this material (460 mg, 0.97 mmol) in DCM (6 mL) was added TFA (3 mL) and the resulting solution was stirred at r.t. for 15 minutes. The solvents were removed in vacuo. The residue was dissolved in MeOH (6 mL) and placed on an SCX cartridge, washed (MeOH), eluted (7M ammonia in MeOH) and concentrated in vacuo. The residue was redissolved in MeOH (10 mL) and 4M HCl in 1,4-dioxane (5 mL). The solvent was concentrated in vacuo to afford the title compound (397 mg, 100%) as a white solid. δ_{H} (CDCl₃) 8.11 (1H, s), 7.54 (1H, dd, J 8.86, 6.07 Hz), 7.15 (1H, t, J 9.00 Hz), 4.51 (1H, q, J 6.50 Hz), 3.87 (4H, t, J 5.05 Hz), 3.39-3.26 (4H, m), 2.60 (3H, d, J 2.39 Hz), 1.50 (3H, d, J 6.49 Hz), 1.34 (9H, s).

### Intermediate 26

### (S)-1-{4-[3-(1-Aminoethyl)-7-fluoroquinolin-2-yl}piperazin-1-yl]ethanone

To a solution of Intermediate 12 (300 mg, 0.93 mmol) in n-BuOH (3 mL) were added 1-acetylpiperazine (128 mg, 1.0 mmol) and DIPEA (0.3 g, 2.3 mmol) and the resulting solution was heated at 120° C. for 16 h. The reaction mixture was taken up in EtOAc (15 mL) and washed with water (2×5 mL). The organic phase was separated, dried (MgSO₄) and the solvent removed in vacuo. To a solution of the resulting material in DCM (10 mL) was added TFA (3 mL), and the resulting solution was stirred at r.t. for 1 h. The reaction mixture was washed with 2M NaOH solution (2×5 mL) and water (2×5 mL). The organic phase was separated, dried (MgSO₄) and the solvent removed in vacuo to afford the title compound (280 mg, 86%) as a yellow gum. δ_{H} (CDCl₃) 8.21 (1H, s), 7.68 (1H, dd, J 7.2 Hz), 7.49 (1H, d, J 7.2 Hz), 7.19 (1H, t, J 8.4 Hz), 4.90 (2H, br s), 4.62 (1H, br q, J 6.8 Hz), 3.85 (2H, br s), 3.68 (2H, br s), 3.38 (2H, br s), 3.25 (5H, br s), 1.61 (3H, d, J 6.8 Hz).

### Intermediate 27

### (S)-tert-Butyl 1-[7-fluoro-8-methyl-2-(pyrrolidin-1-yl)quinolin-3-yl]ethylcarbamate

To a solution of Intermediate 15 (280 mg, 0.83 mmol) in n-BuOH (3 mL) were added pyrrolidine (0.20 mL, 2.33 mmol) and DIPEA (0.42 mL, 2.33 mmol). The resulting solution was heated at 120° C. for 16 h. The mixture was diluted with EtOAc (50 mL) and Et₂O (50 mL) and washed with brine (3×25 mL). The organic layer was separated, dried (MgSO₄), filtered and the solvent removed in vacuo. Purification by column chromatography (SiO₂, 5-10% Et₂O in petrol 40-60) gave the title compound (270 mg, 87%) as a pale yellow oil. LCMS (ES+) 375 (M+H)⁺.

### Intermediate 28

### (S)-1-{4-[3-(1-Aminoethyl)-7-fluoro-8-methylquinolin-2-yl]piperazin-1-yl}ethanone

To a solution of Intermediate 15 (280 mg, 0.83 mmol) in n-BuOH (3 mL) were added 1-acetylpiperazine (128 mg, 1.0 mmol) and DIPEA (0.3 g, 2.3 mmol) and the resulting solution was heated at 120° C. for 16 h. The reaction mixture was taken up in EtOAc (15 mL) and washed with water (2×5 mL). The organic phase was separated, dried (MgSO₄) and the solvent removed in vacuo. To a solution of the resulting material in DCM (10 mL) was added TFA (3 mL), and the resulting solution was stirred at r.t. for 1 h. The reaction mixture was washed with 2M NaOH solution (2×5 mL) and water (2×5 mL). The organic phase was separated, dried (MgSO₄) and the solvent removed in vacuo to afford the title compound (140 mg, 52%) as a orange oil. δ_{H} (CDCl₃) 7.66 (1H, s), 7.38 (1H, dd, J 6.4 Hz), 6.93 (1H, t, J 8.8 Hz), 4.90 (2H, br s), 4.55 (1H, br q, J 6.4 Hz), 3.83-3.90 (2H, m), 3.75-3.83 (2H, m), 3.64-3.72 (4H, m), 3.42 (3H, s) 2.50 (3H, s), 1.61 (3H, d, J 6.8 Hz).

### Intermediate 29

### (S)-4-[3(1-Aminoethyl)-7-fluoroquinolin-2-yl]piperazin-2-one

To a solution of Intermediate 12 (300 mg, 0.93 mmol) in n-BuOH (3 mL) were added 2-oxopiperazine (100 mg, 1.0 mmol) and DIPEA (0.3 g, 2.3 mmol) and the resulting solution was heated at 120° C. for 16 h. The reaction mixture was taken up in EtOAc (15 mL) and washed with water (2×5 mL). The organic phase was separated, dried (MgSO₄) and the solvent removed in vacuo. To a solution of the resulting material in DCM (10 mL) was added TFA (3 mL), and the resulting solution was stirred at r.t. for 1 h. The reaction mixture was washed with 2M NaOH solution (2×5 mL) and water (2×5 mL). The organic phase was separated, dried (MgSO₄) and the solvent removed in vacuo to afford the title compound (146 mg, 51%) as a yellow gum. δ_{H} (CDCl₃) 8.21 (1H, s), 7.68 (1H, dd, J 7.2 Hz), 7.49 (1H, d, J 7.2 Hz), 7.19 (1H, t, J 8.4 Hz), 6.55 (1H, br s), 5.20 (2H, br s), 5.10 (1H, q, J 4.8 Hz), 4.70 (1H, d, J 17.2 Hz,), 3.95-4.20 (3H, m), 2.85-3.08 (2H, m), 1.50 (3H, d, J 7.6 Hz,).

### Intermediate 30

### (S)-4-[3-(1-Aminoethyl)-5,7-difluoroquinolin-2-yl]piperazin-2-one

To a solution of Intermediate 18 (600 mg, 1.75 mmol) in n-BuOH (6 mL) were added 2-oxopiperazine (200 mg, 2.0 mmol) and DIPEA (0.3 g, 2.3 mmol) and the resulting solution was heated at 120° C. for 48 h. The reaction mixture was taken up in EtOAc (15 mL) and washed with water (2×5 mL). The organic phase was separated, dried (MgSO₄) and the solvent removed in vacuo. To a solution of the resulting material in DCM (10 mL) was added TFA (3 mL), and the resulting solution was stirred at r.t. for 1 h. The reaction mixture was washed with 2M NaOH solution (2×5 mL) and water (2×5 mL). The organic phase was separated, dried (MgSO₄) and the solvent removed in vacuo to afford the title compound (180 mg, 34%) as a yellow gum. δ_{H} (CDCl₃) 8.42 (1H, s), 7.40 (1H, d, J 9.6 Hz), 6.95 (1H, t, J 7.2 Hz), 6.60 (1H, s), 5.40 (2H, br s), 5.15 (1H, q, J 6.8 Hz), 4.15 (1H, d, J 17.2 Hz), 4.05 (1H, d, J 17.2 Hz), 3.10-3.28 (2H, m), 2.83-3.08 (2H, m), 1.50 (3H, d, J 7.6 Hz).

### Intermediate 31

### (S)-tert-Butyl 1-[2-(4-acetylpiperazin-1-yl)-8-chloroquinolin-3-yl]ethylcarbamate

Following the procedure described for Intermediate 5, Intermediate 4 (300 mg, 0.88 mmol), 1-(piperazin-1-yl)ethanone (564 mg, 4.40 mmol), NMP (3 mL) and DIPEA (1.53 mL, 8.80 mmol) gave the title compound (0.25 mg, 65%) as a clear glass. δ_{H} (CDCl₃) 8.01 (1H, s), 7.71 (1H, dd, J 7.5, 1.4 Hz), 7.62 (1H, dd, J 8.0, 1.4 Hz), 7.31 (1H, t, J 7.8 Hz), 5.08 (1H, m), 3.94-3.71 (3H, m), 3.67-3.52 (3H, m), 3.39-3.15 (2H, m), 2.16 (3H, s), 1.76 (1H, d, J 2.5 Hz), 1.48-1.43 (12H, m).

### Intermediate 32

### (S)-1-{4-[3-(1-Aminoethyl)-5,7-difluoroquinolin-2-yl]piperazin-1-yl}ethanone

To a solution of Intermediate 18 (500 mg, 1.46 mmol) in n-BuOH (5 mL) were added 1-acetylpiperazine (200 mg, 1.6 mmol) and DIPEA (0.36 g, 2.8 mmol) and the resulting solution was heated at 100° C. for 72 h. The reaction mixture was taken up in EtOAc (15 mL) and washed with water (2×5 mL). The organic phase was separated, dried (MgSO₄) and the solvent removed in vacuo. To a solution of the resulting material in DCM (10 mL) was added TFA (3 mL), and the resulting solution was stirred at r.t. for 1 h. The reaction mixture was washed with 2M NaOH solution (2×5 mL) and water (2×5 mL). The organic phase was separated, dried (MgSO₄) and the solvent removed in vacuo to afford the title compound (430 mg, 88%) as an orange oil. δ_{H} (CDCl₃) 8.35 (1H, s), 7.32 (1H, d, J 8.8 Hz), 6.88 (1H, t, J 8.8 Hz), 4.70 (2H, br s), 4.45 (1H, q, J 6.4 Hz), 3.78-3.82 (2H, m), 3.64-3.68 (2H, m), 3.20-3.42 (4H, br m), 2.20 (3H, s), 1.49 (3H, d, J 6.8 Hz).

### Intermediate 33

### tert-Butyl(2-chloro-7-fluoro-8-methylquinolin-3-yl)methylcarbamate

To a solution of Intermediate 13 (2.0 g, 6.14 mmol) in EtOH (20 mL) was added NaBH₄ (400 mg, 10.52 mmol). The reaction mixture was stirred at r.t. for 4 h, quenched with 2M HCl solution (50 mL), stirred for 20 minutes, neutralised with 2M NaOH solution and extracted with DCM (100 mL). The organic phase was separated, dried (MgSO₄) and filtered. To this filtrate was added DIPEA (2.0 mL, 10.0 mmol), followed by a solution of di-tert-butyl dicarbonate (2.0 g, 9.16 mmol) in DCM (10 mL) dropwise. The reaction mixture was stirred at r.t. for 3 h, then diluted with DCM (10 mL) and washed with saturated NaHCO₃ solution (15 mL) and brine (15 mL). The organic layer was dried (MgSO₄), concentrated in vacuo and purified by column chromatography (SiO₂, 0-30% EtOAc in 40-60 petroleum ether), to give the title compound (1.6 g, 81%) as a pale yellow solid. δ_{H} (CDCl₃) 8.11 (1H, s), 7.65 (1H, d, J 7.2 Hz), 7.30 (1H, t, J 8.4 Hz), 5.20 (1H, br s), 4.54 (2H, br s), 2.65 (3H, s), 1.48 (9H, s).

### Intermediate 34

### 1-{4-[3-(Aminomethyl)-7-fluoro-8-methylquinolin-2-yl]piperazin-1-yl}ethanone

To a solution of Intermediate 33 (500 mg, 1.55 mmol) in n-BuOH (5 mL) were added 1-acetylpiperazine (200 mg, 1.6 mmol) and DIPEA (0.36 g, 2.8 mmol) and the resulting solution was heated at 100° C. for 72 h. The reaction mixture was taken up in EtOAc (15 mL) and washed with water (2×5 mL). The organic phase was separated, dried (MgSO₄) and the solvent removed in vacuo. To a solution of the resulting material in DCM (5 mL) was added TFA (1 mL), and the resulting solution was stirred at r.t. for 1 h. The reaction mixture was washed with 2M NaOH solution (2×5 mL) and water (2×5 mL). The organic phase was separated, dried (MgSO₄) and the solvent removed in vacuo to afford the title compound (150 mg, 31%) as a white solid. δ_{H} (CDCl₃) 8.05 (1H, s), 7.53 (1H, dd, J 8.8 Hz), 7.12 (1H, t, J 8.8 Hz), 4.05 (2H, s), 3.80-3.83 (2H, m), 3.64-3.68 (1H, m), 3.38-3.42 (2H, m), 3.22-3.25 (3H, m), 2.60 (3H, s), 2.15 (3H, s), 2.00 (2H, br s).

### Intermediate 35

### (S)-1-{4-[3-(1-Aminoethyl)quinolin-2-yl]piperazin-1-yl}ethanone

To a solution of Intermediate 31 (500 mg, 1.15 mmol) in EtOH (10 mL) were added hydrazine hydrate (200 mg, 4.0 mmol) and 10% palladium on carbon (0.05 g). The reaction mixture was heated at 80° C. for 72 h. The mixture was filtered through Celite and the filtrate was concentrated in vacuo. The residue obtained was purified by column chromatography (SiO₂, 3% MeOH in DCM) to give an orange oil (300 mg, 66%). To a solution of this material in DCM (5 mL) was added TFA (1 mL), and the resulting solution was stirred at r.t. for 1 h. The reaction mixture was washed with 2M NaOH solution (2×5 mL) and water (2×5 mL). The organic phase was separated, dried (MgSO₄) and the solvent was removed in vacuo to afford the title compound (210 mg, 91%) as a yellow oil. δ_{H} (CDCl₃) 8.10 (1H, s), 7.89 (1H, d, J 8.8 Hz), 7.72 (1H, d, J 8.8 Hz), 7.66 (1H, t, J 8.8 Hz), 7.63 (1H, t, J 8.8 Hz), 5.17-5.23 (1H, m), 4.75 (2H, br s), 3.92-3.95 (1H, m), 3.79-3.83 (2H, m), 3.64-3.68 (1H, m), 3.35-3.38 (2H, m), 3.18-3.22 (2H, m), 2.22 (3H, s), 1.84 (3H, d, J 6.8 Hz).

### Intermediate 36

### (S)-4-[3-(1-Aminoethyl)-7-fluoro-8-methylquinolin-2-yl]piperazine-1-carboxamide

A solution of Intermediate 15 (2 g, 5.9 mmol), piperazine (2.6 g, 30.2 mmol) and DIPEA (5.1 mL, 29.4 mmol) in NMP (12 mL) was heated at 140° C. for 16 h. After cooling, the mixture was dissolved in EtOAc (150 mL) and washed with water (2×50 mL) and saturated brine (50 mL). The organic layer was dried using a phase separation cartridge and concentrated in vacuo to give a white foam (2.29 g, 100%). LCMS (ES+) 389 (M+H)⁺. To a solution of this foam (400 mg, 1.03 mmol) and Et₃N (0.43 mL, 3.09 mmol) in DCM (5 mL) was added trimethylsilyl isocyanate (154 mg, 1.34 mmol). The reaction mixture was stirred at r.t. for 17 h and the excess solvent was removed in vacuo. Purification by column chromatography (SiO₂, 95:4:1 DCM/MeOH/NH₃ solution in MeOH) gave a colourless glass (419 mg, 94%). LCMS (ES+) 432 (M+H)⁺. To this glass (402 mg, 0.932 mmol) dissolved in DCM (24 mL) was added TFA (4.2 mL). The reaction mixture was stirred at r.t. for 1.5 h. The excess solvent was removed in vacuo. The oil obtained was basified with 0.2M NaOH solution (40 mL) and extracted with EtOAc (3×80 mL). The combined organic layers were dried (MgSO₄), filtered and concentrated in vacuo to give the title compound (349 mg, 100%) as a white foam. δ_{H} (DMSO-d₆) 8.39 (s, 1H), 7.75 (dd, J 8.92, 6.37 Hz, 1H), 7.31 (t, J 9.14 Hz, 1H), 6.08 (s, 2H), 4.39-4.34 (m, 1H), 3.46-3.59 (m, 5H), 3.35 (m, 2H, masked by water peak), 3.12-3.19 (m, 3H), 2.55 (d, J 2.40 Hz, 3H), 1.39 (d, J 6.42 Hz, 3H). LCMS (ES+) 332 (M+H)⁺.

### Intermediate 37

### (S)-1-[4-(8-Methyl-3-{1-[2-(methylsulfonyl)pyrazolo[1,5-a][1,3,5]triazin-4-ylamino]ethyl}quinolin-2-yl)piperazin-1-yl]ethanone

To a solution of Example 4 (160 mg, 0.36 mmol) in DCM (8 mL) was added 3-chloroperoxybenzoic acid (217 mg, 1.26 mmol). After 2.5 h, the mixture was washed with water (2×10 mL). The organic layer was separated, dried (MgSO₄) and the solvent was removed in vacuo. Purification by preparative HPLC gave the title compound (55 mg, 17%) as a white solid. δ_{H} (DMSO-d₆) 8.45 (1H, s), 8.41 (1H, d, J 2.18 Hz), 7.67-7.60 (1H, m), 7.55-7.49 (1H, m), 7.38-7.29 (1H, m), 6.80 (1H, d, J 2.18 Hz), 5.94-5.85 (1H, m), 3.83-3.56 (6H, m), 3.19-3.01 (3H, m), 2.64 (3H, s), 2.09 (3H, s), 2.07 (3H, s), 1.68 (3H, d, J 6.74 Hz).

### Intermediate 38

### 4-(8-Methyl-3-{[2-(methylthio)pyrazolo[1,5-a][1,3,5]triazin-4-ylamino]methyl}quinolin-2-yl)piperazin-2-one

Intermediate 24 (0.28 g, 1.04 mmol) in THF (5 mL) was treated with DIPEA (0.54 mL, 3.1 mmol) and 4-chloro-2-(methylthio)pyrazolo[1,5-a][1,3,5]triazine (233 mg, 1.14 mmol). The reaction mixture was stirred for 16 h, then partitioned between water (10 mL) and DCM (20 mL). The organic layer was dried (phase separation cartridge) and concentrated in vacuo. The residue was purified by column chromatography (SiO₂, 0-100% EtOAc in isohexane) to give the title compound (178 mg, 38%) as a white solid. δ_{H} (DMSO-d₆) 9.55 (1H, br s), 8.17-8.12 (2H, m), 7.96 (1H, s), 7.68 (1H, d, J 8.09 Hz), 7.53 (1H, d, J 7.03 Hz), 7.31 (1H, t, J 7.57 Hz), 6.34 (1H, d, J 2.11 Hz), 4.88 (2H, s), 3.95 (2H, s), 3.54 (2H, t, J 5.16 Hz), 3.36 (2H, s), 2.66 (3H, s), 2.41 (3H, s). LCMS (ES+) 435 (M+H)⁺.

### Intermediate 39

### N—[(R)-1-(2,8-Dichloroquinolin-3-yl)ethyl]-(R)=2-methylpropane-2-sulfinamide

Purification by column chromatography (SiO₂, 0-50% EtOAc in isohexane) of the ether filtrate from Intermediate 2 afforded the title compound (1.9 g, 14%) as a colourless solid. δ_{H} (CDCl₃) 8.28 (1H, s), 7.86-7.73 (2H, m), 7.53-7.45 (1H, m), 5.10-5.01 (1H, m), 3.81 (1H, d, J 5.32 Hz), 1.66 (3H, d, J 6.66 Hz), 1.26 (9H, s).

### Intermediate 40

### (R)-tert-Butyl 1-(2,8-dichloroquinolin-3-yl)ethylcarbamate

4N HCl in 1,4-dioxane (2.9 mL, 11.6 mmol) was added to a stirred solution of Intermediate 39 (1.9 g, 5.5 mmol) in MeOH (12.6 mL) and the reaction mixture was stirred at r.t. for 2 h. The mixture was concentrated under reduced pressure to give a pale yellow gum (1.8 g). To this gum dissolved in DCM (85 mL) were added di-tert-butyl Bicarbonate (2.55 g, 11.7 mmol) and DIPEA (5.1 mL, 29.1 mmol). The resulting mixture was stirred at r.t. for 24 h. The mixture was washed with saturated aqueous NaHCO₃ (20 mL), and the organic layer was separated, dried (MgSO₄) and the solvent was removed in vacuo. Crystallisation from 40-60 petroleum ether afforded the title compound (1.1 g, 59%) as a white solid. δ_{H} (CDCl₃) 8.13 (1H, s), 7.81 (1H, d, J 7.51 Hz), 7.73 (1H, dd, J 8.17, 1.32 Hz), 7.51-7.42 (1H, m), 5.28-5.04 (2H, m), 1.72-1.28 (12H, m).

### Intermediate 41

### (R)-tert-Butyl 1-[8-chloro-2-(3-oxopiperazin-1-yl)quinolin-3-yl]ethylcarbamate

Following the procedure described for Intermediate 5, Intermediate 40 (1.1 g, 3.43 mmol) and 2-oxopiperazine (1.73 g, 17.3 mmol) gave the title compound (850 mg, 59%) as a white solid. δ_{H} (CDCl₃) 8.03 (1H, s), 7.72 (1H, dd, J 7.52, 1.32 Hz), 7.63 (1H, dd, J 8.09, 1.32 Hz), 7.32 (1H, t, J 7.81 Hz), 6.11 (1H, s), 5.19-4.88 (2H, m), 4.43-4.29 (1H, m), 4.10-3.87 (2H, m), 3.79-3.69 (1H, m), 3.56-3.35 (2H, m), 1.47-1.38 (12H, m).

### Intermediate 42

### (S)—N-[1-(2-Chloro-7-fluoro-8-methylquinolin-3-yl)ethyl]pyrazolo[1,5-a]pyrimidin-7-amine

Intermediate 15 (0.52 g, 1.54 mmol) and 4N HCl in 1,4-dioxane (10 mL) were stirred at r.t. for 40 minutes. The excess solvent was removed in vacuo and the residue obtained was partitioned between DCM (50 mL) and saturated NaHCO₃ solution (10 mL). The organic layer was dried (MgSO₄) and concentrated in vacuo. The residue (0.35 g, 1.47 mmol) was dissolved in n-BuOH (10 mL) and 7-chloropyrazolo[1,5-a]pyrimidine (0.23 g, 1.49 mmol) was added. The mixture was heated at 100° C. for 3 h. The solvent was removed in vacuo and the residue purified by column chromatography (SiO₂, 50% EtOAc in 40-60 petroleum ether) to afford the title compound (440 mg, 80%) as a pale yellow foam. δ_{H} (CDCl₃) 8.10 (m, 3H), 7.56 (dd, J 9.1, 6.1 Hz, 1H), 7.29 (m, 1H), 6.82 (d, J 6.1 Hz, 1H), 6.56 (d, J 2.3 Hz, 1H), 5.65 (d, J 5.1 Hz, 1H), 5.29 (m, 1H), 2.67 (d, J 2.3 Hz, 3H), 1.82 (d, J 6.8 Hz, 3H). LCMS (ES+) 356 (M+H)⁺.

### Example 1

### (S)-4-(8-Chloro-3-{1-[2-(methylthio)pyrazolo[1,5-a][1,3,5]triazin-4-ylamino]ethyl}-quinolin-2-yl)piperazin-2-one

To a solution of Intermediate 5 (1.0 g, 3.17 mmol) in 1,4-dioxane (15 mL) at r.t. was added HCl (15.8 mL, 63.4 mmol; 4M in 1,4-dioxane). After 2 h, the solvent was removed in vacuo. The residue was redissolved in anhydrous THF (6 mL). DIPEA (1.66 mL, 9.51 mmol) was added, followed by 4-chloro-2-(methylthio)pyrazolo[1,5-a][1,3,5]triazine (950 mg, 4.75 mmol). The reaction mixture was stirred at r.t. for 2 h. The solvent was removed in vacuo. The residue was redissolved in DCM and was washed with water (2×15 mL). The organic layer was dried (MgSO₄), and the solvent removed in vacuo. Following purification by preparative HPLC the title compound (276 mg, 19%) was obtained as a white solid. δ_{H} (CDCl₃) 8.11 (s, 1H), 7.93 (d, J 2.1 Hz, 1H), 7.74 (dd, J 7.5, 1.3 Hz, 1H), 7.62 (dd, J 8.1, 1.3 Hz, 1H), 7.30-7.35 (m, 1H), 6.94 (d, J 7.4 Hz, 1H), 6.28 (d, J 2.1 Hz, 1H), 6.01 (s, 1H), 5.68-5.74 (m, 1H), 4.40 (d, J 17.5 Hz, 1H), 4.00-4.14 (m, 2H), 3.73-3.83 (m, 1H), 3.55-3.63 (m, 2H), 2.49 (s, 3H), 1.67 (d, J 6.8 Hz, 3H). LCMS (ES+) 469/471 (M+H)⁺, RT 3.13 minutes (Method 1).

### Example 2

### (S)-4-(8-Chloro-3-{1-[2-(methylsulfonyl)pyrazolo[1,5-a][1,3,5]triazin-4-ylamino]ethyl}-quinolin-2-yl)piperazin-2-one

To a solution of Example 1 (150 mg, 0.32 mmol) in DCM (3 mL) at r.t. was added dry 3-chloroperoxybenzoic acid (136 mg, 0.8 mmol). After 2 h, the mixture was washed twice with saturated aqueous NaHCO₃. The organic layer was separated, dried (MgSO₄) and the solvent was removed in vacuo. Purification by column chromatography (SiO₂, 0-5% MeOH in DCM) gave the title compound which, after freeze-drying, was obtained as a white solid (40 mg, 25%). δ_{H} (CDCl₃) 8.17 (d, J 2.2 Hz, 1H), 8.12 (s, 1H), 7.75 (dd, J 7.5, 1.3 Hz, 1H), 7.62 (dd, J 8.1, 1.3 Hz, 1H), 7.54 (d, J 8.0 Hz, 1H), 7.30-7.36 (m, 1H), 6.69 (d, J 2.2 Hz, 1H), 6.00 (s, 1H), 5.89-5.95 (m, 1H), 4.35 (d, J 17.4 Hz, 1H), 4.11-4.18 (m, 1H), 4.01 (d, J 17.7 Hz, 1H), 3.68-3.75 (m, 1H), 3.60-3.68 (m, 2H), 3.26 (s, 3H), 1.74 (d, J 6.8 Hz, 3H).

### Example 3

### (S)-4-{8-Chloro-3-[1-(pyrazolo[1,5-a][1,3,5]triazin-4-ylamino)ethyl]quinolin-2-yl}piperazin-2-one

To a solution of Example 2 (31 mg, 0.066 mmol) in a 1:1 mixture of EtOH/CHCl₃ (0.7 mL) at r.t. was added NaBH₄ (4.7 mg, 0.12 mmol) portionwise. After 15 minutes, water (2 mL) was added to the mixture. The solvents were removed in vacuo and the crude was redissolved in DCM and washed with water (2×3 mL). The organic layer was separated, dried (MgSO₄), filtered and the solvent was removed in vacuo. Purification by column chromatography (SiO₂, 0-5% MeOH in DCM) gave the title compound which, after freeze-drying, was obtained as a white solid (12.5 mg, 48%). δ_{H} (DMSO-d₆) 9.50 (s, 1H), 8.57 (s, 1H), 8.25 (d, J 2.2 Hz, 1H), 8.13 (s, 1H), 8.00 (s, 1H), 7.82-7.87 (m, 2H), 7.44 (dd, J 8.1, 7.5 Hz, 1H), 6.54 (d, J 2.1 Hz, 1H), 5.79 (q, J 6.8 Hz, 1H), 4.14 (d, J 17.1 Hz, 1H), 3.85-4.07 (m, 2H), 3.53-3.60 (m, 1H), 3.30-3.48 (m, 2H, one H masked by solvent peak), 1.66 (d, J 6.7 Hz, 3H). LCMS (ES+) 423/425 (M+H)⁺, RT 2.87 minutes (Method 2).

### Example 4

### (S)-1-[4-(8-Methyl-3-{1-[2-(methylthio)pyrazolo[1,5-a][1,3,5]triazin-4-ylamino]ethyl}-quinolin-2-yl)piperazin-1-yl]ethanone

To a solution of Intermediate 19 (950 mg, 2.3 mmol) in DCM (10 mL) was added TFA (3 mL) and the resulting solution was stirred at r.t. for 2 h. The solvents were removed in vacuo. The residue was redissolved in MeOH (6 mL) and loaded on an SCX cartridge, washed (MeOH), eluted (7M ammonia in MeOH) and concentrated in vacuo. The residue was redissolved in anhydrous THF (15 mL). DIPEA (1.0 mL, 5.76 mmol) was added, followed by 4-chloro-2-(methylthio)pyrazolo[1,5-a][1,3,5]triazine (553 mg, 2.76 mmol). The reaction mixture was stirred for 2 h. The solvent was removed in vacuo. The residue was redissolved in DCM and was washed with water (2×15 mL). The organic layer was dried (MgSO₄) and the solvent removed in vacuo. Purification by column chromatography (SiO₂, 0-80% EtOAc in isohexane) gave the title compound (817 mg, 75%) which, after freeze-drying, was obtained as a white solid. δ_{H} (CDCl₃) 8.07 (1H, s), 7.91 (1H, d, J 2.12 Hz), 7.56 (1H, d, J 8.07 Hz), 7.49 (1H, d, J 6.99 Hz), 7.36-7.23 (2H, m), 6.26 (1H, d, J 2.11 Hz), 5.85-5.75 (1H, m), 4.00-3.72 (4H, m), 3.64-3.50 (2H, m), 3.24-3.13 (2H, m), 2.70 (3H, s), 2.52 (3H, s), 2.19 (3H, s), 1.69 (3H, d, J 6.80 Hz). LCMS (ES+) 477 (M+H)⁺, RT 3.99 minutes (Method 2).

### Example 5

### 1-[4-(8-Methyl-3-{(1S)-1-[2-(methylsulfinyl)pyrazolo[1,5-a][1,3,5]triazin-4-ylamino]-ethyl}quinolin-2-yl)piperazin-1-yl]ethanone

To a solution of Example 4 (160 mg, 0.36 mmol) in DCM (8 mL) was added 3-chloroperoxybenzoic acid (217 mg, 1.26 mmol). After 2.5 h, the mixture was washed with water (2×5 mL). The organic layer was separated, dried (MgSO₄) and the solvent was removed in vacuo. Purification by preparative HPLC gave the title compound (65 mg, 21%) as a white solid, in the form of a mixture of two diastereoisomeric sulfoxides. δ_{H} (DMSO-d₆) 10.03 (1H, s), 8.50 and 8.45 (1H, 2s), 8.35 (1H, t, J 2.38 Hz), 7.69-7.63 (1H, m), 7.55-7.50 (1H, m), 7.40-7.32 (1H, m), 6.68 (1H, d, J 2.17 Hz), 5.93-5.82 (1H, m), 3.80-3.45 (6H, m), 3.24-3.02 (2H, m), 2.84 and 2.74 (3H, 2s), 2.66 (3H, s), 2.11 (3H, s), 1.73-1.67 (3H, m). LCMS (ES+) 493 (M+H)⁺, RT 3.02 minutes (Method 2).

### Example 6

### (S)-1-(4-{8-Methyl-3-[1-(pyrazolo[1,5-a][1,3,5]triazin-4-ylamino)ethyl]quinolin-2-yl}piperazin-1-yl)ethanone

To a solution of Intermediate 37 (55 mg, 0.066 mmol) in a 1:1 mixture of EtOH/CHCl₃ (0.7 mL) was added NaBH₄ (8.2 mg, 0.22 mmol) portionwise. After 1 h, water (2 mL) was added to the mixture. The solvents were removed in vacuo and the crude residue was redissolved in DCM (10 mL) and washed with water (2×3 mL). The organic layer was separated, dried (MgSO₄) and filtered, and the solvent was removed in vacuo. Purification by column chromatography (SiO₂, 0-50% EtOAc in isohexane) gave the title compound (32 mg, 68%) which, after freeze-drying, was obtained as a white solid. δ_{H} (DMSO-d₆) 9.44 (1H, s), 8.46 (1H, s), 8.22 (1H, d, J 2.14 Hz), 8.17 (1H, s), 7.69-7.63 (1H, m), 7.55-7.49 (1H, m), 7.38-7.30 (1H, m), 6.51 (1H, d, J 2.15 Hz), 5.93-5.83 (1H, m), 3.76-3.61 (4H, m), 3.59-3.44 (2H, m), 3.15-3.01 (2H, m), 2.64 (3H, s), 2.08 (3H, s), 1.65 (3H, d, J 6.77 Hz). LCMS (ES+) 431 (M+H)⁺, RT 2.85 minutes (Method 1).

### Example 7

### (S)-4-{8-Chloro-3-[1-(pyrazolo[1,5-a]pyrimidin-7-ylamino)ethyl]quinolin-2-yl}-piperazin-2-one

To a solution of Intermediate 5 (1.0 g, 3.17 mmol) in 1,4-dioxane (15 mL) was added HCl (15.8 mL, 63.4 mmol; 4M in 1,4-dioxane). After 2 h, the solvent was removed in vacuo. The residue (100 mg, 0.33 mmol), NMP (2 mL), DIPEA (0.17 mL, 0.98 mmol) and 7-chloropyrazolo[1,5-a]pyrimidine (76 mg, 0.5 mmol) were combined in a sealed tube and heated to 140° C. overnight. The reaction mixture was cooled and the solvent removed in vacuo. The residue was redissolved in DCM (20 mL) and washed with water (10 mL) and brine (10 mL). The organic layer was separated, dried (MgSO₄) and filtered, and the solvent was removed in vacuo. Purification by preparative HPLC gave the title compound (16.7 mg, 12%) as a light orange solid. δ_{H} (DMSO-d₆) 8.67 (1H, s), 8.12-8.07 (2H, m), 8.04 (1H, s), 7.85-7.79 (2H, m), 7.42-7.37 (1H, m), 6.43 (1H, d, J 2.28 Hz), 6.10 (1H, d, J 5.28 Hz), 5.14-5.07 (1H, m), 4.12 (1H, d, J 17.14 Hz), 3.90 (1H, d, J 17.13 Hz), 3.63-3.45 (4H, m), 1.87 (3H, d, J 6.67 Hz). NH not seen. LCMS (ES+) 422/424 (M+H)⁺, RT 2.13 minutes (Method 2).

### Example 8

### (S)—N-{1-[8-Methyl-2-(pyrrolidin-1-yl)quinolin-3-yl]ethyl}pyrazolo[1,5-a][1,3,5]triazin-4-amine

Pyrazolo[1,5-a][1,3,5]triazin-4(1H)-one (900 mg, 6.61 mmol) was suspended in POCl₃ (11.5 mL) in the presence of DMAP (2.83 g, 23.1 mmol). The resulting mixture was heated under reflux for 2 h. After cooling, the excess solvent was concentrated in vacuo. A portion of the residue (45 mg, 0.29 mmol) was combined with Intermediate 20 (50 mg, 0.2 mmol) and DIPEA (0.35 mL, 1.96 mmol) in dry THF (3 mL) and stirred for 2 h. The mixture was diluted with DCM (20 mL) and washed with water (10 mL). The organic layer was separated, dried (MgSO₄) and filtered, and the solvent was removed in vacuo. Purification by preparative HPLC gave the title compound (24.9 mg, 33%) as a white solid. δ_{H} (CDCl₃) 8.23 (1H, s), 8.03 (1H, d, J 2.16 Hz), 8.01 (1H, s), 7.52-7.45 (1H, m), 7.47-7.41 (1H, m), 7.21-7.13 (1H, m), 7.03-6.96 (1H, m), 6.50 (1H, d, J 2.17 Hz), 6.03-5.93 (1H, m), 3.87-3.78 (2H, m), 3.77-3.69 (2H, m), 2.68 (3H, s), 2.10-1.93 (4H, m), 1.73 (3H, d, J 6.73 Hz). LCMS (ES+) 374 (M+H)⁺, RT 2.38 minutes (Method 2).

### Example 9

### (S)-4-{3-[1-(2-Aminopyrazolo[1,5-a][1,3,5]triazin-4-ylamino)ethyl]-8-chloroquinolin-2-yl}piperazin-2-one

To a solution of Example 2 (84 mg, 0.17 mmol) in 1,4-dioxane (1.5 mL) was added ammonium hydroxide (13 μL, 0.34 mmol). The mixture was heated at 70° C. overnight. The solvent was removed in vacuo. The mixture was washed with saturated aqueous NaHCO₃ solution (2×3 mL). The organic layer was separated, dried (MgSO₄) and the solvent was removed in vacuo. Purification by column chromatography (SiO₂, 0-5% MeOH in DCM) gave the title compound (69 mg, 93%) as a white solid. δ_{H} (CDCl₃) 9.06 (1H, d, J 7.67 Hz), 8.52 (1H, s), 8.14 (1H, s), 7.91 (1H, d, J 2.04 Hz), 7.85-7.78 (2H, m), 7.43-7.38 (1H, m), 6.63 (2H, s), 5.81 (1H, d, J 2.04 Hz), 5.60-5.54 (1H, m), 4.64 (1H, d, J 17.20 Hz), 4.50 (1H, d, J 17.18 Hz), 4.02-3.92 (2H, m), 3.54-3.30 (2H, m), 1.52 (3H, d, J 6.65 Hz). LCMS (ES+) 438/440 (M+H)⁺, RT 2.31 minutes (Method 2).

### Example 10

### (S)-4-(8-Chloro-3-{1-[2-(methylamino)pyrazolo[1,5-a][1,3,5]triazin-4-ylamino]ethyl}quinolin-2-yl)piperazin-2-one

To a solution of Example 2 (84 mg, 0.17 mmol) in 1,4-dioxane (1.5 mL) was added methylamine (0.17 mL, 0.34 mmol; 2M in THF). The reaction mixture was heated at 50° C. for 2 h. After cooling, the solvent was removed in vacuo. The residue obtained was extracted with EtOAc (20 mL) and washed with saturated aqueous NaHCO₃ solution (2×10 mL). The organic layer was separated, dried (MgSO₄) and the solvent was removed in vacuo. Purification by column chromatography (SiO₂, 0-5% MeOH in DCM) gave the title compound (69 mg, 91%) which, after freeze-drying, was obtained as a white solid. δ_{H} (CDCl₃) 8.10 (1H, s), 7.80 (1H, d, J 2.10 Hz), 7.69 (1H, dd, J 7.52, 1.29 Hz), 7.58 (1H, dd, J 8.07, 1.30 Hz), 6.79 (2H, d, J 6.81 Hz), 6.04-6.00 (1H, m), 5.99-5.97 (1H, m), 5.56-5.41 (2H, m), 4.58-4.43 (2H, m), 4.27-4.18 (2H, m), 3.63-3.48 (2H, m), 2.91 (3H, d, J 4.87 Hz), 1.51 (3H, d, J 6.65 Hz). LCMS (ES+) 452/454 (M+H)⁺, RT 2.36 minutes (Method 2).

### Example 11

### (S)-4-{7-Fluoro-8-methyl-3-[1-(pyrazolo[1,5-a][1,3,5]triazin-4-ylamino)ethyl]quinolin-2-yl}piperazin-2-one

TFA (2 mL) was added to a stirred solution of Intermediate 21 (161 mg, 0.40 mmol) in DCM (5 mL) and the mixture was allowed to stand at r.t. for 3 h before being concentrated in vacuo. The residue was dissolved in DCM (20 mL) and washed with 2M NaOH (2×10 mL) and water (10 mL). The organic layer was separated, dried (MgSO₄), filtered and concentrated in vacuo. To the residue (160 mg, 0.40 mmol) and DIPEA (0.28 mL, 1.62 mmol) dissolved in n-BuOH (3.3 mL) was added 4-chloro-2-(methylthio)-pyrazolo[1,5-a][1,3,5]triazine (162 mg, 0.81 mmol) and the reaction mixture was stirred at r.t. for 2 h. The solvent was removed in vacuo. The residue was redissolved in DCM (20 mL) and washed with water (2×15 mL). The organic layer was separated and dried (MgSO₄), and the solvent removed in vacuo. Purification by column chromatography (SiO₂, 0-3% MeOH in DCM) gave a clear glass (200 mg, 80%). This was dissolved in DCM (10 mL) and dry 3-chloroperoxybenzoic acid (148 mg, 0.86 mmol) was added. After stirring overnight, the mixture was diluted with DCM (20 mL) and washed with water (2×15 mL). The organic layer was separated and dried (MgSO₄), and the solvent was removed in vacuo. Purification by column chromatography (SiO₂, 0-3% MeOH in DCM) gave the desired sulfone (130 mg, 61%). To a solution of this material (111 mg, 0.22 mmol) in a 1:1 mixture of EtOH/CHCl₃ (2.3 mL) was added NaBH₄ (27.3 mg, 0.73 mmol) portionwise. After 1 h, water (7 mL) was added to the mixture. The solvents were removed in vacuo and the crude residue was redissolved in DCM and washed with water (2×10 mL). The organic layer was separated, dried (MgSO₄) and filtered, and the solvent removed in vacuo. Purification by column chromatography (SiO₂, 0-50% EtOAc in isohexane) gave the title compound (45 mg, 48%) as a white solid. δ_{H} (CDCl₃) 8.15 (1H, s), 8.09 (1H, s), 8.03 (1H, d, J 2.17 Hz), 7.57-7.50 (1H, m), 7.20 (1H, t, J 8.98 Hz), 7.17-7.10 (1H, m), 6.49 (1H, d, J 2.18 Hz), 6.06-6.01 (1H, m), 5.82-5.72 (1H, m), 4.32 (1H, d, J 17.29 Hz), 4.01 (1H, d, J 17.32 Hz), 3.96-3.88 (1H, m), 3.70-3.62 (1H, m), 3.57-3.47 (2H, m), 2.60 (3H, d, J 2.41 Hz), 1.71 (3H, d, J 6.76 Hz). LCMS (ES+) 421 (M+H)⁺, RT 2.83 minutes (Method 1).

### Example 12

### (S)-4-(7-Fluoro-8-methyl-3-{1-[2-(methylamino)pyrazolo[1,5-a][1,3,5]triazin-4-ylamino]ethyl}quinolin-2-yl)piperazin-2-one

TFA (2 mL) was added to a stirred solution of Intermediate 21 (161 mg, 0.40 mmol) in DCM (5 mL) and the mixture was allowed to stand at r.t. for 3 h before being concentrated in vacuo. The residue was dissolved in DCM (20 mL) and washed with 2M NaOH (2×10 mL) and water (10 mL). The organic layer was separated, dried (MgSO₄), filtered and concentrated in vacuo. To the residue (160 mg, 0.40 mmol) and DIPEA (0.28 mL, 1.62 mmol) dissolved in n-BuOH (3.3 mL) was added 4-chloro-2-(methylthio)-pyrazolo[1,5-a][1,3,5]triazine (162 mg, 0.81 mmol) and the reaction mixture was stirred at r.t. for 2 h. The solvent was removed in vacuo. The residue was redissolved in DCM (20 mL) and washed with water (2×15 mL). The organic layer was separated and dried (MgSO₄), and the solvent removed in vacuo. Purification by column chromatography (SiO₂, 0-3% MeOH in DCM) gave a clear glass (200 mg, 80%). This was dissolved in DCM (10 mL) and dry 3-chloroperoxybenzoic acid (148 mg, 0.86 mmol) was added. After stirring overnight, the mixture was diluted with DCM (20 mL) and washed with water (2×15 mL). The organic layer was separated and dried (MgSO₄), and the solvent was removed in vacuo. Purification by column chromatography (SiO₂, 0-3% MeOH in DCM) gave the desired sulfone (130 mg, 61%). To a solution of this material (80 mg, 0.16 mmol) in 1,4-dioxane (1.5 mL) was added methylamine (0.16 mL, 0.32 mmol; 2M in THF). The reaction mixture was heated at 50° C. for 2 h. After cooling, the solvent was removed in vacuo and the residue obtained was extracted with EtOAc (20 mL) and washed with saturated aqueous NaHCO₃ solution (2×10 mL). The organic layer was separated and dried (MgSO₄), and the solvent was removed in vacuo. Purification by preparative HPLC gave the title compound (10.9 mg, 15%) as a white solid. δ_{H} (CDCl₃) 8.07 (1H, s), 7.79 (1H, d, J 2.10 Hz), 7.50 (1H, dd, J 8.93, 6.04 Hz), 7.19-7.11 (1H, m), 6.82 (1H, d, J 7.20 Hz), 6.04 (1H, s), 5.98 (1H, s), 5.60-5.52 (1H, m), 4.95-4.58 (2H, m), 4.09 (2H, d, J 46.69 Hz), 3.60-3.49 (2H, m), 2.93 (3H, d, J 4.90 Hz), 2.58 (3H, d, J 2.36 Hz), 1.53 (3H, d, J 6.80 Hz). LCMS (ES+) 450 (M+H)⁺, RT 1.87 minutes (Method 2).

### Example 13

### 2-(4-{8-Chloro-3-[(1S)-1-(pyrazolo[1,5-a]pyrimidin-7-ylamino)ethyl]quinolin-2-yl}piperazin-1-yl)ethanol

Intermediate 4 (700 mg, 2.05 mmol), 1-(2-hydroxyethyl)piperazine (1 mL), n-BuOH (6 mL) and DIPEA (1 mL) were combined in a sealed tube and heated to 120° C. for 4 days. The reaction mixture was cooled, concentrated onto silica and purified by column chromatography (SiO₂, 0-100% EtOAc in isohexane) to give a clear gum (681 mg, 76%). This material (670 mg, 1.54 mmol), MeOH (5 mL) and 2N HCl in Et₂O (5 mL) were combined and stirred at r.t. for 24 h. The reaction mixture was concentrated to give a yellow solid (670 mg, quantitative). A portion of this material (65 mg, 0.159 mmol), n-BuOH (6 mL), DIPEA (1 mL) and 7-chloropyrazolo[1,5-a]pyrimidine (50 mg, 0.25 mmol) were combined in a sealed tube and heated under microwave irradiation to 140° C. for 1 h. The reaction mixture was then concentrated to dryness and purified by preparative HPLC to give the title compound (34 mg, 47%) as an off-white solid. δ_{H} (DMSO-d₆) 8.68 (1H, s), 8.46 (1H, d, J 7.72 Hz), 8.12 (2H, m), 7.84-7.78 (2H, m), 7.40 (1H, t, J 7.81 Hz), 6.44 (1H, d, J 2.27 Hz), 6.25 (1H, d, J 5.29 Hz), 5.15-5.07 (1H, m), 4.49 (1H, t, J 5.33 Hz), 3.62 (2H, q, J 5.72 Hz), 3.45-3.25 (4H, m), 2.90-2.80 (2H, m), 2.78-2.70 (2H, m), 2.58-2.51 (2H, m), 1.90 (3H, d, J 6.71 Hz). LCMS (ES+) 452/454 (M+H)⁺, RT 2.92 minutes (Method 1).

### Example 14

### (4-{8-Chloro-3-[(1S)-1-(pyrazolo[1,5-a]pyrimidin-7-ylamino)ethyl]quinolin-2-yl}piperazin-1-yl)acetic acid

Intermediate 22 (500 mg, 1.05 mmol), EtOH (5 mL) and 2N HCl in Et₂O (5 mL) were combined and stirred at r.t. for 4 days. The reaction mixture was then concentrated to give a yellow glass (526 mg, quantitative). A portion of this material (50 mg, 0.11 mmol), n-BuOH (6 mL), DIPEA (1 mL) and 7-chloropyrazolo[1,5-a]pyrimidine (50 mg, 0.25 mmol) were combined in a sealed tube and heated under microwave irradiation to 140° C. for 1 h. 15% NaOH solution (0.2 mL) was added to the reaction mixture, which was stirred at r.t. for 18 h. The reaction mixture was concentrated and purified by preparative HPLC to give the title compound (33.8 mg, 66%) as a tan glass. δ_{H} (DMSO-d₆) 8.68 (1H, s), 8.46 (1H, t, J 5.75 Hz), 8.12 (2H, m), 7.86-7.76 (2H, m), 7.40 (1H, t, J 7.81 Hz), 6.44 (1H, d, J 2.28 Hz), 6.26 (1H, d, J 5.27 Hz), 5.11 (1H, m), 3.48-3.40 (2H, m), 3.38-3.29 (2H, m), 3.30 (2H, s), 3.05-2.97 (2H, m), 2.94-2.86 (2H, m), 1.90 (3H, d, J 6.68 Hz). OH not seen. LCMS (ES+) 466/468 (M+H)⁺, RT 1.89 minutes (Method 2).

### Example 15

### Ethyl(4-{8-chloro-3-[(1S)-1-(pyrazolo[1,5-a]pyrimidin-7-ylamino)ethyl]quinolin-2-yl}piperazin-1-yl)acetate

Intermediate 22 (500 mg, 1.05 mmol), EtOH (5 mL) and 2N HCl in Et₂O (5 mL) were combined and stirred at r.t. for 4 days. The reaction mixture was then concentrated to give a yellow glass (526 mg, quantitative). A portion of this material (50 mg, 0.11 mmol), n-BuOH (6 mL), DIPEA (1 mL) and 7-chloropyrazolo[1,5-a]pyrimidine (50 mg, 0.25 mmol) were combined in a sealed tube and heated under microwave irradiation to 140° C. for 1 h. The reaction mixture was then concentrated and purified by preparative HPLC to give the title compound (39.3 mg, 72%) as a tan glass. δ_{H} (DMSO-d₆) 8.68 (1H, s), 8.47 (1H, d, J 7.70 Hz), 8.14-8.09 (2H, m), 7.86-7.76 (2H, m), 7.40 (1H, t, J 7.81 Hz), 6.44 (1H, d, J 2.27 Hz), 6.26 (1H, d, J 5.28 Hz), 5.14-5.05 (1H, m), 4.16 (2H, q, J 7.10 Hz), 3.47-3.38 (2H, m), 3.42 (2H, s), 3.38-3.29 (2H, m), 3.00-2.93 (2H, m), 2.90-2.82 (2H, m), 1.90 (3H, d, J 6.69 Hz), 1.25 (3H, t, J 7.09 Hz). LCMS (ES+) 494/496 (M+H)⁺, RT 3.50 minutes (Method 1).

### Example 16

### N-{[(3R)-1-{8-Chloro-3-[(1S)-1-(pyrazolo[1,5-a]pyrimidin-7-ylamino)ethyl]quinolin-2-yl}pyrrolidin-3-yl]methyl}acetamide

Intermediate 4 (700 mg, 2.05 mmol), (S)-1-[3-(aminomethyl)pyrrolidin-1-yl)]-ethanone (710 mg, 4.02 mmol), n-BuOH (6 mL) and DIPEA (1 mL) were combined in a sealed tube and heated to 130° C. for 10 days. The reaction mixture was cooled, concentrated onto silica and purified by column chromatography (SiO₂, 0-100% EtOAc in isohexane) to give tert-butyl(S)-1-{2-[(R)-3-(acetamidomethyl)pyrrolidin-1-yl]-8-chloro-quinolin-3-yl}ethylcarbamate (258 mg) as a white solid (from rearrangement of amine in situ) and tert-butyl(S)-1-(2-{[(S)-1-acetylpyrrolidin-3-ylmethyl]amino}-8-chloro-quinolin-3-yl)ethylcarbamate (189 mg) as a clear glass. tert-Butyl(S)-1-{2-[(R)-3-(acetamidomethyl)pyrrolidin-1-yl]-8-chloroquinolin-3-yl}ethylcarbamate (240 mg), MeOH (5 mL) and 2N HCl in Et₂O (5 mL) were combined and stirred at r.t. for 1 day. The reaction mixture was then concentrated in vacuo to give a yellow solid (250 mg). A portion of this solid (50 mg, 0.15 mmol), n-BuOH (6 mL), DIPEA (1 mL) and 7-chloro-pyrazolo[1,5-a]pyrimidine (50 mg, 0.25 mmol) were combined in a sealed tube and heated under microwave irradiation to 160° C. for 2 h. The reaction mixture was concentrated to dryness and purified by preparative HPLC to give the title compound (7.6 mg, 11%) as a tan solid. δ_{H} (DMSO-d₆) 8.46 (1H, s), 8.37 (1H, s), 8.25-8.22 (1H, m), 8.18-8.11 (2H, m), 7.77 (1H, d), 7.71 (1H, d), 7.25 (1H, t, J 7.77 Hz), 6.55 (1H, d, J 2.28 Hz), 6.12-6.06 (1H, m), 5.34 (1H, m), 3.99-3.84 (3H, m), 3.78-3.65 (1H, m), 3.33-3.21 (2H, m), 2.61-2.52 (1H, m), 2.24-2.14 (1H, m), 1.95-1.81 (7H, m). LCMS (ES+) 464/466 (M+H)⁺, RT 2.91 minutes (Method 1).

### Example 17

### 1-{7-Fluoro-8-methyl-3-[(1S)-1-(pyrazolo[1,5-a]pyrimidin-7-ylamino)ethyl]quinolin-2-yl}piperidine-4-carboxamide

Intermediate 15 (700 mg, 2.06 mmol), piperidine-4-carboxylic acid amide (0.5 g, 3.9 mmol), n-BuOH (10 mL) and DIPEA (4 mL) were combined in a sealed tube and heated to 130° C. for 12 days. The reaction mixture was cooled, concentrated onto silica and purified by column chromatography (SiO₂, 0-10% MeOH in EtOAc) to give a clear gum. This material, MeOH (10 mL) and 2N HCl in Et₂O (7 mL) were combined and stirred at r.t. for 24 h. The reaction mixture was concentrated to give a yellow solid. A portion of this material (60 mg), n-BuOH (6 mL), DIPEA (1 mL) and 7-chloro-pyrazolo[1,5-a]pyrimidine (50 mg, 0.25 mmol) were combined in a sealed tube and heated under microwave irradiation to 160° C. for 2 h. The reaction mixture was then concentrated to dryness and purified by preparative HPLC to give the title compound (25.2 mg, 34%) as a beige solid. δ_{H} (DMSO-d₆) 8.60 (1H, s), 8.40 (1H, d, J 7.74 Hz), 8.13-8.08 (2H, m), 7.70 (1H, dd, J 8.94, 6.31 Hz), 7.38 (1H, s), 7.30 (1H, t, J 9.12 Hz), 6.87 (1H, s), 6.43 (1H, d, J 2.28 Hz), 6.25 (1H, d, J 5.29 Hz), 5.07 (1H, m), 3.69 (1H, d, J 12.66 Hz), 3.56 (1H, d, J 12.72 Hz), 3.18 (1H, t, J 12.09 Hz), 2.92 (1H, t, J 12.09 Hz), 2.56 (3H, s), 2.47-2.37 (1H, m), 2.10-1.97 (2H, m), 1.98-1.86 (5H, m). LCMS (ES+) 448 (M+H)⁺, RT 2.34 minutes (Method 2).

### Example 18

### N-[(3S)-1-{7-Fluoro-8-methyl-3-[(1S)-1-(pyrazolo[1,5-a]pyrimidin-7-ylamino)ethyl]-quinolin-2-yl}pyrrolidin-3-yl]cyclopropanecarboxamide

(S)-(−)-3-Amino-1-pyrrolidinecarboxylic acid tert-butyl ester (500 mg, 2.68 mmol), DCM (30 mL), DIPEA (2 mL) and cyclopropanecarbonyl chloride (0.275 mL, 3 mmol) were combined at r.t. under a nitrogen atmosphere. The reaction mixture was stirred for 1 day, then diluted with DCM (50 mL) and washed with water (50 mL). The organic layer was separated, dried (MgSO₄) and concentrated in vacuo to give a brown oil. This oil, MeOH (10 mL) and 2N HCl in Et₂O (5 mL) were stirred at r.t. for 3 days. The reaction mixture was then concentrated in vacuo. The resulting material, Intermediate 15 (500 mg, 1.48 mmol), n-BuOH (16 mL) and DIPEA (2 mL) were combined in a sealed tube and heated to 130° C. for 15 days. The reaction mixture was cooled, concentrated onto silica and purified by column chromatography (SiO₂, 0-100% EtOAc in isohexane) to give a tan solid. This material, MeOH (10 mL) and 2N HCl in Et₂O (7 mL) were combined and stirred at r.t. for 24 h. The reaction mixture was concentrated to give a brown glass. A portion of this material (50 mg, 0.127 mmol), n-BuOH (6 mL), DIPEA (1 mL) and 7-chloropyrazolo[1,5-a]pyrimidine (50 mg, 0.25 mmol) were combined in a sealed tube and heated under microwave irradiation to 160° C. for 2 h. The reaction mixture was then concentrated to dryness and purified by preparative HPLC to give the title compound (32.3 mg, 54%) as a brown glass. δ_{H} (DMSO-d₆) 8.44 (1H, d, J 6.91 Hz), 8.38 (1H, d, J 6.91 Hz), 8.25 (1H, s), 8.17 (1H, d, J 2.27 Hz), 8.10 (1H, d, J 5.21 Hz), 7.57 (1H, dd, J 8.84, 6.41 Hz), 7.10 (1H, t, J 9.10 Hz), 6.49 (1H, d, J 2.27 Hz), 6.06 (1H, d, J 5.28 Hz), 5.27 (1H, m), 4.50-4.42 (1H, m), 4.07-3.94 (2H, m), 3.82-3.74 (1H, m), 3.60 (1H, dd, J 10.47, 5.21 Hz), 2.51 (3H, s), 2.32-2.23 (1H, m), 2.04-1.94 (1H, m), 1.77 (3H, d, J 6.54 Hz), 1.66-1.58 (1H, m), 0.77-0.67 (4H, m). LCMS (ES+) 474 (M+H)⁺, RT 2.51 minutes (Method 2).

### Example 19

### N-[(3R)-1-{8-Chloro-3-[(1S)-1-(pyrazolo[1,5-a]pyrimidin-7-ylamino)ethyl]quinolin-2-yl}pyrrolidin-3-yl]-2-hydroxyacetamide

(R)-(+)-3-Amino-1-pyrrolidinecarboxylic acid tert-butyl ester (400 mg, 2.15 mmol), DCM (30 mL), DIPEA (2 mL), and acetoxyacetyl chloride (0.247 mL, 2.3 mmol) were combined at r.t. under nitrogen atmosphere. The reaction mixture was stirred for 7 days, then diluted with DCM (50 mL) and washed with water (30 mL). The organic layer was separated, dried (MgSO₄) and concentrated in vacuo to give a tan oil. This material, MeOH (10 mL) and 2N HCl in Et₂O (4 mL) were stirred at r.t. for 1 day. The reaction mixture was concentrated in vacuo. The resulting material, Intermediate 4 (400 mg, 1.17 mmol), n-BuOH (14 mL) and DIPEA (2 mL) were combined in a sealed tube and heated to 130° C. for 13 days. The reaction mixture was cooled, concentrated onto silica and purified by column chromatography (SiO₂, 0-100% EtOAc in isohexane then 10% MeOH in EtOAc) to give an off-white foam. This material, MeOH (10 mL) and 2N HCl in Et₂O (6 mL) were combined and stirred at r.t. for 24 h. The reaction mixture was concentrated to give a yellow solid. A portion of this material (50 mg, 0.13 mmol), n-BuOH (6 mL), DIPEA (1 mL) and 7-chloropyrazolo[1,5-a]pyrimidine (50 mg, 0.25 mmol) were combined in a sealed tube and heated under microwave irradiation to 160° C. for 2 h. The reaction mixture was then concentrated to dryness and purified by preparative HPLC to give the title compound (10.3 mg, 17%) as an off-white solid. δ_{H} (DMSO-d₆) 8.43 (1H, d, J 7.03 Hz), 8.32 (1H, s), 8.20-8.06 (3H, m), 7.72 (1H, d, J 7.48 Hz), 7.65 (1H, d, J 8.00 Hz), 7.20 (1H, t, J 7.77 Hz), 6.49 (1H, d, J 2.32 Hz), 6.03 (1H, d, J 5.23 Hz), 5.50 (1H, s), 5.27 (1H, m), 4.59-4.51 (1H, m), 4.00-3.84 (6H, m), 2.24 (1H, m), 2.11 (1H, m), 1.80 (3H, d, J 6.51 Hz). LCMS (ES+) 466/468 (M+H)⁺, RT 2.79 minutes (Method 1).

### Example 20

### (4-{7-Fluoro-8-methyl-3-[(1S)-1-(pyrazolo[1,5-a]pyrimidin-7-ylamino)ethyl]quinolin-2-yl}piperazin-1-yl)acetic acid

Intermediate 15 (700 mg, 2.06 mmol), ethyl 1-piperazineacetate (0.5 g), n-BuOH (10 mL) and DIPEA (2 mL) were combined in a sealed tube and heated to 130° C. for 12 days. The reaction mixture was cooled, concentrated onto silica and purified by column chromatography (SiO₂, 0-100% EtOAc in isohexane). The resulting material, EtOH (7 mL) and 2N HCl in Et₂O (5 mL) were combined and stirred at r.t. for 3 days. The reaction mixture was concentrated to give a pale yellow solid. A portion of this material (50 mg), n-BuOH (6 mL), DIPEA (1 mL) and 7-chloropyrazolo[1,5-a]pyrimidine (50 mg, 0.25 mmol) were combined in a sealed tube and heated under microwave irradiation to 160° C. for 2 h. 15% NaOH solution (0.2 mL) was then added to the reaction mixture which was stirred at r.t. for 3 days. The reaction mixture was then concentrated to dryness and purified by preparative HPLC to give the title compound (32.9 mg, 60%) as a beige solid. δ_{H} (DMSO-d₆) 8.67 (1H, s), 8.47 (1H, d, J 8.21 Hz), 8.19-8.14 (2H, m), 7.77 (1H, dd, J 8.96, 6.25 Hz), 7.36 (1H, t, J 9.11 Hz), 6.49 (1H, d, J 2.27 Hz), 6.34 (1H, d, J 5.30 Hz), 5.16 (1H, m), 3.50-3.30 (6H, m), 3.08-2.91 (4H, m), 2.62 (3H, s), 1.94 (3H, d, J 6.71 Hz). OH not seen. LCMS (ES+) 464 (M+H)⁺, RT 2.37 minutes (Method 1).

### Example 21

### 1-(4-{8-Chloro-3-[(1S)-1-(pyrazolo[1,5-a]pyrimidin-7-ylamino)ethyl]quinolin-2-yl}piperazin-1-yl)-2-hydroxyethanone

Intermediate 4 (700 mg, 2.05 mmol), 2-hydroxy-1-(piperazin-1-yl)ethanone hydrochloride, n-BuOH (10 mL) and DIPEA (4 mL) were combined in a sealed tube and heated to 130° C. for 7 days. The reaction mixture was cooled, concentrated onto silica and purified by column chromatography (SiO₂, 0-10% MeOH in EtOAc) to give a yellow gum. This material, MeOH (5 mL) and 2N HCl in Et₂O (5 mL) were combined and stirred at r.t. for 24 h. The reaction mixture was concentrated to give a yellow glass. A portion of this material (50 mg, 0.13 mmol), n-BuOH (6 mL), DIPEA (1 mL) and 7-chloropyrazolo[1,5-a]pyrimidine (50 mg, 0.25 mmol) were combined in a sealed tube and heated under microwave irradiation to 160° C. for 2 h. The reaction mixture was concentrated to dryness and purified by preparative HPLC to give the title compound (8.8 mg, 15%) as a yellow solid. δ_{H} (DMSO-d₆) 8.67 (1H, s), 8.40 (1H, d, J 7.79 Hz), 8.09 (2H, t, J 2.40 Hz), 7.82-7.75 (2H, m), 7.38 (1H, t, J 7.82 Hz), 6.40 (1H, d, J 2.27 Hz), 6.21 (1H, d, J 5.29 Hz), 5.13 (1H, m), 4.64 (1H, t, J 5.50 Hz), 4.21-4.14 (2H, m), 3.86-3.56 (4H, m), 3.41-3.19 (4H, m), 1.85 (3H, d, J 6.70 Hz). LCMS (ES+) 466/468 (M+H)⁺, RT 2.24 minutes (Method 2).

### Example 22

### 1-(4-{8-Chloro-3-[(1S)-1-(pyrazolo[1,5-a]pyrimidin-7-ylamino)ethyl]quinolin-2-yl}piperazin-1-yl)-2-(dimethylamino)ethanone formic acid salt

Intermediate 4 (700 mg, 2.05 mmol), 2-(dimethylamino)-1-(piperazin-1-yl)-ethanone (500 mg), n-BuOH (10 mL) and DIPEA (4 mL) were combined in a sealed tube and heated to 130° C. for 10 days. The reaction mixture was cooled, concentrated onto silica and purified by column chromatography (SiO₂, 0-10% MeOH in EtOAc) to give a cream solid. This material, MeOH (6 mL) and 2N HCl in Et₂O (6 mL) were combined and stirred at r.t. for 24 h. The reaction mixture was concentrated to give a yellow foam. A portion of this material (50 mg, 0.12 mmol), n-BuOH (6 mL), DIPEA (1 mL) and 7-chloropyrazolo[1,5-a]pyrimidine (50 mg, 0.25 mmol) were combined in a sealed tube and heated under microwave irradiation to 160° C. for 2 h. The reaction mixture was concentrated to dryness and purified by preparative HPLC to give the title compound (7.1 mg, 11%) as a clear glass. δ_{H} (DMSO-d₆) 8.71 (1H, s), 8.45 (1H, d, J 7.71 Hz), 8.27 (1H, s), 8.13 (2H, m), 7.83 (2H, t, J 8.63 Hz), 7.42 (1H, t, J 7.81 Hz), 6.44 (1H, d, J 2.29 Hz), 6.26 (1H, d, J 5.27 Hz), 5.18 (1H, m), 4.04-3.74 (4H, m), 3.49-3.14 (6H, m), 2.26 (6H, s), 1.91 (3H, d, J 6.64 Hz). LCMS (ES+) 493/495 (M+H)⁺, RT 3.05 minutes (Method 1).

### Example 23

### 1-{7-Fluoro-8-methyl-3-[(1S)-1-(pyrazolo[1,5-a]pyrimidin-7-ylamino)ethyl]quinolin-2-yl}piperidine-4-carboxylic acid

Intermediate 15 (700 mg, 2.06 mmol), piperidine-4-carboxylic acid methyl ester hydrochloride (500 mg, 2.78 mmol), n-BuOH (10 mL) and DIPEA (4 mL) were combined in a sealed tube and heated to 130° C. for 12 days. The reaction mixture was cooled, concentrated onto silica and purified by column chromatography (SiO₂, 0-10% MeOH in EtOAc). The resulting material, MeOH (10 mL) and 2N HCl in Et₂O (6 mL) were combined and stirred at r.t. for 24 h. The reaction mixture was concentrated to give a tan foam. A portion of this material (50 mg), n-BuOH (6 mL), DIPEA (1 mL) and 7-chloropyrazolo[1,5-a]pyrimidine (50 mg, 0.25 mmol) were combined in a sealed tube and heated under microwave irradiation to 160° C. for 2 h. 15% NaOH solution (0.2 mL) was added to the reaction mixture, which was stirred at r.t. for 3 days. The reaction mixture was concentrated to dryness and purified by preparative HPLC to give the title compound (21.8 mg, 43%) as a tan solid. δ_{H} (DMSO-d₆) 8.60 (1H, s), 8.40 (1H, m), 8.11 (2H, m), 7.70 (1H, dd, J 8.93, 6.31 Hz), 7.30 (1H, t, J 9.12 Hz), 6.43 (1H, d, J 2.27 Hz), 6.25 (1H, d, J 5.29 Hz), 5.07 (1H, m), 3.63 (1H, d, J 12.90 Hz), 3.53 (1H, d, J 13.17 Hz), 3.26-3.17 (1H, m), 2.99 (1H, dt, J 13.20, 6.76 Hz), 2.55 (3H, s), 2.14 (1H, d, J 13.29 Hz), 2.06 (2H, d, J 5.12 Hz), 1.97-1.87 (4H, m). OH not seen. LCMS (ES+) 449 (M+H)⁺, RT 2.71 minutes (Method 1).

### Example 24

### 2-(Dimethylamino)-1-(4-{7-fluoro-8-methyl-3-[(1S)-1-(pyrazolo[1,5-a]pyrimidin-7-ylamino)ethyl]quinolin-2-yl}piperazin-1-yl)ethanone

Intermediate 15 (700 mg, 2.06 mmol), 2-(dimethylamino)-1-(piperazin-1-yl)-ethanone (500 mg), n-BuOH (10 mL) and DIPEA (2 mL) were combined in a sealed tube and heated to 130° C. for 13 days. The reaction mixture was cooled, concentrated onto silica and purified by column chromatography (SiO₂, 0-10% MeOH in EtOAc) to give a white solid. This material, MeOH (8 mL) and 2N HCl in Et₂O (4 mL) were combined and stirred at r.t. for 2 days. The reaction mixture was concentrated to give a yellow foam. A portion of this material (50 mg, 0.12 mmol), n-BuOH (6 mL), DIPEA (1 mL) and 7-chloropyrazolo[1,5-a]pyrimidine (50 mg, 0.25 mmol) were combined in a sealed tube and heated to 130° C. for 16 h. The reaction mixture was then concentrated to dryness and purified by preparative HPLC to give the title compound (28 mg, 48%) as a tan glass. δ_{H} (DMSO-d₆) 8.64 (1H, s), 8.41 (1H, d, J 7.80 Hz), 8.13 (2H, m), 7.74 (1H, dd, J 8.95, 6.28 Hz), 7.33 (1H, t, J 9.13 Hz), 6.44 (1H, d, J 2.27 Hz), 6.28 (1H, d, J 5.30 Hz), 5.21-5.13 m), 4.16-3.73 (4H, m), 3.42-3.17 (6H, m), 2.56 (3H, s), 2.25 (6H, s), 1.89 (3H, d, J 6.71 Hz). LCMS (ES+) 491 (M+H)⁺, RT 3.23 minutes (Method 1).

### Example 25

### 1-{7-Fluoro-8-methyl-3-[(1S)-1-(pyrazolo[1,5-a]pyrimidin-7-ylamino)ethyl]quinolin-2-yl}-N-methylpiperidine-4-carboxamide

Intermediate 15 (700 mg, 2.06 mmol), piperidine-4-carboxylic acid methylamide (426 mg, 3 mmol), n-BuOH (10 mL) and DIPEA (3 mL) were combined in a sealed tube and heated to 130° C. for 20 days. The reaction mixture was cooled, concentrated onto silica and purified by column chromatography (SiO₂, 0-10% MeOH in EtOAc) to give a white solid. This material, MeOH (10 mL) and 2N HCl in Et₂O (5 mL) were combined and stirred at r.t. for 4 days. The reaction mixture was concentrated to give a yellow solid. A portion of this material (50 mg, 0.12 mmol), n-BuOH (6 mL), DIPEA (1 mL) and 7-chloropyrazolo[1,5-a]pyrimidine (50 mg, 0.25 mmol) were combined in a sealed tube and heated to 130° C. for 16 h. The reaction mixture was then concentrated to dryness and purified by preparative HPLC to give the title compound (36.9 mg, 67%) as a brown glass. δ_{H} (DMSO-d₆) 8.60 (1H, s), 8.41 (1H, m), 8.13-8.08 (2H, m), 7.85 (1H, m), 7.70 (1H, dd, J 8.94, 6.31 Hz), 7.30 (1H, t, J 9.12 Hz), 6.43 (1H, d, J 2.27 Hz), 6.25 (1H, d, J 5.30 Hz), 5.07 (1H, m), 3.70 (1H, d, J 12.74 Hz), 3.57 (1H, d, J 12.76 Hz), 3.21-3.14 (1H, m), 2.96-2.88 (1H, t, J 12.16 Hz), 2.65 (3H, d, J 4.53 Hz), 2.55 (3H, s), 2.47-2.37 (1H, m), 2.16-1.87 (7H, m). LCMS (ES+) 462 (M+H)⁺; RT 3.72 minutes (Method 1).

### Example 26

### 1-{8-Chloro-3-[(1S)-1-(pyrazolo[1,5-a]pyrimidin-7-ylamino)ethyl]quinolin-2-yl}-N-methylpiperidine-4-carboxamide

Intermediate 4 (700 mg, 2.06 mmol), piperidine-4-carboxylic acid methylamide (426 mg, 3 mmol), n-BuOH (10 mL) and DIPEA (3 mL) were combined in a sealed tube and heated to 130° C. for 10 days. The reaction mixture was cooled, concentrated onto silica and purified by column chromatography (SiO₂, 0-10% MeOH in EtOAc) to give a white solid. This material, MeOH (10 mL) and 2N HCl in Et₂O (5 mL) were combined and stirred at r.t. for 3 days. The reaction mixture was concentrated to give a yellow solid. A portion of this material (50 mg, 0.12 mmol), n-BuOH (6 mL), DIPEA (1 mL) and 7-chloropyrazolo[1,5-a]pyrimidine (50 mg, 0.25 mmol) were combined in a sealed tube and heated to 130° C. for 16 h. The reaction mixture was then concentrated to dryness and purified by preparative HPLC to give the title compound (33 mg, 59%) as a tan solid. δ_{H} (DMSO-d₆) 8.67 (1H, s), 8.44 (1H, d, J 7.70 Hz), 8.14-8.09 (2H, m), 7.88-7.76 (3H, m), 7.39 (1H, t, J 7.81 Hz), 6.44 (1H, d, J 2.27 Hz), 6.23 (1H, d, J 5.27 Hz), 5.12-5.04 (1H, m), 3.73 (1H, d, J 12.82 Hz), 3.61 (1H, d, J 12.75 Hz), 3.22-3.15 (2H, m), 2.96 (1H, t, J 12.22 Hz), 2.65 (3H, d, J 4.50 Hz), 2.44 (1H, m), 2.18-1.95 (2H, m), 1.95-1.82 (4H, m). LCMS (ES+) 464/466 (M+H)⁺, RT 2.37 minutes (Method 2).

### Example 27

### (S)—N,N-Dimethyl-2-(4-{7-fluoro-8-methyl-3-[1-(pyrazolo[1,5-a]pyrimidin-7-ylamino)-ethyl]quinolin-2-yl}piperazin-1-yl)acetamide

Intermediate 15 (822 mg, 2.04 mmol), N,N-dimethyl-2-(piperazin-1-yl)acetamide (1.75 g, 10.2 mmol), DIPEA (1.78 mL, 10.2 mmol) and n-BuOH (5.0 mL) were combined in a sealed tube and heated to 140° C. for 48 h. After cooling, the reaction mixture was dissolved in a 1:1 mixture of EtOAc and Et₂O (400 mL) and washed with saturated brine (3×100 mL). The organic layer was dried (MgSO₄) and concentrated in vacuo. Purification by column chromatography (SiO₂, 0-100% EtOAc in isohexane) gave an oil (800 mg, 83%). To this material (400 mg, 0.84 mmol) in DCM (5 mL) was added TFA (1 mL) and the mixture was allowed to stand at r.t. for 3 h before being concentrated in vacuo. The residue was dissolved in DCM (100 mL) and washed with 2M NaOH solution (2×30 mL) and water (30 mL). The organic layer was separated, dried (MgSO₄), filtered and concentrated in vacuo. To a portion of the resulting material (132 mg, 0.35 mmol) dissolved in n-BuOH (2.5 mL) and DIPEA (0.19 mL, 1.05 mmol) was added 7-chloropyrazolo[1,5-a]pyrimidine (71 mg, 0.46 mmol) and the mixture was heated to 130° C. overnight in a sealed tube. The reaction mixture was cooled and the solvent removed in vacuo. The residue was redissolved in DCM (50 mL) and washed with water (10 mL) and brine (10 mL). The organic layer was separated, dried (MgSO₄) and filtered, and the solvent was removed in vacuo. Purification by preparative HPLC afforded the title compound (104.1 mg, 58%) as a white solid. δ_{H} (CDCl₃) 8.12 (1H, d, J 5.22 Hz), 8.09 (1H, s), 8.00 (1H, d, J 2.32 Hz), 7.49 (1H, dd, J 8.93, 6.02 Hz), 7.19-7.09 (1H, m), 6.88 (1H, d, J 6.73 Hz), 6.49 (1H, d, J 2.32 Hz), 6.04 (1H, d, J 5.25 Hz), 5.21-5.10 (1H, m), 3.43-3.38 (4H, m), 3.35 (2H, d, J 5.15 Hz), 3.13 (3H, s), 2.98 (2H, s), 3.02-2.78 (3H, m), 2.75-2.65 (2H, m), 2.60 (3H, d, J 2.40 Hz), 1.83 (3H, d, J 6.73 Hz). LCMS (ES+) 491 (M+H)⁺, RT 1.89 minutes (Method 2).

### Example 28

### 4-{8-Methyl-3-[(pyrazolo[1,5-a]pyrimidin-7-ylamino)methyl]quinolin-2-yl}piperazin-2-one

Intermediate 24 (60 mg, 0.22 mmol) in NMP (1.0 mL) was treated with 7-chloro-pyrazolo[1,5-a]pyrimidine (41 mg, 0.27 mmol) and DIPEA (0.20 mL, 1.11 mmol). The mixture was heated at 120° C. under microwave irradiation for 1 h. Purification by preparative HPLC gave the title compound (38.0 mg, 44%) as a beige solid. δ_{H} (CDCl₃) 8.19 (1H, d, J 5.13 Hz), 8.13-8.06 (1H, m), 8.03 (1H, d, J 2.31 Hz), 7.53 (2H, t, J 8.30 Hz), 7.33 (1H, t, J 7.56 Hz), 7.18 (1H, t, J 5.93 Hz), 6.55 (1H, d, J 2.31 Hz), 6.25 (1H, s), 5.88 (1H, d, J 5.15 Hz), 4.79 (2H, d, J 5.94 Hz), 4.11 (2H, s), 3.59 (4H, s), 2.72 (3H, s). LCMS (ES+) 388 (M+H)⁺, RT 2.04 minutes (Method 2).

### Example 29

### (S)-2,2-Dimethyl-1-(4-{7-fluoro-8-methyl-3-[1-(pyrazolo[1,5-a]pyrimidin-7-ylamino)-ethyl]quinolin-2-yl}piperazin-1-yl)propan-1-one

To a solution of Intermediate 25 (60 mg, 0.16 mmol) in NMP (1.2 mL) were added DIPEA (0.14 mL) and 7-chloropyrazolo[1,5-a]pyrimidine (30 mg, 0.194 mmol) and the resulting solution was heated under microwave irradiation at 150° C. for 1 h. Purification by preparative HPLC afforded the title compound (42 mg, 54%) as an off-white solid. δ_{H} (DMSO-d₆) 8.64 (1H, s), 8.43 (1H, d, J 7.76 Hz), 8.13 (2H, m), 7.74 (1H, dd, J 8.96, 6.23 Hz), 7.33 (1H, t, J 9.12 Hz), 6.44 (1H, d, J 2.27 Hz), 6.28 (1H, d, J 5.30 Hz), 5.17 (1H, m), 4.01-3.93 (2H, m), 3.91-3.83 (2H, m), 3.20-3.40 (4H, m), 2.58 (3H, s), 1.89 (3H, d, J 6.70 Hz), 1.30 (9H, s). LCMS (ES+) 490 (M+H)⁺, RT 2.85 minutes (Method 2).

### Example 30

### 4-{8-Methyl-3-[(pyrazolo[1,5-a][1,3,5]triazin-4-ylamino)methyl]quinolin-2-yl}piperazin-2-one

Intermediate 38 (170 mg, 0.39 mmol) in DCM (10 mL) was treated with 3-chloro-peroxybenzoic acid (176 mg, 0.78 mmol; 77% w/w in water) and stirred for 3 h. The reaction mixture was partitioned between water (20 mL) and DCM (20 mL). The organic layer was washed with saturated aqueous NaHCO₃ solution (20 mL) and brine (20 mL), separated, dried (phase separation cartridge) and concentrated in vacuo to give a beige solid (130 mg, 71%). A portion of this material (90 mg, 0.193 mmol) in a 1:1 mixture of EtOH/CHCl₃ (3.0 mL) was treated with NaBH₄ (8.0 mg, 0.193 mmol) and stirred for 30 minutes. The reaction mixture was partitioned between EtOAc (6 mL) and H₂O (6 mL). The organic layer was washed with H₂O (6 mL) and brine (6 mL), separated, dried (MgSO₄) and concentrated in vacuo. Purification by chromatography (SiO₂, 5-10% MeOH in EtOAc) gave the title compound (40 mg, 54%) as a white solid. δ_{H} (CDCl₃) 8.24 (1H, s), 8.09 (1H, s), 8.00 (1H, d, J 2.16 Hz), 7.61-7.45 (3H, m), 7.36-7.30 (1H, m), 6.48 (1H, d, J 2.16 Hz), 5.99 (1H, s), 5.08 (2H, d, J 5.95 Hz), 4.12 (2H, s), 3.60 (4H, m), 2.71 (3H, s). LCMS (ES+) 389 (M+H)⁺, RT 2.74 minutes (Method 2).

### Example 31

### 4-{3-[(2-Aminopyrazolo[1,5-a][1,3,5]triazin-4-ylamino)methyl]-8-methylquinolin-2-yl}piperazin-2-one

Intermediate 38 (170 mg, 0.39 mmol) in DCM (10 mL) was treated with 3-chloro-peroxybenzoic acid (176 mg, 0.78 mmol; 77% w/w in H₂O) and stirred for 3 h. The reaction mixture was partitioned between water (20 mL) and DCM (20 mL). The organic layer was washed with saturated aqueous NaHCO₃ solution (20 mL) and brine (20 mL), separated, dried (phase separation cartridge) and concentrated in vacuo to give a beige solid (130 mg, 71%). A portion of this material (40 mg, 0.086 mmol) in 1,4-dioxane (1.5 mL) was treated with aqueous ammonia solution (0.2 mL) and heated at 60° C. for 16 h. The solvent was removed in vacuo and the residue was purified by column chromatography (SiO₂, 0-20% MeOH in EtOAc) to give the title compound (10.0 mg, 29%) as a white solid. δ_{H} (DMSO-d₆) 8.89 (1H, t, J 6.05 Hz), 8.13 (1H, s), 7.99 (1H, s), 7.88 (1H, s), 7.70 (1H, d, J 7.97 Hz), 7.52 (1H, d, J 7.04 Hz), 7.32 (1H, t, J 7.55 Hz), 6.51 (2H, s), 5.81 (1H, s), 4.83 (2H, d, J 6.00 Hz), 4.05 (2H, s), 3.55 (2H, m), 3.42 (2H, m), 2.67 (3H, s). LCMS (ES+) 404 (M+H)⁺, RT 2.11 minutes (Method 2).

### Example 32

### (S)—N-(1-{7-Fluoro-8-methyl-2-[4-(pyridin-2-yl)piperazin-1-yl]quinolin-3-yl}ethyl)-pyrazolo[1,5-a]pyrimidin-7-amine

To a solution of Intermediate 15 (500 mg, 1.48 mmol) in NMP (6 mL) were added 1-(pyridin-2-yl)piperazine (500 mg, 3.00 mmol) and DIPEA (1.3 mL) and the resulting mixture was heated at 140° C. for 16 h. The reaction mixture was taken up in EtOAc (150 mL) and water (50 mL) and the organic layer was washed with water (2×50 mL) and brine (50 mL). The organic layer was separated, dried (phase separation cartridge), and the solvent removed in vacuo. Purification by column chromatography (SiO₂, 10-20% EtOAc in isohexane) gave a beige solid (530 mg, 77%). To a solution of this material (530 mg, 1.13 mmol) in DCM (6 mL) was added TFA (3 mL) and the resulting solution stirred at r.t. for 15 minutes. The solvents were removed in vacuo. The residue was dissolved in MeOH (5 mL) and placed on an SCX cartridge, washed (MeOH), eluted (7M ammonia in MeOH) and concentrated in vacuo. The solvent was concentrated in vacuo to afford a white solid (416 mg, 100%). To a portion of this material (60 mg, 0.16 mmol) in NMP (1.2 mL) were added DIPEA (0.14 mL) and 7-chloropyrazolo[1,5-a]pyrimidine (30 mg, 0.194 mmol) and the resulting solution was heated under microwave irradiation at 150° C. for 1 h. Purification by preparative HPLC afforded the title compound (20 mg, 26%) as an off-white solid. δ_{H} (DMSO-d₆) 8.66 (1H, s), 8.43 (1H, d, J 7.76 Hz), 8.21 (1H, dd, J 4.94, 1.93 Hz), 8.14-8.11 (2H, m), 7.74 (1H, dd, J 8.97, 6.26 Hz), 7.63 (1H, dd, J 8.58, 7.09 Hz), 7.33 (1H, t, J 9.12 Hz), 6.98 (1H, d, J 8.63 Hz), 6.73 (1H, dd, J 7.09, 4.91 Hz), 6.44 (1H, s), 6.33 (1H, d, J 5.30 Hz), 5.21 (1H, m), 3.94-3.87 (2H, m), 3.84-3.77 (2H, m), 3.52-3.43 (4H, m), 2.58 (3H, m), 1.92 (3H, d, J 6.71 Hz). LCMS (ES+) 483 (M+H)⁺, RT 2.33 minutes (Method 2).

### Example 33

### (S)-(Cyclopropyl)(4-{7-fluoro-8-methyl-3-[1-(pyrazolo[1,5-a]pyrimidin-7-ylamino)-ethyl]quinolin-2-yl}piperazin-1-yl)methanone

Following the procedure described for Example 32, Intermediate 15 (500 mg, 1.48 mmol) and (cyclopropyl)(piperazin-1-yl)methanone (455 mg, 2.95 mmol) gave the desired product (527 mg, 100%). A portion of this material (57 mg, 0.16 mmol) and 7-chloropyrazolo[1,5-a]pyrimidine (30 mg, 0.194 mmol) afforded the title compound (40 mg, 50%) as a pink solid. δ_{H} (DMSO-d₆) 8.65 (1H, s), 8.41 (1H, d, J 7.83 Hz), 8.16-8.11 (2H, m), 7.75 (1H, dd, J 8.95, 6.27 Hz), 7.34 (1H, t, J 9.13 Hz), 6.44 (1H, s), 6.30 (1H, d, J 5.30 Hz), 5.19 (1H, m), 4.20-3.75 (4H, m), 3.30-3.10 (4H, m), 2.57 (3H, s), 2.14-2.07 (1H, m), 1.89 (3H, d, J 6.71 Hz), 0.86-0.76 (4H, m). LCMS (ES+) 474 (M+H)⁺, RT 2.60 minutes (Method 2).

### Example 34

### (S)—N-(1-{7-Fluoro-8-methyl-2-[4-(pyrazin-2-yl)piperazin-1-yl]quinolin-3-yl}ethyl)-pyrazolo[1,5-a]pyrimidin-7-amine

Following the procedure described for Example 32, Intermediate 15 (500 mg, 1.48 mmol) and 2-(piperazin-1-yl)pyrazine (485 mg, 2.95 mmol) gave the desired product (542 mg, 100%). A portion of this material (59 mg, 0.16 mmol) and 7-chloropyrazolo[1,5-a]-pyrimidine (30 mg, 0.194 mmol) afforded the title compound (27 mg, 35%) as a beige solid. δ_{H} (DMSO-d₆) 8.67 (1H, s), 8.47 (1H, s), 8.43 (1H, d, J 8.0 Hz), 8.20 (1H, s), 8.12 (2H, d, J 8.0 Hz), 7.94 (1H, s), 7.75 (1H, dd, J 8.96, 6.25 Hz), 7.34 (1H, t, J 9.13 Hz), 6.44 (1H, s), 6.32 (1H, d, J 5.30 Hz), 5.22 (1H, m), 4.03-3.96 (2H, m), 3.92-3.85 (2H, m), 3.57-3.30 (4H, m), 2.58 (3H, s), 1.91 (3H, d, J 6.70 Hz). LCMS (ES+) 484 (M+H)⁺, RT 3.67 minutes (Method 2).

### Example 35

### (S)-1-(4-{7-Fluoro-8-methyl-3-[1-(pyrazolo[1,5-a]pyrimidin-7-ylamino)ethyl]quinolin-2-yl}piperazin-1-yl)propan-1-one

Following the procedure described for Example 32, Intermediate 15 (500 mg, 1.48 mmol) and 1-(piperazin-1-yl)propan-1-one (420 mg, 2.95 mmol) gave the desired product (510 mg, 100%). A portion of this material (55 mg, 0.16 mmol) and 7-chloro-pyrazolo[1,5-a]pyrimidine (30 mg, 0.194 mmol) afforded the title compound (20 mg, 28%) as a pink solid. δ_{H} (DMSO-d₆) 8.64 (1H, s), 8.42 (1H, d, J 7.81 Hz), 8.13 (2H, t, J 2.18 Hz), 7.74 (1H, dd, J 8.94, 6.25 Hz), 7.34 (1H, t, J 9.12 Hz), 6.44 (1H, s), 6.29 (1H, d, J 5.29 Hz), 5.18 (1H, m), 3.83-3.74 (4H, m), 3.15-3.40 (4H, m), 2.56 (3H, s), 2.40-2.50 (2H, m), 1.89 (3H, d, J 6.71 Hz), 1.08 (3H, t, J 7.38 Hz). LCMS (ES+) 462 (M+H)⁺, RT 2.55 minutes (Method 2).

### Example 36

### (S)—N-(1-{7-Fluoro-8-methyl-2-[4-(thiazol-2-yl)piperazin-1-yl]quinolin-3-yl}ethyl)-pyrazolo[1,5-a]pyrimidin-7-amine

Following the procedure described for Example 32, Intermediate 15 (500 mg, 1.48 mmol) and 2-(piperazin-1-yl)thiazole (500 mg, 2.95 mmol) gave the desired product (550 mg, 100%). A portion of this material (59 mg, 0.16 mmol) and 7-chloropyrazolo[1,5-a]-pyrimidine (30 mg, 0.194 mmol) afforded the title compound (20 mg, 28%) as a beige solid. δ_{H} (DMSO-d₆) 8.66 (1H, s), 8.42 (1H, d, J 7.82 Hz), 8.13 (2H, m), 7.75 (1H, dd, J 8.96, 6.24 Hz), 7.34 (1H, t, J 9.13 Hz), 7.27 (1H, d, J 3.61 Hz), 6.95 (1H, d, J 3.61 Hz), 6.43 (1H, s), 6.30 (1H, d, J 5.30 Hz), 5.19 (1H, m), 3.86-3.79 (2H, m), 3.75-3.68 (2H, m), 3.55-3.40 (4H, m), 2.58 (3H, s), 1.90 (3H, d, J 6.70 Hz). LCMS (ES+) 489 (M+H)⁺, RT 3.99 minutes (Method 1).

### Example 37

### (S)—N-(1-{7-Fluoro-8-methyl-2-[4-(pyridin-3-yl)piperazin-1-yl]quinolin-3-yl}ethyl)-pyrazolo[1,5-a]pyrimidin-7-amine

Following the procedure described for Example 32, Intermediate 15 (500 mg, 1.48 mmol) and 1-(pyridin-3-yl)piperazine (482 mg, 2.95 mmol) gave the desired product (540 mg, 100%). A portion of this material (58 mg, 0.16 mmol) and 7-chloropyrazolo[1,5-a]-pyrimidine (30 mg, 0.194 mmol) afforded the title compound (15 mg, 19%) as a beige solid. δ_{H} (DMSO-d₆) 8.66 (1H, s), 8.44 (2H, d, J 11.97 Hz), 8.13-8.06 (3H, m), 7.75 (1H, t, J 7.62 Hz), 7.48 (1H, d, J 8.49 Hz), 7.36-7.27 (2H, m), 6.43 (1H, s), 6.31 (1H, d, J 5.23 Hz), 5.20 (1H, m), 3.64-3.47 (8H, m), 2.59 (3H, s), 1.91 (3H, d, J 6.65 Hz). LCMS (ES+) 483 (M+H)⁺, RT 4.27 minutes (Method 1).

### Example 38

### (S)-(4-{7-Fluoro-8-methyl-3-[1-(pyrazolo[1,5-a]pyrimidin-7-ylamino)ethyl]quinolin-2-yl}piperazin-1-yl)(thien-2-yl)methanone

Following the procedure described for Example 32, Intermediate 15 (150 mg, 0.44 mmol) and (piperazin-1-yl)(thien-2-yl)methanone (108 mg, 0.55 mmol) gave the desired product (175 mg, 100%). A portion of this material (64 mg, 0.16 mmol) and 7-chloro-pyrazolo[1,5-a]pyrimidine (30 mg, 0.194 mmol) afforded the title compound (15 mg, 31%) as an off-white solid. δ_{H} (DMSO-d₆) 8.65 (1H, s), 8.43 (1H, d, J 7.62 Hz), 8.15-8.11 (2H, m), 7.83 (1H, d, J 5.01 Hz), 7.74 (1H, dd, J 8.96, 6.27 Hz), 7.56 (1H, d, J 3.65 Hz), 7.34 (1H, t, J 9.12 Hz), 7.20 (1H, dd, J 5.02, 3.63 Hz), 6.44 (1H, s), 6.30 (1H, d, J 5.30 Hz), 5.19 (1H, m), 4.12-4.04 (2H, m), 4.00-3.93 (2H, m), 3.49-3.30 (4H, m), 2.57 (3H, m), 1.89 (3H, d, J 6.70 Hz). LCMS (ES+) 516 (M+H)⁺, RT 4.09 minutes (Method 1).

### Example 39

### (S)-(4-{7-Fluoro-8-methyl-3-[1-(pyrazolo[1,5-a]pyrimidin-7-ylamino)ethyl]quinolin-2-yl}piperazin-1-yl)(pyridin-4-yl)methanone

Following the procedure described for Example 32, Intermediate 15 (500 mg, 1.48 mmol) and (piperazin-1-yl)(pyridin-4-yl)methanone (778 mg, 2.95 mmol) gave the desired product (582 mg, 100%). A portion of this material (63 mg, 0.16 mmol) and 7-chloropyrazolo[1,5-a]pyrimidine (30 mg, 0.194 mmol) afforded the title compound (27.2 mg, 35%) as a beige solid. δ_{H} (DMSO-d₆) 8.74 (2H, d, J 5.22 Hz), 8.64 (1H, s), 8.42 (1H, d, J 7.75 Hz), 8.14-8.11 (2H, m), 7.74 (1H, dd, J 8.93, 6.26 Hz), 7.53 (2H, d, J 5.23 Hz), 7.34 (1H, t, J 9.11 Hz), 6.43 (1H, s), 6.27 (1H, d, J 5.28 Hz), 5.15 (1H, m), 4.10-3.92 (2H, m), 3.78-3.48 (6H, m), 2.59 (3H, s), 1.88 (3H, d, J 6.67 Hz). LCMS (ES+) 511 (M+H)⁺, RT 3.19 minutes (Method 1).

### Example 40

### (S)-1-(4-{7-Fluoro-3-[1-(pyrazolo[1,5-a]pyrimidin-7-ylamino)ethyl]quinolin-2-yl}-piperazin-1-yl)ethanone

A solution of Intermediate 26 (75 mg, 0.23 mmol), 7-chloropyrazolo[1,5-a]-pyrimidine (40 mg, 0.26 mmol) and DIPEA (100 mg, 0.77 mmol) in n-BuOH (3 mL) was heated at 120° C. for 16 h. The solvent was removed in vacuo and the residue purified by preparative HPLC to afford the title compound (11 mg, 11%) as a cream solid. δ_{H} (CDCl₃) 8.16 (1H, s), 8.14 (1H, d, J 5.2 Hz), 8.02 (1H, s), 7.69 (1H, dd, J 8.8 Hz), 7.52 (1H, dd, J 7.6 Hz), 7.23 (1H, t, J 8.8 Hz), 6.94 (1H, d, J 6.4 Hz), 6.52 (1H, s), 5.94 (1H, d, J 5.2 Hz), 5.10-5.18 (1H, m), 3.90-4.02 (1H, m), 3.80-3.85 (2H, m), 3.66-3.71 (1H, m), 3.32-3.38 (2H, m), 3.20-3.24 (2H, m), 2.20 (3H, s), 1.85 (3H, d, J 6.8 Hz). LCMS (ES+) 434 (M+H)⁺, RT 2.20 minutes (Method 2).

### Example 41

### (S)—N-{1-[7-Fluoro-8-methyl-2-(pyrrolidin-1-yl)quinolin-3-yl]ethyl}pyrazolo[1,5-a]-pyrimidin-7-amine

Intermediate 27 (100 mg, 0.23 mmol) was dissolved in 1,4-dioxane (10 mL) and HCl (4M in 1,4-dioxane, 4 mL) was added. The reaction mixture was stirred at r.t. for 2 h then basified with 15% NaOH solution and extracted with DCM (50 mL). The organic layer was separated, dried (MgSO₄), filtered and concentrated in vacuo to afford a yellow oil (70 mg, 90%). This material (70 mg, 0.21 mmol), 7-chloropyrazolo[1,5-a]pyrimidine (40 mg, 0.26 mmol) and DIPEA (100 mg, 0.78 mmol) in n-BuOH (2.0 mL) were stirred at 110° C. for 18 h. The solvent was removed in vacuo and the residue purified by preparative HPLC to afford the title compound (20 mg, 21%) as a cream solid. δ_{H} (CDCl₃) 8.14 (1H, d, J 5.2 Hz), 8.05 (1H, s), 7.95 (1H, s), 7.39 (1H, dd, J 8.8 Hz), 7.02 (1H, t, J 8.8 Hz), 6.74 (1H, d, J 6.4 Hz), 6.55 (1H, s), 5.84 (1H, d, J 5.2 Hz), 5.22-5.30 (1H, m), 3.72-3.78 (4H, m), 2.59 (3H, s), 2.08-2.18 (4H, m), 1.75 (3H, d, J 6.8 Hz). LCMS (ES+) 391 (M+H)⁺, RT 3.99 minutes (Method 1).

### Example 42

### (S)-1-(4-{7-Fluoro-8-methyl-3-[1-(pyrazolo[1,5-a]pyrimidin-7-ylamino)ethyl]quinolin-2-yl}piperazin-1-yl)ethanone

A solution of Intermediate 28 (70 mg, 0.25 mmol), 7-chloropyrazolo[1,5-a]-pyrimidine (43 mg, 0.27 mmol) and DIPEA (100 mg, 0.77 mmol) in n-BuOH (3 mL) was heated at 120° C. for 16 h. The solvent was removed in vacuo and the residue purified by preparative HPLC to afford the title compound (79.6 mg, 79.6%) as a cream solid. δ_{H} (CDCl₃) 8.15 (1H, s), 8.14 (1H, d, J 5.2 Hz), 8.04 (1H, s), 7.55 (1H, dd, J 8.8 Hz), 7.19 (1H, t, J 8.8 Hz), 6.99 (1H, d, J 6.4 Hz), 6.52 (1H, s), 6.00 (1H, d, J 5.2 Hz), 5.10-5.18 (1H, m), 3.90-4.02 (1H, m), 3.80-3.88 (2H, m), 3.66-3.71 (1 Hon), 3.35-3.41 (2H, m), 3.20-3.24 (2H, m), 2.65 (3H, s), 2.25 (3H, s), 1.89 (3H, d, J 6.8 Hz). LCMS (ES+) 448 (M+H)⁺, RT 2.49 minutes (Method 2).

### Example 43

### (S)-4-{3-[1-(2-Aminopyrazolo[1,5-a][1,3,5]triazin-4-ylamino)ethyl]-7-fluoro-8-methyl-quinolin-2-yl}piperazin-2-one

TFA (1 mL) was added to a stirred solution of Intermediate 21 (1.50 g, 3.73 mmol) and the mixture was allowed to stand at r.t. for 16 h before being concentrated in vacuo. The residue was dissolved in MeOH (10 mL) and purified by SCX column chromatography eluting with MeOH, then 1M NH₃ in MeOH, to afford a clear oil (1.10 g, 97%). To a solution of this oil (100 mg, 0.33 mmol) in DCM (5.0 mL) were added DIPEA (0.1 g, 0.78 mmol) and 4-chloro-2-(methylthio)pyrazolo[1,5-a][1,3,5]triazine (70 mg, 0.34 mmol) and the reaction mixture was stirred for 2 h. The mixture was washed with water (2×15 mL). The organic layer was separated, dried (MgSO₄), and the solvent removed in vacuo to give a yellow oil (135 mg, 88%). This oil was dissolved in DCM (10 mL) and 3-chloroperoxybenzoic acid (148 mg, 0.86 mmol) was added. After stirring overnight, the mixture was washed with saturated NaCO₃ solution (2×50 mL) and water (2×50 mL). The organic layer was separated and dried (MgSO₄), and the solvent was removed in vacuo to give an orange oil (120 mg, 83%). This oil was dissolved in THF (2 mL) and ammonia solution (2.0 mL) and the mixture was heated in a sealed tube at 100° C. for 4 h. The solvent was removed in vacuo and the residue purified by preparative HPLC to afford the title compound (7.6 mg, 14.8%) as a cream solid. δ_{H} (CDCl₃) 8.10 (1H, s), 7.80 (1H, s), 7.51 (1H, dd, J 8.8 Hz), 7.19 (1H, t, J 8.8 Hz), 6.95 (1H, d, J 7.6 Hz), 6.25 (1H, br s), 5.95 (1H, d, J 6.5 Hz), 5.59-5.63 (1H, m), 4.80 (2H, q, J 17.5 Hz), 4.12-4.20 (1H, m), 4.00-4.06 (1H, m), 3.50-3.59 (2H, m), 2.58 (3H, s), 2.20 (2H, br s), 1.55 (3H, d, J 6.8 Hz). LCMS (ES+) 436 (M+H)⁺, RT 2.94 minutes (Method 1).

### Example 44

### (S)-4-{7-Fluoro-8-methyl-3-[1-(pyrazolo[1,5-a]pyrimidin-7-ylamino)ethyl]quinolin-2-yl}piperazin-2-one

TFA (1 mL) was added to a stirred solution of Intermediate 21 (1.50 g, 3.73 mmol) and the mixture was allowed to stand at r.t. for 16 h before being concentrated in vacuo. The residue was dissolved in MeOH (10 mL) and purified by SCX column chromatography eluting with MeOH, then 1M NH₃ in MeOH, to afford a clear oil (1.10 g, 97%). To a solution of this oil (100 mg, 0.33 mmol) in n-BuOH (3 mL) were added 7-chloropyrazolo[1,5-a]pyrimidine (56 mg, 0.37 mmol) and DIPEA (100 mg, 0.78 mmol) and the mixture was heated at 120° C. for 16 h. The solvent was removed in vacuo and the residue purified by preparative HPLC to afford the title compound (13.9 mg, 10%) as a cream solid. δ_{H} (CDCl₃) 8.16 (1H, s), 8.12 (1H, d, J 5.2 Hz), 8.02 (1H, s), 7.52 (1H, dd, J 8.8 Hz), 7.19 (1H, t, J 8.8 Hz), 6.89 (1H, d, J 6.4 Hz), 6.52 (1H, s), 6.12 (1H, s), 5.89 (1H, d, J 5.2 Hz), 5.14-5.23 (1H, m), 4.12 (2H, q, J 17.5 Hz), 3.50-3.59 (4H, m), 2.61 (3H, s), 1.85 (3H, d, J 6.8 Hz). LCMS (ES+) 420 (M+H)⁺, RT 2.16 minutes (Method 2).

### Example 45

### (S)-4-{7-Fluoro-3-[1-(pyrazolo[1,5-a]pyrimidin-7-ylamino)ethyl]quinolin-2-yl}piperazin-2-one

A solution of Intermediate 29 (55 mg, 0.19 mmol), 7-chloropyrazolo[1,5-a]-pyrimidine (33.8 mg, 0.22 mmol) and DIPEA (28.0 mg, 0.22 mmol) in n-BuOH (3 mL) was heated at 120° C. for 16 h. The solvent was removed in vacuo and the residue purified by preparative HPLC to afford the title compound (54.8 mg, 71%) as a cream solid. δ_{H} (CDCl₃) 8.21 (1H, s), 8.13 (1H, d, J 5.2 Hz), 8.02 (1H, s), 7.70 (1H, dd, J 8.8 Hz), 7.55 (1H, dd, J 8.8 Hz), 7.19 (1H, t, J 8.8 Hz), 7.02 (1H, br s), 6.89 (1H, d, J 6.4 Hz), 6.52 (1H, s), 5.89 (1H, d, J 5.2 Hz), 5.14-5.20 (1H, m), 4.12 (2H, q, J 17.5 Hz), 3.53-3.68 (4H, m), 1.85 (3H, d, J 6.8 Hz). LCMS (ES+) 406 (M+H)⁺, RT 2.10 minutes (Method 1).

### Example 46

### (S)-4-{5,7-Difluoro-3-[1-(pyrazolo[1,5-a]pyrimidin-7-ylamino)ethyl]quinolin-2-yl}-piperazin-2-one

A solution of Intermediate 30 (61.2 mg, 0.20 mmol), 7-chloropyrazolo[1,5-a]-pyrimidine (33.8 mg, 0.2 mmol) and DIPEA (28.5 mg, 0.22 mmol) in n-BuOH (3 mL) was heated at 120° C. for 16 h. The solvent was removed in vacuo and the residue purified by preparative HPLC to afford the title compound (14.9 mg, 18%) as a cream solid. δ_{H} (CDCl₃) 8.45 (1H, s), 8.13 (1H, d, J 5.2 Hz), 8.02 (1H, s), 7.35 (1H, dd, J 8.8 Hz), 6.90 (1H, t, J 8.8 Hz), 6.88 (1H, d, J 6.4 Hz), 6.55 (1H, br s), 6.48 (1H, s), 5.89 (1H, d, J 5.2 Hz), 5.12-5.18 (1H, m), 4.09 (2H, q, J 17.5 Hz), 3.52-3.68 (4H, m), 1.88 (3H, d, J 6.8 Hz). LCMS (ES+) 424 (M+H)⁺, RT 3.07 minutes (Method 1).

### Example 47

### (S)-1-(4-{8-Chloro-3-[1-(pyrazolo[1,5-a]pyrimidin-7-ylamino)ethyl]quinolin-2-yl}-piperazin-1-yl)ethanone

To a solution of Intermediate 31 (86.5 mg, 0.2 mmol) in 1,4-dioxane (1 mL) was added HCl (2.25 mL; 4M in 1,4-dioxane). The mixture was stirred at r.t. for 1 h, then the excess solvent was removed in vacuo. The resulting material was dissolved in n-BuOH (2.5 mL) and DIPEA (0.78 mg, 0.6 mmol) and 7-chloropyrazolo[1,5-a]pyrimidine (38 mg, 0.25 mmol) were added. The mixture was heated at 120° C. for 16 h and the solvent was removed in vacuo. The residue was purified by preparative HPLC to afford the title compound (22.8 mg, 17%) as an off-white solid. δ_{H} (CDCl₃) 8.19 (1H, s), 8.13 (1H, d, J 5.2 Hz), 8.02 (1H, s), 7.73 (1H, d, J 8.8 Hz), 7.62 (1H, d, J 8.8 Hz), 7.32 (1H, t, J 8.8 Hz), 6.89 (1H, d, J 6.8 Hz), 6.52 (1H, s), 5.92 (1H, d, J 5.2 Hz), 5.12-5.20 (1H, m), 3.92-3.98 (1H, m), 3.84-3.92 (2H, m), 3.70-3.76 (1H, m), 3.48-3.54 (2H, m), 3.32-3.36 (2H, m), 2.21 (3H, s), 1.87 (3H, d, J 6.8 Hz). LCMS (ES+) 450/452 (M+H)⁺, RT 2.35 minutes (Method 2).

### Example 48

### (S)-1-(4-{3-[1-(2-Aminopyrazolo[1,5-a][1,3,5]triazin-4-ylamino)ethyl]-8-chloroquinolin-2-yl}piperazin-1-yl)ethanone

To a solution of Intermediate 31 (193 mg, 0.45 mmol) in 1,4-dioxane (2.25 mL) was added HCl (5.1 mL; 4M in 1,4-dioxane). The mixture was stirred at r.t. for 1 h, then the excess solvent was removed in vacuo. To a solution of the resulting material (332 mg, 1.0 mmol) in DCM (5.0 mL) were added DIPEA (0.2 g, 1.56 mmol) and 4-chloro-2-(methylthio)pyrazolo[1,5-a][1,3,5]triazine (220 mg, 1.1 mmol) and the reaction mixture was stirred at r.t. for 2 h. The mixture was diluted with DCM (30 mL) and washed with water (2×15 mL). The organic layer was separated and dried (MgSO₄), and the solvent was removed in vacuo to give a yellow solid (300 mg, 67%). This solid was dissolved in DCM (50 mL) and 3-chloroperoxybenzoic acid (480 mg, 1.3 mmol) was added. After stirring overnight, the mixture was washed with saturated NaCO₃ solution (2×50 mL) and water (2×50 mL). The organic layer was separated and dried (MgSO₄), and the solvent was removed in vacuo to give an orange oil (294 mg, 92%). A portion of this oil (100 mg, 0.19 mmol) was dissolved in methanol (2.0 mL) and ammonia solution (2.0 mL) and the solution was heated in a sealed tube at 100° C. for 16 h. The solvent was removed in vacuo and the residue purified by preparative HPLC to afford the title compound (27.9 mg, 32%) as a cream solid. δ_{H} (CDCl₃) 8.10 (1H, s), 7.81 (1H, d, J 2.4 Hz), 7.74 (1H, d, J 8.8 Hz), 7.62 (1H, d, J 8.8 Hz), 7.32 (1H, t, J 8.8 Hz), 7.03 (1H, d, J 6.8 Hz), 5.95 (1H, d, J 2.4 Hz), 5.61-5.68 (1H, m), 4.92 (2H, s), 3.92-3.98 (1H, m), 3.80-3.88 (2H, m), 3.62-3.70 (3H, m), 3.32-3.38 (1H, m), 3.20-3.25 (1H, m), 2.21 (3H, s), 1.67 (3H, d, J 6.8 Hz). LCMS (ES+) 466/468 (M+H)⁺, RT 2.44 minutes (Method 2).

### Example 49

### (S)-1-(4-{8-Chloro-3-[1-(2-methoxypyrazolo[1,5-a][1,3,5]triazin-4-ylamino)ethyl]-quinolin-2-yl}piperazin-1-yl)ethanone

The title compound (22.7 mg, 23%) was obtained as a by-product from the procedure described in Example 48 and was isolated as a cream solid. δ_{H} (CDCl₃) 8.10 (1H, s), 7.88 (1H, d, J 2.4 Hz), 7.74 (1H, d, J 8.8 Hz), 7.62 (1H, d, J 8.8 Hz), 7.32 (2H, t, J 8.8 Hz), 6.20 (1H, d, J 2.4 Hz), 5.72-5.78 (1H, m), 3.96 (3H, s), 3.91-3.96 (1H, m), 3.80-3.88 (2H, m), 3.62-3.68 (3H, m), 3.34-3.39 (1H, m), 3.22-3.28 (1H, m), 2.19 (3H, s), 1.69 (3H, d, J 6.8 Hz). LCMS (ES+) 481/483 (M+H)⁺, RT 3.46 minutes (Method 2).

### Example 50

### (S)-1-(4-{8-Chloro-3-[1-(pyrazolo[1,5-a][1,3,5]triazin-4-ylamino)ethyl]quinolin-2-yl}-piperazin-1-yl)ethanone

To a solution of Intermediate 31 (193 mg, 0.45 mmol) in 1,4-dioxane (2.25 mL) was added HCl (4M in 1,4-dioxane; 5.1 mL). The mixture was stirred at r.t. for 1 h. The excess solvent was removed in vacuo. To a solution of the resulting material (332 mg, 1.0 mmol) in DCM (5.0 mL) were added DIPEA (0.2 g, 1.56 mmol) and 4-chloro-2-(methylthio)pyrazolo[1,5-a][1,3,5]triazine (220 mg, 1.1 mmol) and the reaction mixture was stirred at r.t. for 2 h. The mixture was diluted with DCM (20 mL) and washed with water (2×15 mL). The organic layer was separated and dried (MgSO₄), and the solvent was removed in vacuo to give a yellow solid (300 mg, 67%). This solid was dissolved in DCM (50 mL) and 3-chloroperoxybenzoic acid (480 mg, 1.3 mmol) was added. After stirring overnight, the mixture was washed with saturated NaCO₃ solution (2×50 mL) and water (2×50 mL). The organic layer was separated and dried (MgSO₄), and the solvent was removed in vacuo to give an orange oil (294 mg, 92%). This oil (100 mg, 0.19 mmol) was dissolved in ethanol (5.0 mL) and treated with NaBH₄ (20 mg, 0.53 mmol). The mixture was stirred at r.t. for 2 h before being quenched with water (25 mL) and extracted with DCM (2×25 mL). The organic layers were combined, dried (MgSO₄) and the solvent removed in vacuo. The residue was purified by preparative HPLC to afford the title compound (9.3 mg, 11%) as a cream solid. δ_{H} (CDCl₃) 9.48 (1H, s), 8.58 (1H, s), 8.25 (1H, d, J 2.4 Hz), 8.19 (1H, s), 7.84 (2H, d, J 8.8 Hz), 7.45 (1H, t, J 8.8 Hz), 6.54 (1H, d, J 2.4 Hz), 5.88-5.92 (1H, m), 3.78-3.82 (1H, m), 3.74-3.78 (2H, m), 3.58-3.64 (3H, m), 3.22-3.28 (1H, m), 3.18-3.22 (1H, m), 2.10 (3H, s), 1.67 (3H, d, J 6.8 Hz). LCMS (ES+) 451/453 (M+H)⁺, RT 3.62 minutes (Method 1).

### Example 51

### (S)-1-(4-{5,7-Difluoro-3-[1-(pyrazolo[1,5-a]pyrimidin-7-ylamino)ethyl]quinolin-2-yl}-piperazin-1-yl)ethanone

A solution of Intermediate 32 (70 mg, 0.21 mmol), 7-chloropyrazolo[1,5-a]-pyrimidine (37 mg, 0.24 mmol) and DIPEA (50 mg, 0.4 mmol) in n-BuOH (3 mL) was heated at 120° C. for 16 h. The solvent was removed in vacuo and the residue purified by preparative HPLC to afford the title compound (84.8 mg, 89%) as a cream solid. δ_{H} (CDCl₃) 8.42 (1H, s), 8.18 (1H, d, J 5.2 Hz), 8.02 (1H, d, J 2.4 Hz), 7.33 (1H, d, J 8.8 Hz), 7.12 (1H, d, J 8.8 Hz), 6.95 (1H, t, J 8.8 Hz), 6.54 (1H, d, J 2.4 Hz), 6.00 (1H, d, J 5.2 Hz), 5.14-5.20 (1H, m), 3.93-3.99 (1H, m), 3.80-3.88 (2H, m), 3.68-3.74 (1H, m), 3.39-3.43 (2H, m), 3.20-3.30 (2H, m), 2.20 (3H, s), 1.87 (3H, d, J 6.8 Hz). LCMS (ES+) 452 (M+H)⁺, RT 2.96 minutes (Method 1).

### Example 52

### 1-(4-{7-Fluoro-8-methyl-3-[(pyrazolo[1,5-a][1,3,5]triazin-4-ylamino)methyl]quinolin-2-yl}piperazin-1-yl)ethanone

A solution of Intermediate 34 (50 mg, 0.16 mmol), 4-chloro-2-(methylthio)-pyrazolo[1,5-a][1,3,5]triazine (35 mg, 0.17 mmol) and DIPEA (40 mg, 0.31 mmol) in DCM (3.0 mL) was stirred for 2 h at r.t. The mixture was diluted with DCM (20 mL) and washed with water (2×15 mL). The organic layer was separated, dried (MgSO₄) and the solvent removed in vacuo to give a yellow solid (64 mg, 84%). This solid was dissolved in DCM (5.0 mL) and 3-chloroperoxybenzoic acid (45 mg, 0.26 mmol) was added. After stirring overnight, the mixture was washed with saturated NaCO₃ solution (2×50 mL) and water (2×50 mL). The organic layer was separated and dried (MgSO₄), and the solvent removed in vacuo. The residue was dissolved in ethanol (5 mL) and treated with NaBH₄ (20 mg, 0.53 mmol). The mixture was stirred for 2 h before being quenched with water (25 mL) and extracted with DCM (2×25 mL). The organic layers were combined, dried (MgSO₄) and the solvent removed in vacuo. Purification of the residue by preparative HPLC afforded the title compound (2.5 mg, 5%) as a cream solid. δ_{H} (CDCl₃) 8.24 (1H, s), 8.00 (1H, s), 7.98 (1H, d, J 2.4 Hz), 7.86 (1H, br t, J 8.8 Hz), 7.54 (1H, dd, J 8.8 Hz), 7.19 (1H, t, J 8.8 Hz), 6.47 (1H, d, J 2.4 Hz), 5.06 (2H, d, J 5.6 Hz), 3.88-3.92 (2H, m), 3.76-3.80 (2H, m), 3.41-3.45 (2H, m), 3.31-3.35 (2H, m), 2.61 (3H, s), 2.19 (3H, s). LCMS (ES+) 435 (M+H)⁺, RT 3.06 minutes (Method 1).

### Example 53

### (S)-1-(4-{5,7-Difluoro-3-[1-(pyrazolo[1,5-a][1,3,5]triazin-4-ylamino)ethyl]quinolin-2-yl}piperazin-1-yl)ethanone

A solution of Intermediate 32 (70 mg, 0.21 mmol), 4-chloro-2-(methylthio)-pyrazolo[1,5-a][1,3,5]triazine (48 mg, 0.24 mmol) and DIPEA (50 mg, 0.4 mmol) in DCM (5 mL) was stirred for 2 h at r.t. The mixture was diluted with DCM (20 mL) and washed with water (2×15 mL). The organic layer was separated and dried (MgSO₄), and the solvent was removed in vacuo to give a pale orange solid (90 mg, 86%). This solid was dissolved in DCM (5.0 mL) and 3-chloroperoxybenzoic acid (62 mg, 0.36 mmol) was added. After stirring overnight, the mixture was washed with saturated NaCO₃ solution (2×50 mL) and water (2×50 mL). The organic layer was separated, dried (MgSO₄) and the solvent removed in vacuo. The residue was dissolved in ethanol (5 mL) and treated with NaBH₄ (20 mg, 0.53 mmol). After stirring for 2 h, the mixture was quenched with water (25 mL) and extracted with DCM (2×25 mL). The organic layers were combined, dried (MgSO₄) and the solvent removed in vacuo. Purification of the residue by preparative HPLC afforded the title compound (8.2 mg, 10%) as a cream solid. δ_{H} (CDCl₃) 8.31 (1H, s), 8.18 (1H, s), 8.02 (1H, d, J 2.4 Hz), 7.34 (1H, d, J 8.8 Hz), 7.20 (1H, d, J 8.8 Hz), 6.93 (1H, t, J 8.8 Hz), 6.49 (1H, d, J 2.4 Hz), 5.76-5.84 (1H, m), 3.84-3.92 (2H, m), 3.68-3.76 (2H, m), 3.54-3.62 (2H, m), 3.18-3.28 (2H, m), 2.17 (3H, s), 1.73 (3H, d, J 6.8 Hz). LCMS (ES+) 453 (M+H)⁺, RT 3.17 minutes (Method 2).

### Example 54

### 1-(4-{7-Fluoro-8-methyl-3-[(pyrazolo[1,5-a]pyrimidin-7-ylamino)methyl]quinolin-2-yl}-piperazin-1-yl)ethanone

A solution of Intermediate 34 (50 mg, 0.16 mmol), 7-chloropyrazolo[1,5-a]-pyrimidine (30 mg, 0.19 mmol) and DIPEA (40 mg, 0.31 mmol) in n-BuOH (3 mL) was heated at 120° C. for 16 h. The solvent was removed in vacuo and the residue purified by preparative HPLC to afford the title compound (30 mg, 44%) as a cream solid. δ_{H} (CDCl₃) 8.20 (1H, d, J 2.4 Hz), 8.02 (1H, d, J 2.4 Hz), 8.00 (1H, s), 7.48-7.54 (1H, m), 7.44 (1H, br s), 7.18 (1H, t, J 8.8 Hz), 6.54 (1H, d, J 2.4 Hz), 5.90 (1H, d, J 2.4 Hz), 4.78 (2H, d, J 5.6 Hz), 3.88-3.92 (2H, m), 3.76-3.80 (2H, m), 3.44-3.48 (2H, m), 3.28-3.32 (2H, m), 2.61 (3H, s), 2.05 (3H, s). LCMS (ES+) 434 (M+H)⁺, RT 3.39 minutes (Method 1).

### Example 55

### (S)-1-(4-{3-[1-(Pyrazolo[1,5-a]pyrimidin-7-ylamino)ethyl]quinolin-2-yl}piperazin-1-yl)-ethanone

A solution of Intermediate 35 (70 mg, 0.23 mmol), 7-chloropyrazolo[1,5-a]-pyrimidine (39 mg, 0.25 mmol) and DIPEA (60 mg, 0.46 mmol) in n-BuOH (3 mL) was heated at 120° C. for 16 h. The solvent was removed in vacuo and the residue purified by preparative HPLC to afford the title compound (15.2 mg, 17%) as a cream solid. δ_{H} (CDCl₃) 8.18 (1H, s), 8.14 (1H, d, J 5.2 Hz), 8.02 (1H, d, J 2.4 Hz), 7.89 (1H, d, J 8.8 Hz), 7.72 (1H, d, J 8.8 Hz), 7.66 (1H, t, J 8.8 Hz), 7.63 (1H, t, J 8.8 Hz), 6.94 (1H, d, J 6.4 Hz), 6.51 (1H, d, J 2.4 Hz), 5.94 (1H, d, J 5.2 Hz), 5.17-5.23 (1H, m), 3.94-3.98 (1H, m), 3.80-3.88 (2H, m), 3.65-3.73 (1H, m), 3.30-3.41 (2H, m), 3.22-3.26 (2H, m), 2.20 (3H, s), 1.86 (3H, d, J 6.8 Hz). LCMS (ES+) 416 (M+H)⁺, RT 2.60 minutes (Method 1).

### Example 56

### Methyl 4-{7-fluoro-8-methyl-3-[(1S)-1-(pyrazolo[1,5-a]pyrimidin-7-ylamino)ethyl]-quinolin-2-yl}piperazine-1-carboxylate

A mixture of Intermediate 15 (501 mg, 1.48 mmol), methyl piperazine-1-carboxylate (981 mg, 6.80 mmol) and DIPEA (1.29 mL, 7.39 mmol) in NMP (3 mL) was heated under microwave irradiation at 130° C. for 4.5 h. After cooling, the mixture was dissolved in a 1:1 mixture of EtOAc and Et₂O (250 mL) and washed with saturated brine (3×50 mL). The organic layer was separated, dried (MgSO₄), filtered and concentrated in vacuo. Purification by column chromatography (SiO₂, 10% EtOAc in DCM) gave a pale yellow oil (400 mg, 60%). LCMS (ES+) 447 (M+H)⁺. To this oil (400 mg, 0.896 mmol) dissolved in DCM (23 mL) was added TFA (4.1 mL). The reaction mixture was stirred at r.t. for 1.5 h. The excess solvent was removed in vacuo. The oil obtained was basified with 0.2M NaOH (40 mL) and extracted with EtOAc (3×80 mL). The combined organic layers were dried (MgSO₄), filtered and concentrated in vacuo. Purification by column chromatography (SiO₂, 95:4:1 DCM/MeOH/NH₃ solution in MeOH) gave a colourless gum (278 mg, 90%). LCMS (ES+) 347 (M+H)⁺. A portion of this material (55.6 mg, 0.161 mmol), 7-chloropyrazolo[1,5-a]pyrimidine (37 mg, 0.241 mmol), DIPEA (0.084 mL, 0.482 mmol) and n-BuOH (1 mL) were combined and heated under microwave irradiation at 130° C. for 1 h. Purification by preparative HPLC gave the title compound (43.7 mg, 59%) as a yellow solid. δ_{H} (DMSO-d₆) 8.64 (s, 1H), 8.41 (d, J 7.84 Hz, 1H), 8.11-8.14 (m, 2H), 7.74 (dd, J 8.95, 6.27 Hz, 1H), 7.34 (t, J 9.12 Hz, 1H), 6.44 (d, J 2.27 Hz, 1H), 6.27 (d, J 5.30 Hz, 1H), 5.16 (t, J 7.20 Hz, 1H), 3.75-3.83 (m, 2H), 3.70 (s, 3H), 3.65-3.70 (m, 2H), 3.30-3.35 (m, 2H), 3.22-3.30 (m, 2H), 2.11 (s, 3H), 1.88 (d, J 6.71 Hz, 31-1). LCMS (ES+) 464 (M+H)⁺, RT 3.71 minutes (Method 1).

### Example 57

### 4-{7-Fluoro-8-methyl-3-[(1S)-1-(pyrazolo[1,5-a]pyrimidin-7-ylamino)ethyl]quinolin-2-yl}-N-methylpiperazine-1-carboxamide

A solution of Intermediate 15 (500 mg, 1.476 mmol), 1-[(methylamino)carbonyl]-piperazine (677 mg, 4.73 mmol) and DIPEA (1.54 mL, 8.85 mmol) in NMP (3.5 mL) was heated under microwave irradiation at 150° C. for 3.5 h. After cooling, the mixture was dissolved in a 1:1 mixture of EtOAc and Et₂O (250 mL) and washed with saturated brine (3×50 mL). The organic layer was separated, dried (MgSO₄), filtered and concentrated in vacuo. Purification by column chromatography (SiO₂, 2% EtOAc in DCM) gave a pale yellow solid (214 mg, 33%). LCMS (ES+) 446 (M+H)⁺. To this solid (214 mg, 0.48 mmol) dissolved in DCM (12 mL) was added TFA (2.2 mL). The reaction mixture was stirred at r.t. for 1.5 h and the excess solvent was removed in vacuo. The oil obtained was basified with 0.5M NaOH solution (20 mL) and extracted with EtOAc (3×50 mL). The combined organic layers were dried (MgSO₄), filtered and concentrated in vacuo to give a pale yellow glass (179 mg, 100%). LCMS (ES+) 346 (M+H)⁺. A portion of this material (44.7 mg, 0.129 mmol), 7-chloropyrazolo[1,5-a]pyrimidine (29.8 mg, 0.194 mmol), DIPEA (0.068 mL, 0.388 mmol) and n-BuOH (1 mL) were combined and heated under microwave irradiation at 130° C. for 1 h. Purification by preparative HPLC gave the title compound (5.5 mg, 9%) as a yellow solid. δ_{H} (DMSO-d₆) 8.64 (s, 1H), 8.41 (d, J 7.85 Hz, 1H), 8.11-8.14 (m, 2H), 7.74 (dd, J 8.96, 6.27 Hz, 1H), 7.33 (t, J 9.12 Hz, 1H), 6.60 (d, J 4.79 Hz, 1H), 6.44 (d, J 2.28 Hz, 1H), 6.29 (d, J 5.31 Hz, 1H), 5.16 (t, J 7.21 Hz, 1H), 3.56-3.72 (m, 4H), 3.18-3.34 (m, 4H), 2.65 (d, J 4.23 Hz, 3H), 2.56 (s, 3H), 1.87 (d, J 6.72 Hz, 3H). LCMS (ES+) 463 (M+H)⁺, RT 3.08 minutes (Method 1).

### Example 58

### 4-{7-Fluoro-8-methyl-3-[(1S)-1-(pyrazolo[1,5-a]pyrimidin-7-ylamino)ethyl]quinolin-2-yl}piperazine-1-carboxamide

Intermediate 36 (50 mg, 0.151 mmol), 7-chloropyrazolo[1,5-a]pyrimidine (34.7 mg, 0.226 mmol), DIPEA (0.079 mL, 0.453 mmol) and n-BuOH (1 mL) were combined and heated under microwave irradiation at 140° C. for 1 h. After cooling, the mixture was dissolved in EtOAc (100 mL) and washed with saturated brine (3×20 mL). The organic layer was separated, dried (MgSO₄), filtered and concentrated in vacuo. Purification by preparative HPLC gave the title compound (7 mg, 10%) as a yellow solid. δ_{H} (DMSO-d₆) 8.24 (d, J 4.99 Hz, 1H), 8.14 (s, 1H), 8.10 (d, J 2.33 Hz, 1H), 7.70 (dd, J 8.89, 6.22 Hz, 1H), 7.25 (t, J 9.13 Hz, 1H), 6.58 (d, J 7.55 Hz, 1H), 6.51 (d, J 2.33 Hz, 1H), 6.40 (d, J 5.03 Hz, 1H), 5.46 (s, 2H), 5.05 (t, J 6.89 Hz, 1H), 3.95-4.02 (m, 2H), 3.81-3.88 (m, 2H), 3.67-3.75 (m, 2H), 3.27-3.32 (m, 2H), 2.49 (d, J 2.33 Hz, 3H), 1.29 (d, J 6.59 Hz, 3H). LCMS (ES+) 449 (M+H)⁺, RT 2.59 minutes (Method 2).

### Example 59

### (R)-4-{8-Chloro-3-[1-(pyrazolo[1,5-a]pyrimidin-7-ylamino)ethyl]quinolin-2-yl}-piperazin-2-one

TFA (2 mL) was added to a solution of Intermediate 41 (232 mg, 0.57 mmol) in DCM (5 mL) and the mixture was allowed to stand at r.t. overnight before being concentrated in vacuo. The residue was dissolved in DCM (20 mL) and washed with 2M NaOH solution (2×20 mL) and water (20 mL). The organic layer was separated, dried (MgSO₄), filtered and concentrated in vacuo. To the resulting material dissolved in n-BuOH (2.0 mL) and DIPEA (0.30 mL, 1.7 mmol) was added 7-chloropyrazolo[1,5-a]-pyrimidine (115 mg, 0.75 mmol) and the mixture was heated to 120° C. overnight in a sealed tube. The mixture was cooled and the solvent removed in vacuo. The residue was redissolved in DCM (30 mL) and washed with water (20 mL) and brine (20 mL). The organic layer was separated, dried (MgSO₄) and filtered, and the solvent was removed in vacuo. Purification by preparative HPLC gave the title compound (132 mg, 54%) as a white solid. δ_{H} (CDCl₃) 8.21 (1H, s), 8.12 (1H, d, J 5.11 Hz), 8.02 (1H, d, J 2.32 Hz), 7.75 (1H, dd, J 7.52, 1.31 Hz), 7.62 (1H, dd, J 8.16, 1.31 Hz), 7.33 (1H, t, J 7.83 Hz), 6.84 (1H, d, J 6.41 Hz), 6.52 (1H, d, J 2.32 Hz), 6.45-6.40 (1H, m), 5.85 (1H, d, J 5.15 Hz), 5.20-5.10 (1H, m), 4.20 (1H, d, J 17.26 Hz), 4.11 (1H, d, J 17.26 Hz), 3.80-3.61 (4H, m), 1.87 (3H, d, J 6.71 Hz). LCMS (ES+) 422/424 (M+H)⁺, RT 2.22 minutes (Method 2).

### Example 60

### 1-{7-Fluoro-8-methyl-3-[(S)-1-(pyrazolo[1,5-a]pyrimidin-7-ylamino)ethyl]quinolin-2-yl}pyrrolidin-2-one

A mixture of Intermediate 42 (100 mg, 0.28 mmol), 2-pyrrolidinone (35 mg, 0.41 mmol), copper(I) iodide (6 mg, 0.032 mmol), potassium phosphate (130 mg, 0.613 mmol) and N,N′-dimethylcyclohexane-1,2-diamine (5 μL, 0.03 mmol) in toluene (1 mL) was degassed and stirred under nitrogen at 110° C. for 4 h. The reaction mixture was allowed to cool to r.t., diluted with EtOAc (50 mL) and washed with brine (20 mL). The organic layer was separated, dried (MgSO₄) and concentrated in vacuo. Purification by preparative HPLC gave the title compound (22.6 mg, 20%) as a white solid. δ_{H} (DMSO-d₆) 8.48 (s, 1H), 8.28 (m, 1H), 8.12 (m, 2H), 7.87 (dd, J 9.1, 6.3 Hz, 1H), 7.46 (t, J 9.3 Hz, 1H), 6.46 (d, J 2.3 Hz, 1H), 6.25 (d, J 5.3 Hz, 1H), 5.24 (m, 1H), 4.15 (m, 1H), 4.05 (m, 1H), 2.60 (m, 5H), 2.15 (m, 2H), 1.70 (d, J 6.8 Hz, 3H). LCMS (ES+) 405 (M+H)⁺.

### Example 61

### N-{(S)-1-[2-(5,6-Dihydro-8H-[1,2,4]triazolo[4,3-d]pyrazin-7-yl)-7-fluoro-8-methyl-quinolin-3-yl]ethyl}pyrazolo[1,5-a]pyrimidin-7-ylamine

A mixture of Intermediate 42 (100 mg, 0.28 mmol), 5,6,7,8-tetrahydro-[1,2,4]-triazolo[4,3-a]pyrazine (54 mg, 0.425 mmol), potassium tert-butoxide (63 mg, 0.56 mmol), Q-Phos (20 mg, 0.028 mmol) and tris(dibenzylideneacetone)dipalladium(0) (12.8 mg, 0.014 mmol) in toluene (1 mL) was degassed and heated at reflux under nitrogen for 24 h. After cooling, the mixture was diluted with DCM (50 mL) and washed with brine (20 mL). The organic layer was separated, dried (MgSO₄) and concentrated in vacuo. Purification by preparative HPLC gave the title compound (9.3 mg, 7%) as a pale yellow solid. δ_{H} (DMSO-d₆) 8.64 (s, 1H), 8.53 (s, 1H), 8.29 (d, J 7.3 Hz, 1H), 8.08 (m, 2H), 7.75 (dd, J 8.8, 6.3 Hz, 1H), 7.33 (t, J 9.3 Hz, 1H), 6.42 (d, J 2.3 Hz, 1H), 6.12 (d, J 5.3 Hz, 1H), 5.20 (m, 1H), 4.86 (m, 1H), 4.69 (m, 1H), 4.37 (m, 1H), 4.18 (m, 1H), 3.75 (m, 2H), 2.45 (d, J 2.0 Hz, 3H), 1.86 (d, J 6.8 Hz, 3H). LCMS (ES+) 444 (M+H)⁺.

### Example 62

### N—{(S)-1-[7-Fluoro-8-methyl-2-(3-methyl-5,6-dihydro-8H-[1,2,4]triazolo[4,3-a]pyrazin-7-yl)quinolin-3-yl]ethyl}pyrazolo[1,5-a]pyrimidin-7-ylamine

A mixture of Intermediate 42 (100 mg, 0.28 mmol), 3-methyl-5,6,7,8-tetrahydro-[1,2,4]triazolo[4,3-a]pyrazine (54 mg, 0.425 mmol), potassium tert-butoxide (63 mg, 0.56 mmol), Q-Phos (20 mg, 0.028 mmol) and tris(dibenzylideneacetone)dipalladium(0) (12.8 mg, 0.014 mmol) in toluene (1 mL) was degassed and heated at reflux under nitrogen for 24 h. After cooling, the mixture was diluted with DCM (50 mL) and washed with brine (20 mL). The organic layer was separated, dried (MgSO₄) and concentrated in vacuo. Purification by preparative HPLC gave the title compound (19.2 mg, 15%) as an off-white solid. δ_{H} (DMSO-d₆) 8.63 (s, 1H), 8.31 (m, 1H), 8.09 (d, J 2.3 Hz, 1H), 8.04 (d, J 5.3 Hz, 1H), 7.74 (dd, J 9.0, 6.4 Hz, 1H), 7.32 (t, J 9.0 Hz, 1H), 6.41 (d, J 2.3 Hz, 1H), 6.08 (d, J 5.3 Hz, 1H), 5.18 (m, 1H), 4.79 (m, 1H), 4.62 (m, 1H), 4.23 (m, 1H), 4.03 (m, 1H), 3.77 (m, 2H), 2.48 (s, 3H), 2.34 (s, 3H), 1.87 (d, J 6.8 Hz, 31-1). LCMS (ES+) 458 (M+H)⁺.

### Example 63

### 3-{7-Fluoro-8-methyl-3-[(S)-1-(pyrazolo[1,5-a]pyrimidin-7-ylamino)ethyl]quinolin-2-yl}oxazolidin-2-one

A mixture of Intermediate 42 (50 mg, 0.14 mmol), 2-oxazolidinone (18 mg, 0.21 mmol), copper(I) oxide (3 mg, 0.016 mmol), N,N′-dimethylcyclohexane-1,2-diamine (3.0 μL, 0.019 mmol) and potassium phosphate (60 mg, 0.28 mmol) in toluene (1 mL) was degassed and heated under nitrogen at 90° C. overnight. After cooling, the reaction mixture was partitioned between EtOAc (50 mL) and brine (20 mL). The organic layer was separated, dried (MgSO₄) and the solvent removed in vacuo. Purification by preparative HPLC gave the title compound (9.9 mg, 17%) as a pale yellow solid. δ_{H} (DMSO-d₆) 8.55 (s, 1H), 8.32 (d, J 7.8 Hz, 1H), 8.15 (d, J 2.3 Hz, 1H), 8.10 (d, J 1.3 Hz, 1H), 7.87 (dd, J 9.1, 6.3 Hz, 1H), 7.47 (t, J 9.1 Hz, 1H), 6.46 (d, J 2.0 Hz, 1H), 6.19 (d, J 5.1 Hz, 1H), 5.31 (t, J 7.1 Hz, 1H), 4.64 (m, 2H), 4.47 (q, J 8.3 Hz, 1H), 4.36 (m, 1H), 2.57 (d, J 2.3 Hz, 3H), 1.75 (d, J 6.8 Hz, 3H). LCMS (ES+) 407 (M+H)⁺.

1. A compound of formula (I) or an N-oxide thereof, or a pharmaceutically acceptable salt or solvate thereof: 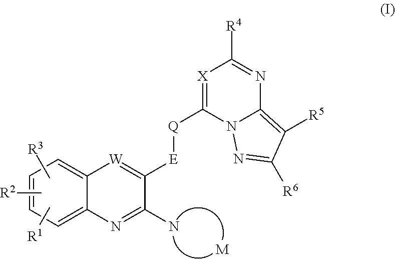
wherein
X represents N or C—R⁷;
E represents an optionally substituted straight or branched C₁₋₄ alkylene chain;
Q represents oxygen, sulfur, N—R⁸ or a covalent bond;
M represents the residue of an optionally substituted saturated five-, six- or seven-membered monocyclic ring containing one nitrogen atom and 0, 1, 2 or 3 additional heteroatoms independently selected from N, O and S, but containing no more than one O or S atom, which ring may be optionally fused to an optionally substituted heteroaromatic ring;
W represents C—R⁹ or N;
R¹, R² and R³ independently represent hydrogen, halogen, cyano, nitro, C₁₋₆ alkyl, trifluoromethyl, aryl(C₁₋₆)alkyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkyl-amino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl;
R⁴, R⁵, R⁶ and R⁷ independently represent C₁₋₆ alkyl, aryl, aryl(C₁₋₆)alkyl, heteroaryl or heteroaryl(C₁₋₆)alkyl, any of which groups may be optionally substituted by one or more substituents; or hydrogen, halogen, trifluoromethyl, —OR^{a}, —SR^{a}, —SOR^{a}, —SO₂R^{a}, —NR^{b}R^{c}, —NR^{c}OR^{d}, —NR^{c}CO₂R^{d}, —NR^{c}SO₂R^{e}, —COR^{d}, —CO₂R^{d}, —CONR^{b}R^{c} or —SO₂NR^{b}R^{c};
R⁸ represents hydrogen or C₁₋₆ alkyl;
R⁹ represents hydrogen, halogen, C₁₋₆ alkyl or C₁₋₆ alkoxy;
R^{a} represents C₁₋₆ alkyl, difluoromethyl or trifluoromethyl;
R^{b} represents hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl-(C₁₋₆)alkyl, heteroaryl or heteroaryl(C₁₋₆)alkyl, any of which groups may be optionally substituted by one or more substituents;
R^{c} represents hydrogen, C₁₋₆ alkyl or C₃₋₇ cycloalkyl;
R^{d} represents hydrogen or C₁₋₆ alkyl; and
R^{e} represents C₁₋₆ alkyl.

2. A compound as claimed in claim 1 represented by formula (IIA) or an N-oxide thereof, or a pharmaceutically acceptable salt or solvate thereof: 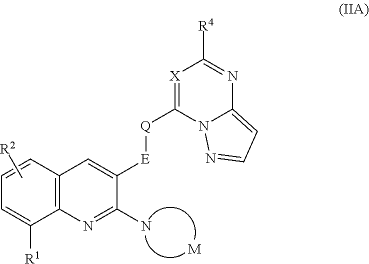
wherein E, Q, M, R¹, R² and R⁴ are as defined in claim 1.

3. A compound as claimed in claim 1 represented by formula (IIB) or an N-oxide thereof, or a pharmaceutically acceptable salt or solvate thereof: 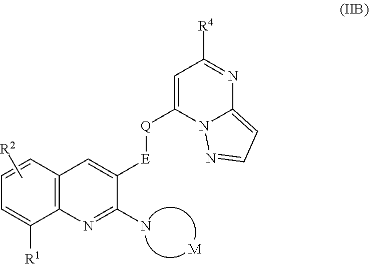
wherein E, Q, M, R¹, R² and R⁴ are as defined in claim 1.

4. A compound as claimed in claim 1 wherein M represents the residue of a monocyclic ring selected from pyrrolidin-1-yl, oxazolidin-3-yl, piperidin-1-yl, piperazin-1-yl and morpholin-4-yl, any of which rings may be optionally substituted by one or more substituents independently selected from halogen, C₁₋₆ alkyl, heteroaryl, C₁₋₆ alkoxy, difluoromethoxy, trifluoromethoxy, C₁₋₆ alkoxy(C₁₋₆)alkyl, C₁₋₆ alkylthio, C₁₋₆ alkylsulphonyl, hydroxy, hydroxy(C₁₋₆)alkyl, amino(C₁₋₆)alkyl, cyano, trifluoromethyl, oxo, C₂₋₆ alkylcarbonyl, hydroxy(C₁₋₆)alkylcarbonyl, di(C₁₋₆)alkylamino-(C₁₋₆)alkylcarbonyl, (C₃₋₇)cycloalkylcarbonyl, heteroarylcarbonyl, carboxy, carboxy-(C₁₋₆)alkyl, C₂₋₆ alkoxycarbonyl, C₂₋₆ alkoxycarbonyl(C₁₋₆)alkyl, amino, C₁₋₆ alkylamino, di(C₁₋₆)alkylamino, phenylamino, pyridinylamino, C₂₋₆ alkylcarbonylamino, hydroxy-(C₁₋₆)alkylcarbonylamino, (C₁₋₆)alkylcarbonylamino(C₁₋₆)alkyl, (C₃₋₇)cycloalkylcarbonyl-amino, C₂₋₆ alkoxycarbonylamino, aminocarbonyl, (C₁₋₆)alkylaminocarbonyl and di(C₁₋₆)alkylaminocarbonyl(C₁₋₆)alkyl.

5. A compound as claimed in claim 1 wherein the moiety of which M is the residue is selected from pyrrolidin-1-yl, oxopyrrolidin-1-yl, hydroxy-acetylaminopyrrolidin-1-yl, acetylaminomethylpyrrolidin-1-yl, cyclopropylcarbonyl-aminopyrrolidin-1-yl, oxooxazolidin-3-yl, carboxypiperidin-1-yl, aminocarbonyl-piperidin-1-yl, methylaminocarbonylpiperidin-1-yl, thiazolylpiperazin-1-yl, pyridinyl-piperazin-1-yl, pyrazinylpiperazin-1-yl, hydroxyethylpiperazin-1-yl, oxopiperazin-1-yl, acetylpiperazin-1-yl, propionylpiperazin-1-yl, tert-butylcarbonylpiperazin-1-yl, hydroxy-acetylpiperazin-1-yl, (dimethylamino)acetylpiperazin-1-yl, cyclopropylcarbonyl-piperazin-1-yl, thienylcarbonylpiperazin-1-yl, pyridinylcarbonylpiperazin-1-yl, carboxymethylpiperazin-1-yl, methoxycarbonylpiperazin-1-yl, ethoxycarbonylmethyl-piperazin-1-yl, aminocarbonylpiperazin-1-yl, methylaminocarbonylpiperazin-1-yl, dimethylaminocarbonylmethylpiperazin-1-yl, 5,6-dihydro-8H-[1,2,4]triazolo[4,3-a]-pyrazin-7-yl and 3-methyl-5,6-dihydro-8H-[1,2,4]triazolo[4,3-a]-pyrazin-7-yl.

6. A compound as claimed in claim 1 wherein E represents methylene or (methyl)methylene.

7. A compound as claimed in claim 1 wherein Q represents N—R⁸, in which R⁸ is as defined in claim 1.

8. A compound as claimed in claim 1 wherein R¹ represents hydrogen, halogen or C₁₋₆ alkyl.

9. A compound as claimed in claim 1 wherein R² represents hydrogen or halogen.

10. A compound as claimed in claim 1 wherein R⁴ represents hydrogen, —OR^{a}, —SR^{a}, —SOR^{a}, —SO₂R^{a} or —NR^{b}R^{c}, in which R^{a}, R^{b} and R^{c} are as defined in claim 1.

11. A compound that is
(S)-4-(8-Chloro-3-{1-[2-(methylthio)pyrazolo[1,5-a][1,3,5]triazin-4-ylamino]ethyl}-quinolin-2-yl)piperazin-2-one;
(S)-4-(8-Chloro-3-{1-[2-(methylsulfonyl)pyrazolo[1,5-a][1,3,5]triazin-4-ylamino]ethyl}-quinolin-2-yl)piperazin-2-one;
(S)-4-{8-Chloro-3-[1-(pyrazolo[1,5-a][1,3,5]triazin-4-ylamino)ethyl]quinolin-2-yl}piperazin-2-one;
(S)-1-[4-(8-Methyl-3-{1-[2-(methylthio)pyrazolo[1,5-a][1,3,5]triazin-4-ylamino]ethyl}-quinolin-2-yl)piperazin-1-yl]ethanone;
1-[4-(8-Methyl-3-{(1S)-1-[2-(methylsulfinyl)pyrazolo[1,5-a][1,3,5]triazin-4-ylamino]-ethyl}quinolin-2-yl)piperazin-1-yl]ethanone;
(S)-1-(4-{8-Methyl-3-[1-(pyrazolo[1,5-a][1,3,5]triazin-4-ylamino)ethyl]quinolin-2-yl}piperazin-1-yl)ethanone;
(S)-4-{8-Chloro-3-[1-(pyrazolo[1,5-a]pyrimidin-7-ylamino)ethyl]quinolin-2-yl}-piperazin-2-one;
(S)—N-{1-[8-Methyl-2-(pyrrolidin-1-yl)quinolin-3-yl]ethyl}pyrazolo[1,5-a][1,3,5]triazin-4-amine;
(S)-4-{3-[1-(2-Aminopyrazolo[1,5-a][1,3,5]triazin-4-ylamino)ethyl]-8-chloroquinolin-2-yl}piperazin-2-one;
(S)-4-(8-Chloro-3-{1-[2-(methylamino)pyrazolo[1,5-a][1,3,5]triazin-4-ylamino]ethyl}quinolin-2-yl)piperazin-2-one;
(S)-4-{7-Fluoro-8-methyl-3-[1-(pyrazolo[1,5-a][1,3,5]triazin-4-ylamino)ethyl]quinolin-2-yl}piperazin-2-one;
(S)-4-(7-Fluoro-8-methyl-3-{1-[2-(methylamino)pyrazolo[1,5-a][1,3,5]triazin-4-ylamino]ethyl}quinolin-2-yl)piperazin-2-one;
2-(4-{8-Chloro-3-[(1S)-1-(pyrazolo[1,5-a]pyrimidin-7-ylamino)ethyl]quinolin-2-yl}piperazin-1-yl)ethanol;
(4-{8-Chloro-3-[(1S)-1-(pyrazolo[1,5-a]pyrimidin-7-ylamino)ethyl]quinolin-2-yl}piperazin-1-yl)acetic acid;
Ethyl(4-{8-chloro-3-[(1S)-1-(pyrazolo[1,5-a]pyrimidin-7-ylamino)ethyl]quinolin-2-yl}piperazin-1-yl)acetate
N-{[(3R)-1-{8-Chloro-3-[(1S)-1-(pyrazolo[1,5-a]pyrimidin-7-ylamino)ethyl]quinolin-2-yl}pyrrolidin-3-yl]methyl}acetamide;
1-{7-Fluoro-8-methyl-3-[(1S)-1-(pyrazolo[1,5-a]pyrimidin-7-ylamino)ethyl]quinolin-2-yl}piperidine-4-carboxamide;
N-[(3S)-1-{7-Fluoro-8-methyl-3-[(1S)-1-(pyrazolo[1,5-a]pyrimidin-7-ylamino)ethyl]-quinolin-2-yl}pyrrolidin-3-yl]cyclopropanecarboxamide;
N-[(3R)-1-{8-Chloro-3-[(1S)-1-(pyrazolo[1,5-a]pyrimidin-7-ylamino)ethyl]quinolin-2-yl}pyrrolidin-3-yl]-2-hydroxyacetamide;
(4-{7-Fluoro-8-methyl-3-[(1S)-1-(pyrazolo[1,5-a]pyrimidin-7-ylamino)ethyl]quinolin-2-yl}piperazin-1-yl)acetic acid;
1-(4-{8-Chloro-3-[(1S)-1-(pyrazolo[1,5-a]pyrimidin-7-ylamino)ethyl]quinolin-2-yl}piperazin-1-yl)-2-hydroxyethanone;
1-(4-{8-Chloro-3-[(1S)-1-(pyrazolo[1,5-a]pyrimidin-7-ylamino)ethyl]quinolin-2-yl}piperazin-1-yl)-2-(dimethylamino)ethanone formic acid salt;
1-{7-Fluoro-8-methyl-3-[(1S)-1-(pyrazolo[1,5-a]pyrimidin-7-ylamino)ethyl]quinolin-2-yl}piperidine-4-carboxylic acid;
2-(Dimethylamino)-1-(4-{7-fluoro-8-methyl-3-[(1S)-1-(pyrazolo[1,5-a]pyrimidin-7-ylamino)ethyl]quinolin-2-yl}piperazin-1-yl)ethanone;
1-{7-Fluoro-8-methyl-3-[(1S)-1-(pyrazolo[1,5-a]pyrimidin-7-ylamino)ethyl]quinolin-2-yl}-N-methylpiperidine-4-carboxamide;
1-{8-Chloro-3-[(1S)-1-(pyrazolo[1,5-a]pyrimidin-7-ylamino)ethyl]quinolin-2-yl}-N-methylpiperidine-4-carboxamide;
(S)—N,N-Dimethyl-2-(4-{7-fluoro-8-methyl-3-[1-(pyrazolo[1,5-a]pyrimidin-7-ylamino)-ethyl]quinolin-2-yl}piperazin-1-yl)acetamide;
4-{8-Methyl-3-[(pyrazolo[1,5-a]pyrimidin-7-ylamino)methyl]quinolin-2-yl}piperazin-2-one;
(S)-2,2-Dimethyl-1-(4-{7-fluoro-8-methyl-3-[1-(pyrazolo[1,5-a]pyrimidin-7-ylamino)-ethyl]quinolin-2-yl}piperazin-1-yl)propan-1-one;
4-{8-Methyl-3-[(pyrazolo[1,5-a][1,3,5]triazin-4-ylamino)methyl]quinolin-2-yl}piperazin-2-one;
4-{3-[(2-Aminopyrazolo[1,5-a][1,3,5]triazin-4-ylamino)methyl]-8-methylquinolin-2-yl}piperazin-2-one;
(S)—N-(1-{7-Fluoro-8-methyl-2-[4-(pyridin-2-yl)piperazin-1-yl]quinolin-3-yl}ethyl)-pyrazolo[1,5-a]pyrimidin-7-amine;
(S)-(Cyclopropyl)(4-{7-fluoro-8-methyl-3-[1-(pyrazolo[1,5-a]pyrimidin-7-ylamino)-ethyl]quinolin-2-yl}piperazin-1-yl)methanone;
(S)—N-(1-{7-Fluoro-8-methyl-2-[4-(pyrazin-2-yl)piperazin-1-yl]quinolin-3-yl}ethyl)-pyrazolo[1,5-a]pyrimidin-7-amine;
(S)-1-(4-{7-Fluoro-8-methyl-3-[1-(pyrazolo[1,5-a]pyrimidin-7-ylamino)ethyl]quinolin-2-yl}piperazin-1-yl)propan-1-one;
(S)—N-(1-{7-Fluoro-8-methyl-2-[4-(thiazol-2-yl)piperazin-1-yl]quinolin-3-yl}ethyl)-pyrazolo[1,5-a]pyrimidin-7-amine;
(S)—N-(1-{7-Fluoro-8-methyl-2-[4-(pyridin-3-yl)piperazin-1-yl]quinolin-3-yl}ethyl)-pyrazolo[1,5-a]pyrimidin-7-amine;
S)-(4-{7-Fluoro-8-methyl-3-[1-(pyrazolo[1,5-a]pyrimidin-7-ylamino)ethyl]quinolin-2-yl}piperazin-1-yl)(thien-2-yl)methanone;
(S)-(4-{7-Fluoro-8-methyl-3-[1-(pyrazolo[1,5-a]pyrimidin-7-ylamino)ethyl]quinolin-2-yl}piperazin-1-yl)(pyridin-4-yl)methanone;
(S)-1-(4-{7-Fluoro-3-[1-(pyrazolo[1,5-a]pyrimidin-7-ylamino)ethyl]quinolin-2-yl}-piperazin-1-yl)ethanone;
(S)—N-{1-[7-Fluoro-8-methyl-2-(pyrrolidin-1-yl)quinolin-3-yl]ethyl}pyrazolo[1,5-a]-pyrimidin-7-amine;
(S)-1-(4-{7-Fluoro-8-methyl-3-[1-(pyrazolo[1,5-a]pyrimidin-7-ylamino)ethyl]quinolin-2-yl}piperazin-1-yl)ethanone;
(S)-4-{3-[1-(2-Aminopyrazolo[1,5-a][1,3,5]triazin-4-ylamino)ethyl]-7-fluoro-8-methyl-quinolin-2-yl}piperazin-2-one;
(S)-4-{7-Fluoro-8-methyl-3-[1-(pyrazolo[1,5-a]pyrimidin-7-ylamino)ethyl]quinolin-2-yl}piperazin-2-one;
(S)-4-{7-Fluoro-3-[1-(pyrazolo[1,5-a]pyrimidin-7-ylamino)ethyl]quinolin-2-yl}piperazin-2-one;
(S)-4-{5,7-Difluoro-3-[1-(pyrazolo[1,5-a]pyrimidin-7-ylamino)ethyl]quinolin-2-yl}-piperazin-2-one;
(S)-1-(4-{8-Chloro-3-[1-(pyrazolo[1,5-a]pyrimidin-7-ylamino)ethyl]quinolin-2-yl}-piperazin-1-yl)ethanone;
(S)-1-(4-{3-[1-(2-Aminopyrazolo[1,5-a][1,3,5]triazin-4-ylamino)ethyl]-8-chloroquinolin-2-yl}piperazin-1-yl)ethanone;
(S)-1-(4-{8-Chloro-3-[1-(2-methoxypyrazolo[1,5-a][1,3,5]triazin-4-ylamino)ethyl]-quinolin-2-yl}piperazin-1-yl)ethanone;
(S)-1-(4-{8-Chloro-3-[1-(pyrazolo[1,5-a][1,3,5]triazin-4-ylamino)ethyl]quinolin-2-yl}-piperazin-1-yl)ethanone;
(S)-1-(4-{5,7-Difluoro-3-[1-(pyrazolo[1,5-a]pyrimidin-7-ylamino)ethyl]quinolin-2-yl}-piperazin-1-yl)ethanone;
1-(4-{7-Fluoro-8-methyl-3-[(pyrazolo[1,5-a][1,3,5]triazin-4-ylamino)methyl]quinolin-2-yl}piperazin-1-yl)ethanone;
(S)-1-(4-{5,7-Difluoro-3-[1-(pyrazolo[1,5-a][1,3,5]triazin-4-ylamino)ethyl]quinolin-2-yl}piperazin-1-yl)ethanone;
1-(4-{7-Fluoro-8-methyl-3-[(pyrazolo[1,5-a]pyrimidin-7-ylamino)methyl]quinolin-2-yl}-piperazin-1-yl)ethanone;
(S)-1-(4-{3-[1-(Pyrazolo[1,5-a]pyrimidin-7-ylamino)ethyl]quinolin-2-yl}piperazin-1-yl)-ethanone;
Methyl 4-{7-fluoro-8-methyl-3-[(1S)-1-(pyrazolo[1,5-a]pyrimidin-7-ylamino)ethyl]-quinolin-2-yl}piperazine-1-carboxylate;
4-{7-Fluoro-8-methyl-3-[(1S)-1-(pyrazolo[1,5-a]pyrimidin-7-ylamino)ethyl]quinolin-2-yl}-N-methylpiperazine-1-carboxamide;
4-{7-Fluoro-8-methyl-3-[(1S)-1-(pyrazolo[1,5-a]pyrimidin-7-ylamino)ethyl]quinolin-2-yl}piperazine-1-carboxamide;
(R)-4-{8-Chloro-3-[1-(pyrazolo[1,5-a]pyrimidin-7-ylamino)ethyl]quinolin-2-yl}-piperazin-2-one;
1-{7-Fluoro-8-methyl-3-[(S)-1-(pyrazolo[1,5-a]pyrimidin-7-ylamino)ethyl]quinolin-2-yl}pyrrolidin-2-one;
N-{(S)-1-[2-(5,6-Dihydro-8H-[1,2,4]triazolo[4,3-a]pyrazin-7-yl)-7-fluoro-8-methyl-quinolin-3-yl]ethyl}pyrazolo[1,5-a]pyrimidin-7-ylamine;
N-{(S)-1-[7-Fluoro-8-methyl-2-(3-methyl-5,6-dihydro-8H-[1,2,4]triazolo[4,3-a]pyrazin-7-yl)quinolin-3-yl]ethyl}pyrazolo[1,5-a]pyrimidin-7-ylamine; or
3-{7-Fluoro-8-methyl-3-[(S)-1-(pyrazolo[1,5-a]pyrimidin-7-ylamino)ethyl]quinolin-2-yl}oxazolidin-2-one.

12. (canceled)

13. (canceled)

14. A pharmaceutical composition comprising a compound of formula (I) as defined in claim 1 or an N-oxide thereof, or a pharmaceutically acceptable salt or solvate thereof, in association with a pharmaceutically acceptable carrier.

15. (canceled)

16. A method for the treatment and/or prevention of a disorder for which the administration of a selective PI3K inhibitor is indicated which comprises administering to a patient in need of such treatment an effective amount of a compound of formula (I) as defined in claim 1 or an N-oxide thereof, or a pharmaceutically acceptable salt or solvate thereof.
